SEQUENCE LISTING (6 sequences)

SEQ ID NO: 1 - DNA, 59 bp, synthetic construct
  source 1..59: mol_type = other DNA
gttaactaattaatttaagaaggagatataaatatgagtaaaggagaagaacttttcac

SEQ ID NO: 2 - DNA, 41 bp, synthetic construct
  source 1..41: mol_type = other DNA
atttagaataggcgcgcccttacttgtacagctcgtccatg

SEQ ID NO: 3 - DNA, 43 bp, synthetic construct
  source 1..43: mol_type = other DNA
gcatttagaataggcgcgcccttacttgtacagctcgtccatg

SEQ ID NO: 4 - DNA, 49 bp, synthetic construct
  source 1..49: mol_type = other DNA
gttaactaattaatttaagaaggagatataaatatggtgagcaagggcg

SEQ ID NO: 5 - DNA, 20 bp, synthetic construct
  source 1..20: mol_type = other DNA
ggcgcgcctattctaaatgc

SEQ ID NO: 6 - DNA, 41 bp, synthetic construct
  source 1..41: mol_type = other DNA
atctccttcttaaattaattagttaacgaattcgatattac

(19) United States
(12) Patent Application Publication
(10) Pub. No.: US 2026/0284011 A1
(43) Pub. Date: Sep. 24, 2026

(54) COMPOUNDS FOR RESTORING SUSCEPTIBILITY TO BETA-LACTAM ANTIBIOTICS

(71) Applicant: UNIVERSITY OF NOTRE DAME DU LAC, South Bend, IN (US)

(72) Inventors: Shahriar MOBASHERY, South Bend, IN (US); Van T. NGUYEN, South Bend, IN (US); Jed F. FISHER, South Bend, IN (US); Mayland CHANG, South Bend, IN (US)

(73) Assignee: UNIVERSITY OF NOTRE DAME DU LAC, South Bend, IN (US)

(21) Appl. No.: 19/167,049

(22) PCT Filed: Mar. 19, 2024
(86) PCT No.: PCT/US2024/020594

§ 371 Date: Sep. 19, 2025

Related U.S. Application Data
(60) Provisional application No. 63/491,136, filed on Mar. 20, 2023.

(51) Int. Cl. A61K 31/4184; A61K 31/43; A61K 31/69; A61P 31/04; C07D 235/28; C07F 5/04

(52) U.S. Cl. CPC A61K 31/4184; A61K 31/43; A61K 31/69; A61P 31/04; C07D 235/28; C07F 5/04

(57) ABSTRACT

Infections by Staphylococcus aureus have been treated historically with b-lactam antibiotics. However, these antibiotics have become obsolete in methicillin-resistant S. aureus (MRSA) by acquisition of the bla and mec operons. The presence of the β-lactam antibiotic is detected by the sensor domains of the BlaR and/or MecR, and the information is transmitted to the cytoplasm, resulting in derepression of the antibiotic-resistance genes. We hypothesized that inhibition of the sensor domain would shut down this response system, and D-lactam susceptibility would be restored. An in-silico search of 11 million compounds led to a benzimidazole-based hit, and ultimately to the boronate 4. The X-ray structure of 4 is covalently engaged with the active-site serine of BlaR. Compound 4 potentiates by 16- to 4096-fold the activity of oxacillin and of meropenem against MRSA strains. The combination of 4 with oxacillin or meropenem shows efficacy in infected mice, validating the strategy.

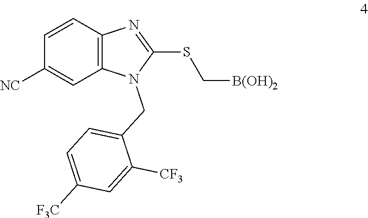 [SMILES: N#Cc1ccc2nc(SCB(O)O)n(Cc3ccc(C(F)(F)F)cc3C(F)(F)F)c2c1]

### RELATED APPLICATIONS

This application claims priority under 35 U.S.C. § 119(e) to U.S. Provisional Patent Application No. 63/491,136, filed Mar. 20, 2023, which is incorporated herein by reference.

### GOVERNMENT SUPPORT

This invention was made with government support under Grant No. 2R01 AI104987 awarded by the National Institutes of Health. The government has certain rights in the invention.

### REFERENCE TO SEQUENCE LISTING

The present application includes a Sequence Listing in electronic format as an xml file titled “501.092W01_SL” which was created on Mar. 11, 2024 and has a size of 6,481 bytes. The contents of xml file 501.092W01_SL are incorporated herein by reference.

### BACKGROUND OF THE INVENTION

The Gram-positive human pathogen Staphylococcus aureus is responsible for nosocomial (hospital-acquired) and community-acquired infections, including endocarditis, septicemia, pneumonia, skin and soft tissue infections and osteomyelitis. S. aureus strains expressing a class A (3-lactamase (BlaZ) gave resistance to the first-generation penicillins. Semi-synthetic structural refinements of these β-lactams culminated in 1959 in second-generation penicillins including methicillin to counter the effect of the BlaZ β-lactamase. These strains persist today and are referred to as methicillin-sensitive S. aureus (MSSA). The challenge of control of S. aureus infections by β-lactams increased with the isolation in 1961 of S. aureus strains resistant to methicillin and to other second-generation penicillins. A number of clonal origins for the variants of this pathogen has been identified, and they are collectively known as methicillin-resistant S. aureus (“MRSA”). MRSA continues as a global problem, accounting for a third of all nosocomial infections. MRSA causes roughly half of the deaths from bacterial infections in hospitals in the US. Resistance to β-lactams in both MSSA and MRSA strains is inducible: the organism does not activate the pathways to resistance until exposed to the antibiotic.

Accordingly, there is a need to interfere with this antibiotic-response pathway, and thus to render both MSSA and MRSA bacteria susceptible to β-lactams.

### SUMMARY

Infections by Staphylococcus aureus have been treated historically with β-lactam antibiotics. However, many of these antibiotics have become obsolete as chemotherapy for methicillin-resistant S. aureus (MRSA) as a result of the acquisition by this bacterium of the bla and mec operons. The presence of the β-lactam antibiotic is detected by the sensor domains of the BlaR and/or MecR proteins, and the information as to presence of the β-lactam antibiotic is transmitted to the cytoplasm, resulting in derepression of the antibiotic-resistance genes. We hypothesized that inhibition of the sensor domain would shut down this response system, and β-lactam susceptibility would be restored. Here, we outline a strategy to interfere with this antibiotic-response pathway, and thus to render both MSSA and MRSA bacteria susceptible to β-lactams.

Accordingly, this disclosure provides a compound of formula I:

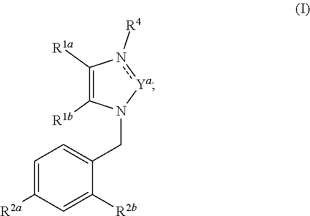

or a pharmaceutically acceptable salt thereof;
wherein,
   [symbol] is a double bond or a single bond;
   Y^{a} is

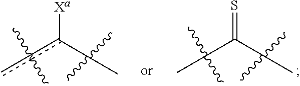

wherein X^{a} is SG³, H, halo, N(G¹)₂, SO₂G², or —CH₂R⁶;
G¹ is each independently H, —(C₁-C₆)alkyl, Boc, or Cbz;
G² is halo, OH, N(G¹)₂, —(C₁-C₆)alkyl, or —CH₂Ar;
G³ is —CH₂R⁷, H, —(C₁-C₆)alkyl, —(C₁-C₆)alkenyl, N(G¹)₂, or Ar;
G⁴ is OH, N(G¹)₂, or —O(C₁-C₆)alkyl;
Ar is a phenyl substituted with —O(C₁-C₆)alkyl;
R^{1a} and R^{1b} taken together with the carbon atoms to which they are attached in formula I form a benzo ring substituted with R^{1c}; or
   R^{1a} is H, CN, —C(═O)G⁴, —CH₂N(G¹)₂, or —(C₁-C₆)alkyl substituted with one or more OR²c; and
   R^{1b} is H, halo, or —(C₁-C₆)alkyl;
R^{1c} is CN, H, —C(═O)G⁴, —CH₂OR^{2c}, —CH₂N(G¹)₂, —CH₂N(G¹)COCH₃, or —CH₂N(G¹)CO(Y^{b})Ph, wherein Ph is substituted with R^{5a} and/or R^{5b}, and Y^{b} is —CH═CH— or —CH₂CH₂—;
R^{2a} and R^{2b} are each independently CF₃, H, halo, SF₅, or —(C₁-C₆)alkyl optionally substituted with one or more halo;
R^{2c} is each independently H or —(C₁-C₆)alkyl;
R⁴ is a lone pair when Y^{a} is

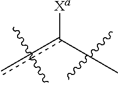

and [symbol] is a double bond; or
   R⁴ is H or —(C₁-C₆)alkyl when Y^{a} is

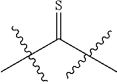 [SMILES: *C(*)=S]

and [symbol] is a single bond;
R^{5a} and R^{5b} are each independently H, halo, CF₃, SF₅, or —(C₁-C₆)alkyl optionally substituted with one or more halo;
R⁶ is halo, OR²c, —CH₂OR^{2c}, B(OR^{2c})₂, —CH₂B(OR^{2c})₂, B(pinanediol), —CH₂B(pinanediol), or

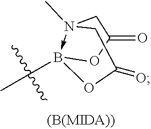 [SMILES: *[B-]12OC(=O)C[N+]1(C)CC(=O)O2]

and
   R⁷ is B(OR²c)₂, B(MIDA), Ar, or —(CH₂)ₙCN wherein n is 1 to 6.

This disclosure also provides a method for treating a bacterial infection, comprising administering a therapeutically effective amount of a compound described above to a subject infected with bacteria, wherein the bacterial infection is thereby treated.

Additionally, this disclosure provides a method for treating a methicillin-resistant Staphylococcus aureus (MRSA) infection, comprising administering a therapeutically effective amount of an antibiotic and a compound described above to a subject infected with MRSA, wherein the bacterial infection is thereby treated.

The invention provides novel compounds of Formulas I, IB, II, IIB, III, IIIB, IIIC, IV, IVB, or V, intermediates for the synthesis of compounds of Formulas I, IB, II, IIB, III, IIIB, IIIC, IV, IVB, or V, as well as methods of preparing compounds of Formulas I, IB, II, IIB, III, IIIB, IIIC, IV, IVB, or V. The invention also provides compounds of Formulas I, IB, II, IIB, III, IIIB, IIIC, IV, IVB, or V that are useful as intermediates for the synthesis of other useful compounds. The invention provides for the use of compounds of Formulas I, IB, II, IIB, III, IIIB, IIIC, IV, IVB, or V for the manufacture of medicaments useful for the treatment of bacterial infections in a mammal, such as a human.

The invention provides for the use of the compositions described herein for use in medical therapy. The medical therapy can be used to treat a bacterial infection. The invention also provides for the use of a composition as described herein for the manufacture of a medicament to treat an infection in a mammal, for example, an MRSA infection in a human. The medicament can include a pharmaceutically acceptable diluent, excipient, or carrier.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Schematic for the activation of BlaR. β-Lactam (top left, the β-lactam shown is oxacillin) recognition at the surface sensor domain leads to activation of the BlaR zinc-protease domain at the inner leaflet of the cytoplasmic membrane. This protease activity degrades BlaI. As a result of the loss of BlaI, the antibiotic-resistance genes of its operon, including that for BlaR itself, are derepressed. This structural model for the BlaR protein is based on that proposed by Belluzo et al (Sci. Rep. 9, 19558 (2019)). The mec operon is homologous to the bla system depicted in this figure, except that the gene mecA (for PBP2a) replaces the blaZ gene, mecI replaces blaI, and mecR replaces blaR.

FIG. 2A-C. (a) The modified bla operons depicted to the right replace the blaZ gene for the BlaZ β-lactamase with genes for fluorescent proteins. BlaR sensing of a β-lactam effects derepression of the bla operon as a result of BlaR-catalyzed proteolytic degradation of the BlaI repressor bound to P_{blaR} and P_{balZ} (FIG. 1). As a consequence, the fluorescent proteins are produced. Crystal structures of compound 2 (b) and of compound 4 (c) bound to the sensor domain of BlaR. Ser-389 hydrogen bonds to the hydroxy group of 2. In contrast Ser-389 is covalently bound to the boronate of 4.

FIG. 3A-F. Compound 4 was evaluated in (a) the checkerboard assay, (b) the time-kill assay, OXA at 32 mg·L⁻¹, compound 4 at 8 mg·L⁻¹, OXA:4 combinations at 16:4, 16:8, 32:4, and 8:8 mg·L⁻¹, and (c) neutropenic thigh infection with and without OXA using strain NRS21 (a, b, c, respectively), and with and without MEM using strain FPR3757 (USA300) (d, e, f, respectively). MEM at 2 mg·L⁻¹, compound 4 at 8 mg·L⁻¹, and combinations of MEM:4 at 1:4, 1:8, 2:4, and 2:8 mg·L⁻¹. OXA, MEM, or compound 4 did not show antibacterial activity at 24 h, when given as single-drug treatment. However, the combination of OXA or MEM and compound 4 showed 1.3- to 1.8-log reduction in bacterial counts. Neutropenic mice (n=5 mice/group) were given three subcutaneous doses of compound 4 (20 mg·kg⁻¹) or OXA (40 mg·kg⁻¹) at 1, 9, and 17 h after infection, or six intraperitoneal doses of MEM (50 mg·kg⁻¹) at 1, 5, 9, 13, 17, and 21 h after infection or a combination of OXA or MEM and 4 (OXA:4 or MEM:4). The infected thighs were collected at 24 h after infection and the homogenate was plated, followed by determination of bacterial counts (mean±standard error of the mean). * p<0.05 and *** p<0.001 compared to vehicle, as determined by Tukey's multiple comparison test.

FIG. 4. Comparison of the fluorescence intensity produced by S. aureus NRS144 strains harboring pCN52RI, pCN52RI-venus and pCN52RI-mCherry. Each fluorescent protein was induced by the β-lactam antibiotic CBAP. The ratios of fluorescence in the presence of CBAP to that in the absence of it were 3, 10 and 5 for GFP, Venus, and mCherry, respectively. Data were derived from four independent assays.

FIG. 5A-P. Checkerboard graphs of combination of compound 4 with antibiotics. (a) MEM in HFH29258, (b) MEM in HFH30495, (c) MEM in HI049, (d) MEM in NRS404, (e) MEM in NRS715, (f) OXA in NRS21, (g) OXA in NRS4, (h) OXA in NRS70, (i) OXA in NRS382, (j) OXA in NRS22, (k) FOX in FPR3757, (l) FOX in HFH30945, (m) FOX in H1049, (n) FOX in NRS715, (o) FOX in NRS404, (p) CPT in NRS404.

FIG. 6A-J. Time-kill assays of compound 4 with OXA, MEM, and CPT in various MRSA strains. (a) OXA in NRS21, (b) OXA in NRS70, (c) OXA in NRS382, (d) MEM in NRS21, (e) MEM in NRS70, (f) MEM in NRS404, (g) MEM in HFH29568, (h) MEM in HFH30495, (i) CPT in NRS21, and (j) CPT in NRS404. Neither OXA, MEM, CPT, or compound 4 by itself showed reduction in bacterial growth at 24 h. The combinations of OXA:4, MEM:4, or CPT:4 showed up to 3-log bacterial growth reduction.

FIG. 7. Concentrations of compound 4 in plasma and thighs of mice. Uninfected mice (n=3 per time point) received a single 25 mg·kg⁻¹ subcutaneous dose of compound 4. Terminal blood and thighs were collected, processed, and analyzed by UPLC with UV detection. Concentrations are expressed in μg/g tissue for thighs and in mg·mL⁻¹ in plasma.

FIG. 8A-D. Efficacy of compound 4 in the mouse neutropenic thigh infection model. Mice (n=5 per group) were made neutropenic (200 mg·kg⁻¹ intraperitoneal cyclophosphamide on days −4 and −1). Both thighs were infected intramuscularly with 100 μL of 10⁶ CFU·mL⁻¹. Mice were given OXA or compound 4 subcutaneously at 1, 9, and 17 h after infection, or MEM intraperitoneally at 1, 5, 9, 13, 17, and 21 h after infection, or a combination of OXA:4 or MEM:4. The thighs were excised at 24 h after infection, plated, and counted. Data are presented as mean±standard error of the mean, * p<0.05, ** p<0.01 by Tukey's multiple comparison test. (a) OXA at 20 mg·kg⁻¹ and compound 4 at 20 mg·kg⁻¹ in NRS21, (b) OXA at 30 mg·kg⁻¹ and compound 4 at 15 mg·kg⁻¹ in NRS21, (c) MEM at 50 mg·kg⁻¹ and compound 4 at 30 mg·kg⁻¹ in FPR3757, and (d) MEM at 100 mg/kg and compound 4 at 20 mg·kg⁻¹.

### DETAILED DESCRIPTION

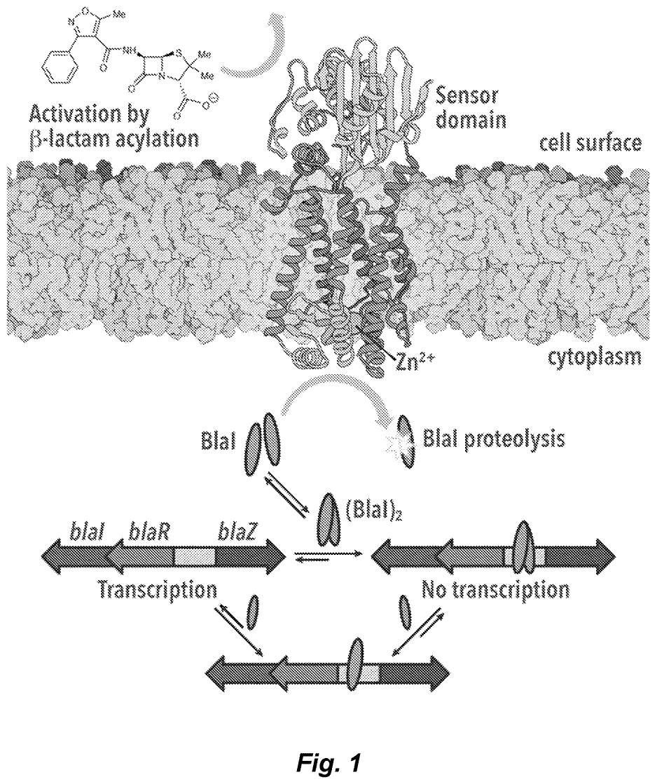

The resistance responses to β-lactam antibiotics in MSSA and MRSA are governed by the cognate bla and/or mec operons, respectively. Although the two operons are highly homologous, the bla system is better studied and is the more prevalent (FIG. 1). Regulation of production of β-lactamase and also of PBP2a—the two resistance determinants—is often due to the bla system, as the mec regulatory genes frequently contain deletions/truncations and mutations that inactivate them. Consequently, the bla system is mechanistically important. BlaR is the β-lactam sensor/signal transducer protein for which the full-length structure was recently revealed using cryo-EM (Nature 613, 375-382 (2023)). A key lysine in the β-lactam-binding site of BlaR is N^{ζ}-carboxylated as a modification that is indispensable for signal transduction. Upon encounter of the BlaR sensor domain with a β-lactam, general-base activation by the N^{ζ}-carboxylated lysine of the active-site serine transfers the acyl moiety of the β-lactam to the serine. This transfer is followed by irreversible NL decarboxylation of the lysine. We previously termed this reaction sequence as the “lysine NL decarboxylation switch” (J. Biol. Chem. 286, 31466-72 (2011)). Its consequence is the irreversible covalent capture by BlaR of the β-lactam, resulting in a conformational change across the entire BlaR protein as a mechanism for signal transduction through the membrane. This change activates the metalloprotease of the cytoplasmic domain of BlaR resulting in an autoproteolytic cleavage. This cytoplasmic metalloprotease domain then recognizes and degrades the gene repressor BlaI (or MecI) protein, with attendant derepression of the resistance genes, blaZ (for a class A β-lactamase) and/or mecA (for penicillin-binding protein 2a; PBP2a). The breadth of the S. aureus β-lactamase activity is limited to penicillins. However, PBP2a is especially problematic, as it confers resistance to virtually all commercial β-lactam antibiotics. The respective repressor proteins (BlaI or MecI) bind as monomers and/or dimers to the promoter regions of their operons. This binding prevents transcription of the genes (FIG. 1). Thus, the functions of the bla and mec operons hinge on acylation of the BlaR (or the cognate MecR) protein by the β-lactam. In the absence of the β-lactam the resistance response is silent. Accordingly, if one could inhibit acylation of the sensor domain of BlaR protein by the (3-lactam antibiotic, the ability of MSSA or MRSA to manifest resistance to the β-lactam class of antibiotics would be abrogated.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

The recitation of a), b), c), . . . or i), ii), iii), or the like in a list of components or steps do not confer any particular order unless explicitly stated.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, the patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The compound and compositions described herein may be administered with additional compositions to prolong stability and activity of the compositions, or in combination with other therapeutic drugs.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms.

The disclosure illustratively described herein may be suitably practiced in the absence of any element or elements, limitation or limitations which is not specifically disclosed herein. This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); for heterocyclic synthesis see Hermanson, Greg T., Bioconjugate Techniques, Third Edition, Academic Press, 2013.

The formulas and compounds described herein can be modified using protecting groups. Suitable amino and carboxy protecting groups are known to those skilled in the art (see for example, Protecting Groups in Organic Synthesis, Second Edition, Greene, T. W., and Wuts, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and referenced cited therein.

The term “halo” or “halide” refers to fluoro, chloro, bromo, or iodo. Similarly, the term “halogen” refers to fluorine, chlorine, bromine, and iodine.

The term “alkyl” refers to a branched or unbranched hydrocarbon having, for example, from 1-20 carbon atoms, and often 1-12, 1-10, 1-8, 1-6, or 1-4 carbon atoms; or for example, a range between 1-20 carbon atoms, such as 2-6, 3-6, 2-8, or 3-8 carbon atoms. As used herein, the term “alkyl” also encompasses a “cycloalkyl”, defined below. Examples include, but are not limited to, methyl, ethyl, 1-propyl, 2-propyl (iso-propyl), 1-butyl, 2-methyl-1-propyl (isobutyl), 2-butyl (sec-butyl), 2-methyl-2-propyl (t-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, and the like. The alkyl can be unsubstituted or substituted, for example, with a substituent described below or otherwise described herein. The alkyl can also be optionally partially or fully unsaturated. As such, the recitation of an alkyl group can include an alkenyl group or an alkynyl group. The alkyl can be a monovalent hydrocarbon radical, as described and exemplified above, or it can be a divalent hydrocarbon radical (i.e., an alkylene).

An alkylene is an alkyl group having two free valences at a carbon atom or two different carbon atoms of a carbon chain. Similarly, alkenylene and alkynylene are respectively an alkene and an alkyne having two free valences at two different carbon atoms, or an alkenylene can have the two free valences on the same carbon.

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantyl, and the like. The cycloalkyl can be unsubstituted or substituted. The cycloalkyl group can be monovalent or divalent and can be optionally substituted as described for alkyl groups. The cycloalkyl group can optionally include one or more cites of unsaturation, for example, the cycloalkyl group can include one or more carbon-carbon double bonds, such as, for example, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and the like.

The term “heteroatom” refers to any atom in the periodic table that is not carbon or hydrogen. Typically, a heteroatom is O, S, N, P. The heteroatom may also be a halogen, metal or metalloid.

The term “heterocycloalkyl” or “heterocyclyl” refers to a saturated or partially saturated monocyclic, bicyclic, or polycyclic ring containing at least one heteroatom selected from nitrogen, sulfur, oxygen, preferably from 1 to 3 heteroatoms in at least one ring. Each ring is preferably from 3- to 10-membered, more preferably 4 to 7 membered. Examples of suitable heterocycloalkyl substituents include pyrrolidinyl, tetrahydrofuranyl, tetrahydrothiofuranyl, piperidinyl, piperazinyl, tetrahydropyranyl, morpholinyl, 1,3-diazapanyl, 1,4-diazapanyl, 1,4-oxazepanyl, and 1,4-oxathiapanyl. The group may be a terminal group or a bridging group.

The term “aryl” refers to an aromatic hydrocarbon group derived from the removal of at least one hydrogen atom from a single carbon atom of a parent aromatic ring system. The radical attachment site can be at a saturated or unsaturated carbon atom of the parent ring system. The aryl group can have from 6 to 30 carbon atoms, for example, about 6-10 carbon atoms. The aryl group can have a single ring (e.g., phenyl) or multiple condensed (fused) rings, wherein at least one ring is aromatic (e.g., naphthyl, dihydrophenanthrenyl, fluorenyl, or anthryl). Typical aryl groups include, but are not limited to, radicals derived from benzene, naphthalene, anthracene, biphenyl, and the like. The aryl can be unsubstituted or optionally substituted with a substituent described below. For example, a phenyl moiety or group may be substituted with one or more substituents R^{X} where R^{X} is at the ortho-, meta-, or para-position, and X is an integer variable of 1 to 5.

The term “heteroaryl” refers to a monocyclic, bicyclic, or tricyclic ring system containing one, two, or three aromatic rings and containing at least one nitrogen, oxygen, or sulfur atom in an aromatic ring. The heteroaryl can be unsubstituted or substituted, for example, with one or more, and in particular one to three, substituents, as described in the definition of “substituted”. Typical heteroaryl groups contain 2-20 carbon atoms in the ring skeleton in addition to the one or more heteroatoms, wherein the ring skeleton comprises a 5-membered ring, a 6-membered ring, two 5-membered rings, two 6-membered rings, or a 5-membered ring fused to a 6-membered ring. Examples of heteroaryl groups include, but are not limited to, 2H-pyrrolyl, 3H-indolyl, 4H-quinolizinyl, acridinyl, benzo[b]thienyl, benzothiazolyl, β-carbolinyl, carbazolyl, chromenyl, cinnolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrrolyl, quinazolinyl, quinolyl, quinoxalinyl, thiadiazolyl, thianthrenyl, thiazolyl, thienyl, triazolyl, tetrazolyl, and xanthenyl. In one embodiment the term “heteroaryl” denotes a monocyclic aromatic ring containing five or six ring atoms containing carbon and 1, 2, 3, or 4 heteroatoms independently selected from non-peroxide oxygen, sulfur, and N(Z) wherein Z is absent or is H, O, alkyl, aryl, or (C₁-C₆)alkylaryl. In some embodiments, heteroaryl denotes an ortho-fused bicyclic heterocycle of about eight to ten ring atoms derived therefrom, particularly a benzo-derivative or one derived by fusing a propylene, trimethylene, or tetramethylene diradical thereto.

As used herein, the term “substituted” or “substituent” is intended to indicate that one or more (for example, in various embodiments, 1-10; in other embodiments, 1-6; in some embodiments 1, 2, 3, 4, or 5; in certain embodiments, 1, 2, or 3; and in other embodiments, 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound. Suitable indicated groups include, e.g., alkyl, alkenyl, alkynyl, alkoxy, haloalkyl, hydroxyalkyl, aryl, heteroaryl, heterocyclyl, cycloalkyl, alkanoyl, alkoxycarbonyl, amino, alkylamino, dialkylamino, carboxyalkyl, alkylthio, alkylsulfinyl, and alkylsulfonyl. Substituents of the indicated groups can be those recited in a specific list of substituents described herein, or as one of skill in the art would recognize, can be one or more substituents selected from alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, and cyano. Suitable substituents of indicated groups can be bonded to a substituted carbon atom include F, Cl, Br, I, OR′, OC(O)N(R′)₂, CN, CF₃, OCF₃, R′, O, S, C(O), S(O), methylenedioxy, ethylenedioxy, N(R′)₂, SR′, SOR′, SO₂R, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂NHC(O)R′, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety (e.g., (C₁-C₆)alkyl), and wherein the carbon-based moiety can itself be further substituted. When a substituent is monovalent, such as, for example, F or Cl, it is bonded to the atom it is substituting by a single bond. When a substituent is divalent, such as O, it is bonded to the atom it is substituting by a double bond; for example, a carbon atom substituted with O forms a carbonyl group, C═O.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof, such as racemic mixtures, which form part of the present invention. Many organic compounds exist in optically active forms, i.e., they have the ability to rotate the plane of plane-polarized light. In describing an optically active compound, the prefixes D and L, or R and S. are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate (defined below), which may occur where there has been no stereoselection or stereospecificity in a chemical reaction or process.

The term “IC₅₀” is generally defined as the concentration required to inhibit a specific biological or biochemical function by half, or to kill 50% of the cells in a designated time period, typically 24 hours.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof, such as racemic mixtures, which form part of the present invention. Many organic compounds exist in optically active forms, i.e., they have the ability to rotate the plane of plane-polarized light. In describing an optically active compound, the prefixes D and L, or R and S. are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate (defined below), which may occur where there has been no stereoselection or stereospecificity in a chemical reaction or process.

The terms “racemic mixture” and “racemate” refer to an equimolar mixture of two enantiomeric species, devoid of optical activity.

The term “organoboronic acid” or “boronic acid” means a compound represented by R*—B(OH), where R* is an organic group that is bonded to the boron through a boron-carbon bond.

The moiety B(pinanediol) is

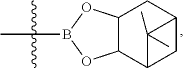 [SMILES: *B1OC2CC3CC(C2O1)C3(C)C]

which moiety can be either the (+)- or the (−)-moiety.

The term “tautomer” refers to a structural isomer of a chemical compound that readily interconverts resulting from the relocation of a hydrogen atom within the compound. For example, the 2-thio derivatives of imidazole and benzimidazole described herein (i.e., compounds 8, 9, 14, 15, 17, 22, 23, 25, 27, 29) have a tautomeric equilibrium, where in some embodiments, the thioxo tautomer dominates:

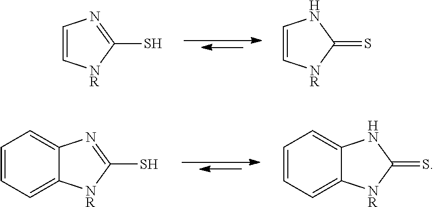

The abbreviation “Boc” refers to the amine protecting group tert-butyloxycarbonyl.

The abbreviation “Cbz” refers to the amine protecting group benzyloxycarbonyl.

The abbreviation “B(MIDA)” refers to a N-methylimidodiacetic boronic acid ester group.

### STATEMENTS OF THE TECHNOLOGY

1. A compound of formula I, IB, II, or IIB:

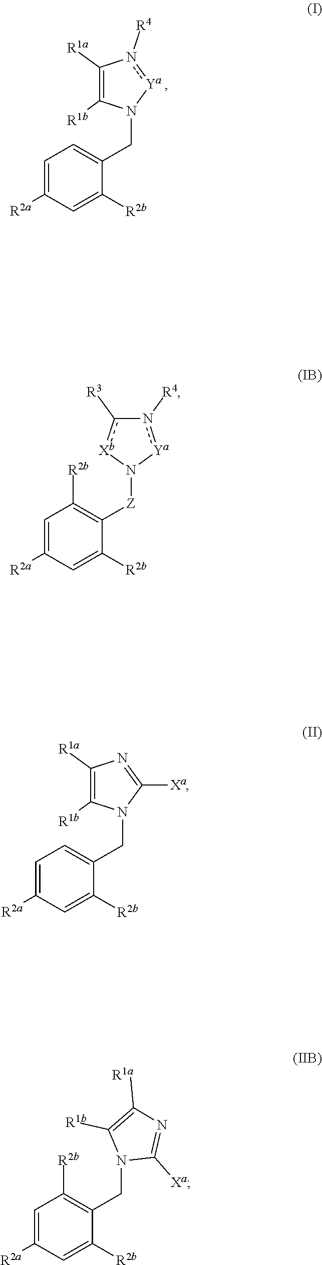

or a pharmaceutically acceptable salt and/or tautomers thereof;
wherein,
   [symbol] is each independently a single bond or a double bond, optionally, wherein at least one [symbol] is a double bond;
   X^{a} is SG³, H, halo, N(G¹)₂, SO₂G², or —CH₂R⁶;
   X^{b} is CR^{2c}, NH, or N;
   Y^{a} is

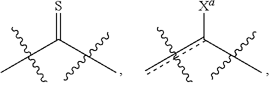

or N;
Z is —CH₂— or a single bond attached to the aromatic carbon atom of the phenyl group of formula IIB and the nitrogen atom of the heterocycle of formula IIB;
G¹ is each independently H, —(C₁-C₆)alkyl, Boc, or Cbz;
G² is halo, OH, N(G¹)₂, —(C₁-C₆)alkyl, or —CH₂Ar;
G³ is —CH₂R⁷, H, —(C₁-C₆)alkyl, —(C₁-C₆)alkenyl, N(G¹)₂, or Ar;
G⁴ is OH, N(G¹)₂, or —O(C₁-C₆)alkyl;
Ar is a phenyl substituted with —O(C₁-C₆)alkyl;
R^{1a} and R^{1b} taken together with the carbon atoms to which they are attached in formulas I, II and IIB to form a benzo ring substituted with R^{1c}; or
   R^{1a} is H, CN, —C(═O)G⁴, —CH₂N(G¹)₂, or —(C₁-C₆)alkyl substituted with one or more OR^{2c}, and R^{1b} is H, halo, —(C₁-C₆)alkyl, or R^{1c};
R^{1c} is CN, H, —C(═O)G⁴, —CH₂OR^{2c}, —CH₂N(G¹)₂, —CH₂N(G¹)COCH₃, or —CH₂N(G¹)CO(Y^{b})Ph, wherein Ph is substituted with R^{5a} and/or R^{5b}, and Y^{b} is —CH═CH— or —CH₂CH₂—;
R^{2a} and R^{2b} are each independently CF₃, H, halo, or —(C₁-C₆)alkyl optionally substituted with one or more halo;
R^{2c} is each independently H or —(C₁-C₆)alkyl;
R³ is —CH₂OR²c, B(OR²c)₂, or

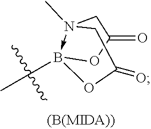 [SMILES: *[B-]12OC(=O)C[N+]1(C)CC(=O)O2]

R⁴ is a lone pair, H, or —(C₁-C₆)alkyl;
R^{5a} and R^{5b} are each independently H, halo, CF₃, or —(C₁-C₆)alkyl optionally substituted with one or more halo; or
   R^{2a}, R^{2b}, R⁴, R^{5a}, and R^{5b} are each independently H, Cl, F, Br, I, SF₅, OCF₃, or CF₃;
R⁶ is halo, OR^{2c}, —CH₂OR²c, B(OR²c)₂, —CH₂B(OR²c)₂, B(pinanediol), —CH₂B(pinanediol), or B(MIDA); and
R⁷ is B(MIDA), Ar, B(OR^{2c})₂, or —(CH₂)ₙCN wherein n is 1 to 6.
2. The compound of statement 1, wherein X^{a} is H, Cl, NH₂, SO₃H, SO₂F, SO₂Cl, SO₂NH₂, SO₂Et, SO₂CH₂Ar, SH, SNH₂, SEt, S(allyl), SAr, SCH₂R⁷, SCH₂Ar, or CH₂R⁶, wherein Ar is an anisole moiety.
3. The compound of statement 1 or 2, wherein Ar is para-anisole.
4. The compound of any one of statements 1-3, wherein X^{b} is CH, NH, or N.
5. The compound of any one of statements 1-4, wherein R^{1a} is H, CN, CO₂H, CONH₂, CO₂Et, CH₂NH₂, CH₂NHBoc, CH₂NHCbz, CH₂OH, CH(OH)CH(OH)CH₂OH, or CH(OH)CH(OH)CH(OH)CH₂OH.
6. The compound of any one of statements 1-5, wherein R^{1a} is

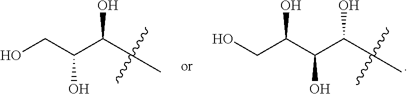 [SMILES: *[C@H](O)[C@H](O)CO ; *[C@@H](O)[C@@H](O)[C@H](O)CO]

7. The compound of any one of statements 1-6, wherein R^{1c} is H, CN, CO₂H, CO₂Me, CH₂OH, CH₂NH₂, CH₂NHCOCH₃, CH₂NHCOCH₂CH₂Ph, or CH₂NHCOCH═CHPh, wherein Ph is substituted with R^{5a} and R^{5b}.
8. The compound of any one of statements 1-7, wherein R^{1c} is —CH₂NHCOCH₂CH₂Ph or —CH₂NHCOCH═CHPh wherein the double bond is trans, wherein Ph is

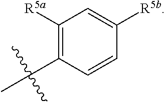

9. The compound of any one of statements 1-8, wherein R^{2a}, R^{2b}, R⁴, R^{5a}, and R^{5b} are each independently H, Cl, F, Br, I, SF₅, OCF₃, or CF₃.
10. The compound of any one of statements 1-9, wherein R³ is CH₂OH, B(OH)₂, or

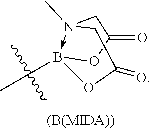 [SMILES: *[B-]12OC(=O)C[N+]1(C)CC(=O)O2]

11. The compound of any one of statements 1-10, wherein R⁷ is CN, CH₂CN, CH₂CH₂CN, B(OH)₂, or B(MIDA).
12. The compound of any one of statements 1-11, wherein R⁶ is Br, OH, CH₂OH, B(OH)₂, CH₂B(OH)₂, B((+)pinanediol), CH₂B((+)pinanediol), or B(MIDA).
13. The compound of any one of statements 1-12, as represented by formula III, IIIB, IIIC, or IV:

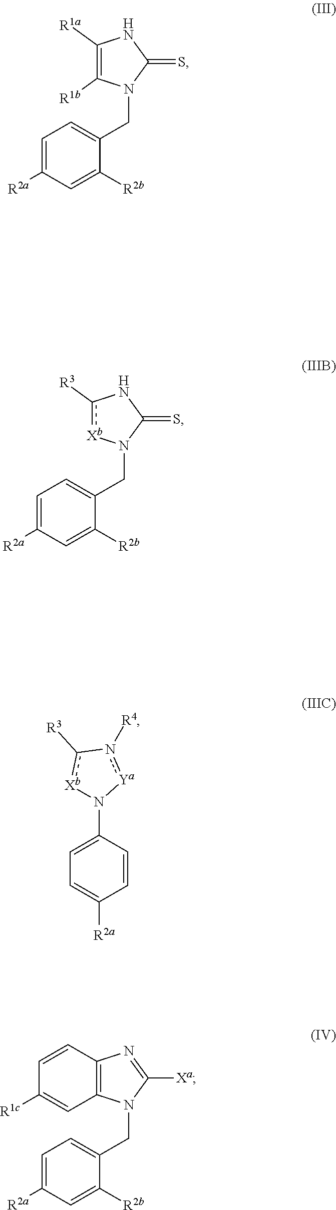

or a pharmaceutically acceptable salt and/or tautomers thereof.
14. The compound of any one of statements 1-12, as represented by formula IVB or V:

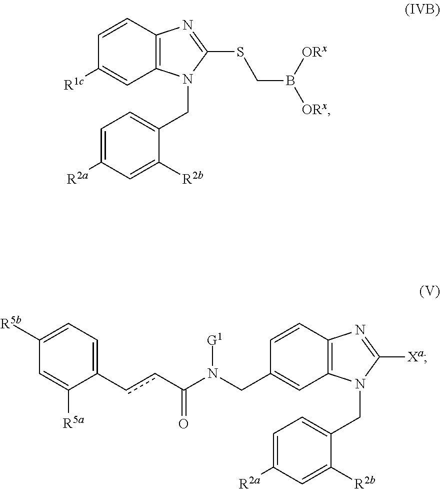

or a pharmaceutically acceptable salt and/or tautomers thereof,
   wherein for formula IVB, R^{x} is H or —(C₁-C₆)alkyl; or both R^{x} taken together with the boron atom to which they are attached via the oxygen atom form B(MIDA) or B(pinanediol).
15. The compound of statement 1, wherein the compound is compound 4, 59, 32, or 49:

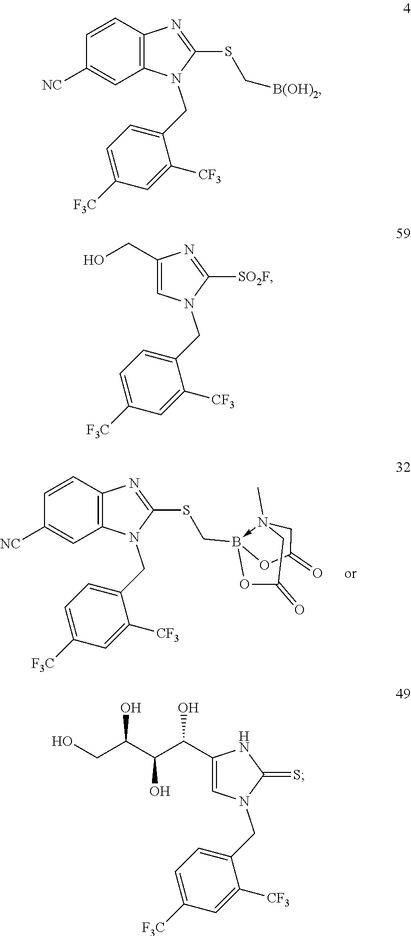 [SMILES: N#Cc1ccc2nc(SCB(O)O)n(Cc3ccc(C(F)(F)F)cc3C(F)(F)F)c2c1 ; O=S(=O)(F)c1nc(CO)cn1Cc1ccc(C(F)(F)F)cc1C(F)(F)F ; C[N+]12CC(=O)O[B-]1(CSc1nc3ccc(C#N)cc3n1Cc1ccc(C(F)(F)F)cc1C(F)(F)F)OC(=O)C2 ; OC[C@@H](O)[C@H](O)[C@H](O)c1cn(Cc2ccc(C(F)(F)F)cc2C(F)(F)F)c(=S)[nH]1]

or a pharmaceutically acceptable salt and/or tautomers thereof.
16. The compound of statement 1, wherein the compound is:

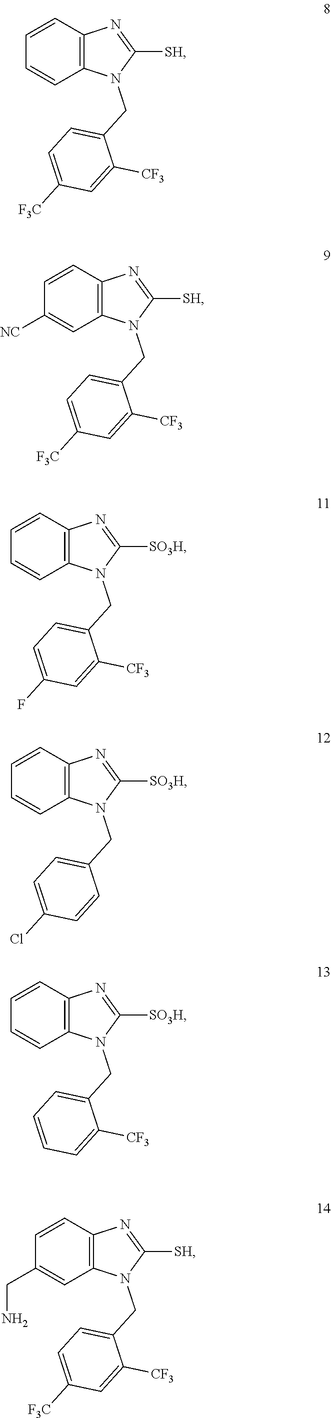 [SMILES: FC(F)(F)c1ccc(Cn2c(S)nc3ccccc32)c(C(F)(F)F)c1 ; N#Cc1ccc2nc(S)n(Cc3ccc(C(F)(F)F)cc3C(F)(F)F)c2c1 ; O=S(=O)(O)c1nc2ccccc2n1Cc1ccc(F)cc1C(F)(F)F ; O=S(=O)(O)c1nc2ccccc2n1Cc1ccc(Cl)cc1 ; O=S(=O)(O)c1nc2ccccc2n1Cc1ccccc1C(F)(F)F ; NCc1ccc2nc(S)n(Cc3ccc(C(F)(F)F)cc3C(F)(F)F)c2c1]

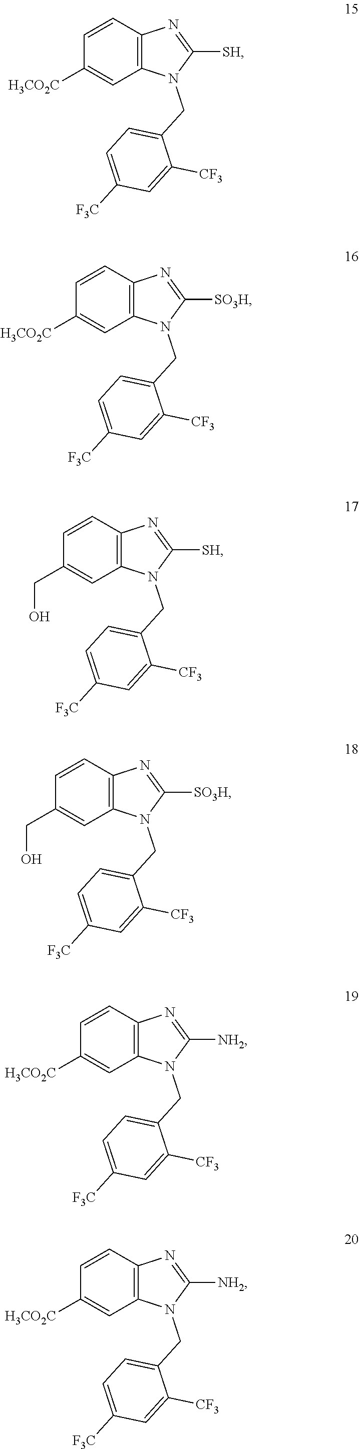 [SMILES: COC(=O)c1ccc2nc(S)n(Cc3ccc(C(F)(F)F)cc3C(F)(F)F)c2c1 ; COC(=O)c1ccc2nc(S(=O)(=O)O)n(Cc3ccc(C(F)(F)F)cc3C(F)(F)F)c2c1 ; OCc1ccc2nc(S)n(Cc3ccc(C(F)(F)F)cc3C(F)(F)F)c2c1 ; O=S(=O)(O)c1nc2ccc(CO)cc2n1Cc1ccc(C(F)(F)F)cc1C(F)(F)F ; COC(=O)c1ccc2nc(N)n(Cc3ccc(C(F)(F)F)cc3C(F)(F)F)c2c1 ; COC(=O)c1ccc2nc(N)n(Cc3ccc(C(F)(F)F)cc3C(F)(F)F)c2c1]

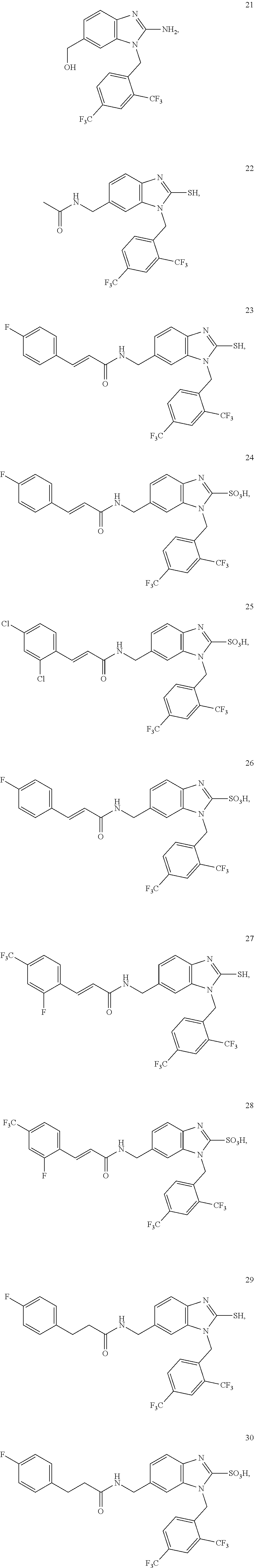

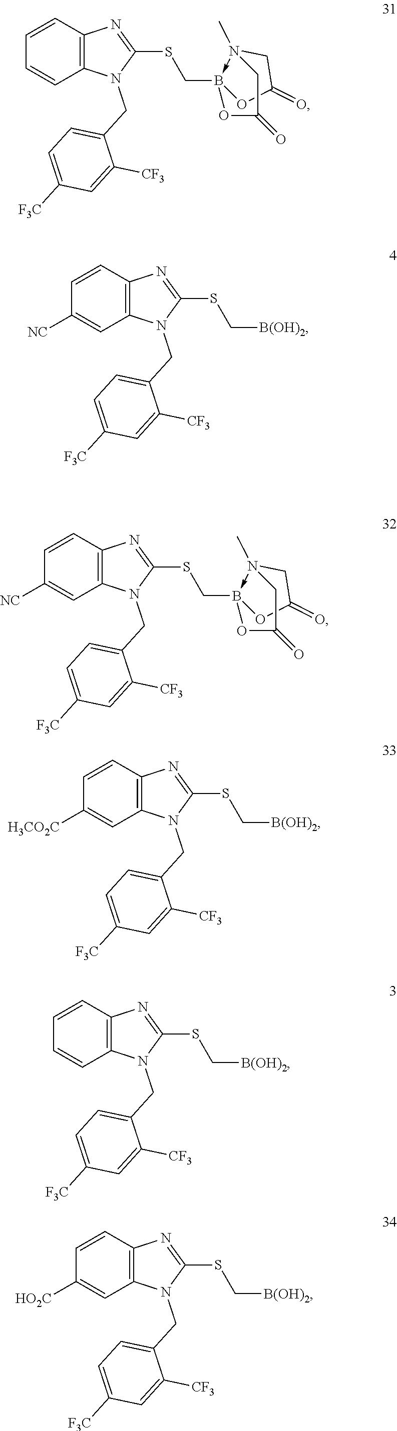 [SMILES: C[N+]12CC(=O)O[B-]1(CSc1nc3ccccc3n1Cc1ccc(C(F)(F)F)cc1C(F)(F)F)OC(=O)C2 ; N#Cc1ccc2nc(SCB(O)O)n(Cc3ccc(C(F)(F)F)cc3C(F)(F)F)c2c1 ; C[N+]12CC(=O)O[B-]1(CSc1nc3ccc(C#N)cc3n1Cc1ccc(C(F)(F)F)cc1C(F)(F)F)OC(=O)C2 ; COC(=O)c1ccc2nc(SCB(O)O)n(Cc3ccc(C(F)(F)F)cc3C(F)(F)F)c2c1 ; OB(O)CSc1nc2ccccc2n1Cc1ccc(C(F)(F)F)cc1C(F)(F)F ; O=C(O)c1ccc2nc(SCB(O)O)n(Cc3ccc(C(F)(F)F)cc3C(F)(F)F)c2c1]

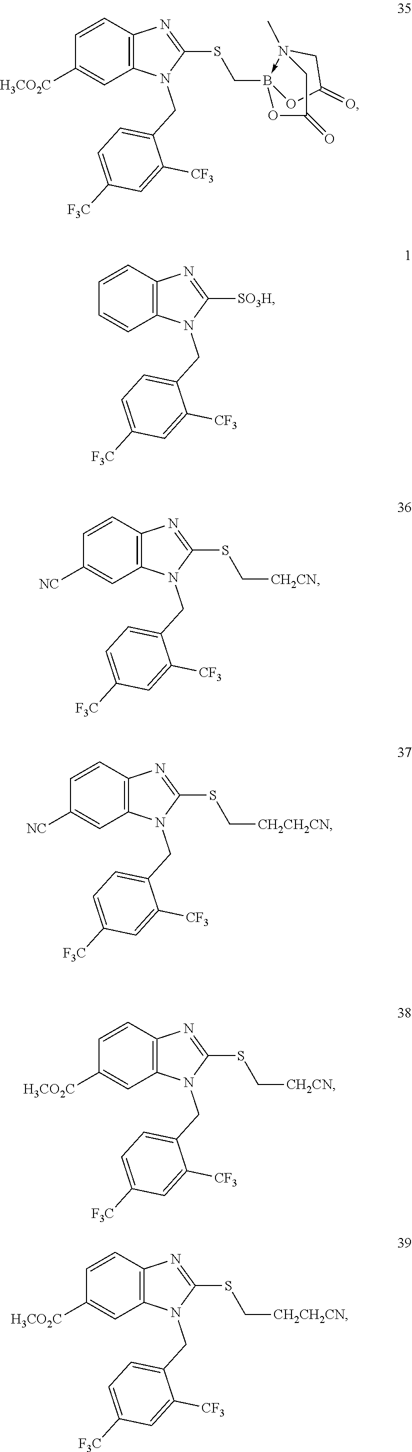

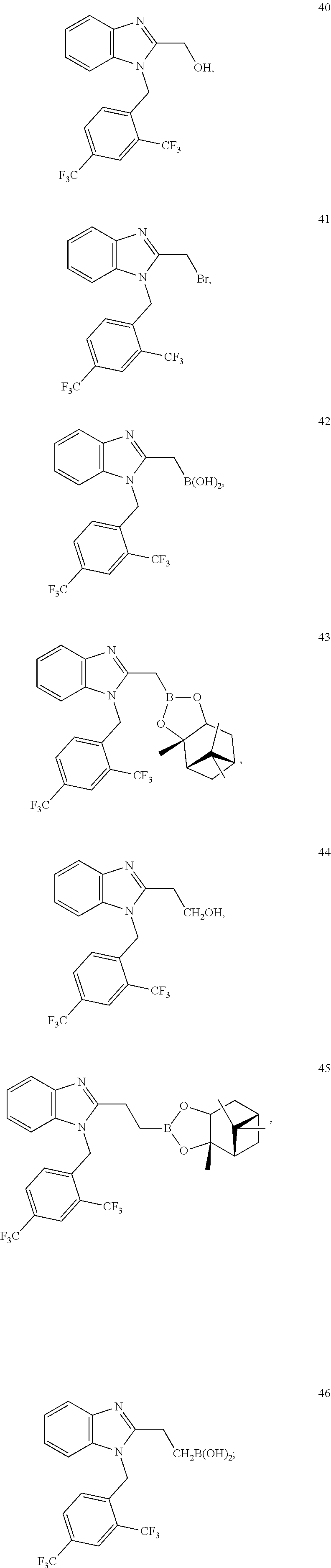

or a pharmaceutically acceptable salt and/or tautomers thereof.
17. The compound of statement 1, wherein the compound is:

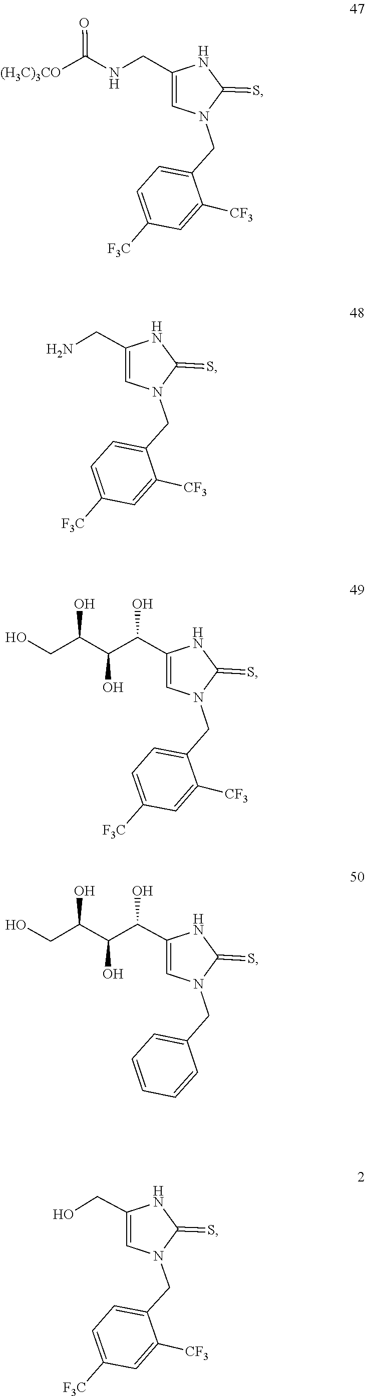

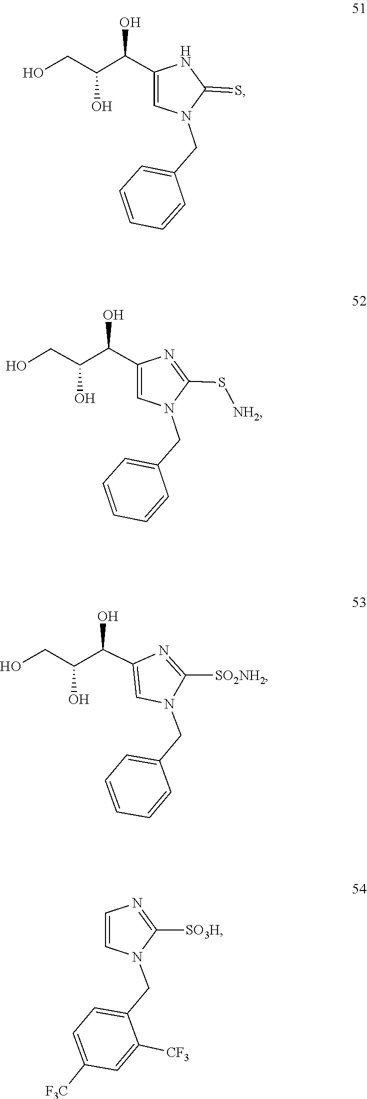 [SMILES: OC[C@@H](O)[C@@H](O)c1cn(Cc2ccccc2)c(=S)[nH]1 ; NSc1nc([C@H](O)[C@H](O)CO)cn1Cc1ccccc1 ; NS(=O)(=O)c1nc([C@H](O)[C@H](O)CO)cn1Cc1ccccc1 ; O=S(=O)(O)c1nccn1Cc1ccc(C(F)(F)F)cc1C(F)(F)F]

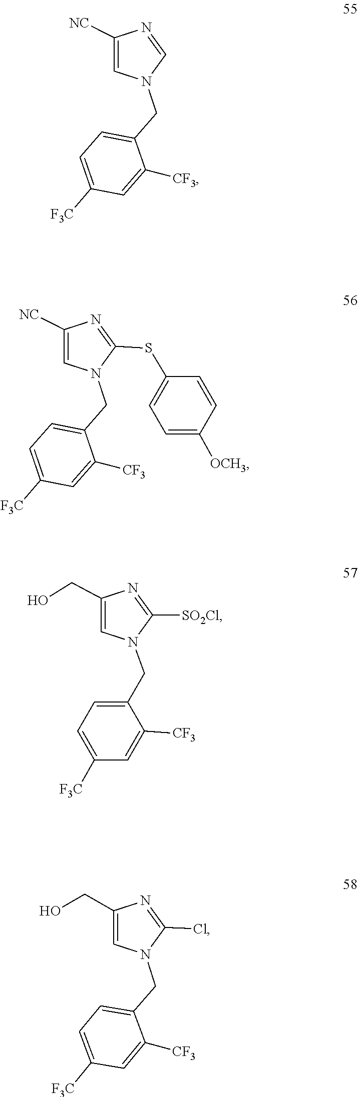 [SMILES: N#Cc1cn(Cc2ccc(C(F)(F)F)cc2C(F)(F)F)cn1 ; COc1ccc(Sc2nc(C#N)cn2Cc2ccc(C(F)(F)F)cc2C(F)(F)F)cc1 ; O=S(=O)(Cl)c1nc(CO)cn1Cc1ccc(C(F)(F)F)cc1C(F)(F)F ; OCc1cn(Cc2ccc(C(F)(F)F)cc2C(F)(F)F)c(Cl)n1]

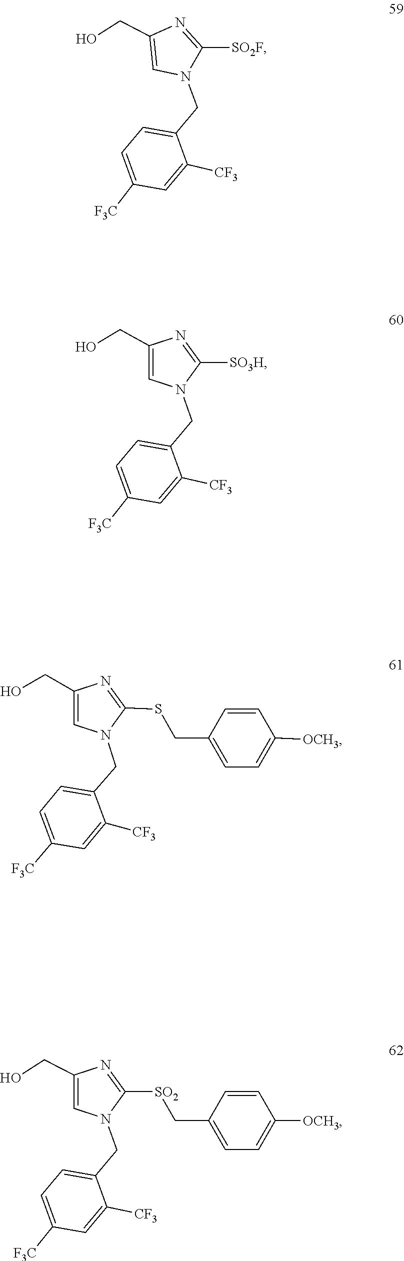 [SMILES: O=S(=O)(F)c1nc(CO)cn1Cc1ccc(C(F)(F)F)cc1C(F)(F)F ; O=S(=O)(O)c1nc(CO)cn1Cc1ccc(C(F)(F)F)cc1C(F)(F)F ; COc1ccc(CSc2nc(CO)cn2Cc2ccc(C(F)(F)F)cc2C(F)(F)F)cc1 ; COc1ccc(CS(=O)(=O)c2nc(CO)cn2Cc2ccc(C(F)(F)F)cc2C(F)(F)F)cc1]

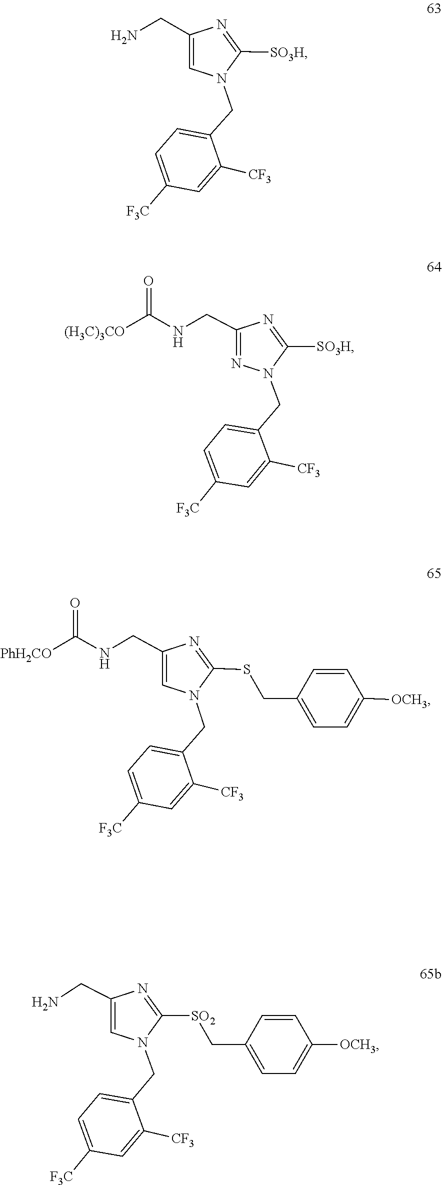

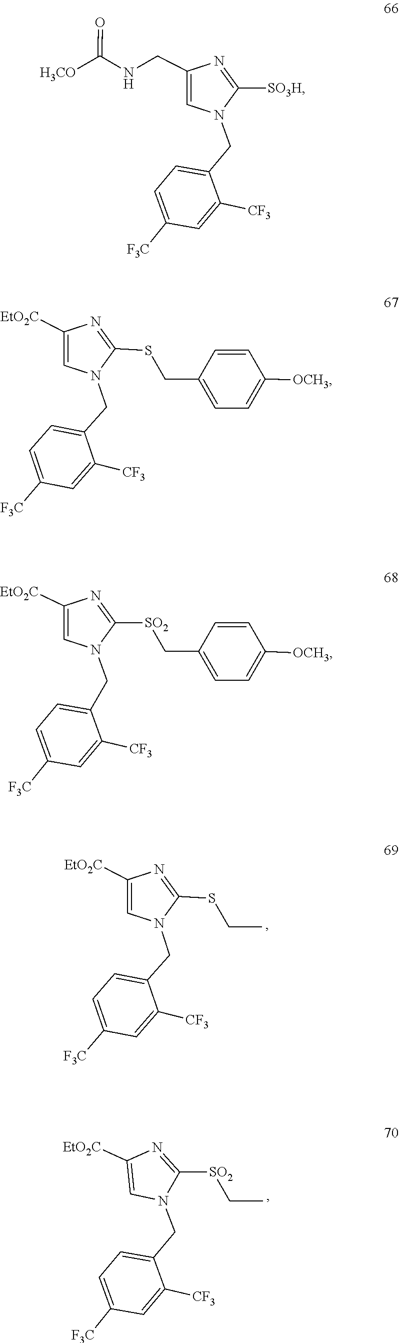 [SMILES: COC(=O)NCc1cn(Cc2ccc(C(F)(F)F)cc2C(F)(F)F)c(S(=O)(=O)O)n1 ; CCOC(=O)c1cn(Cc2ccc(C(F)(F)F)cc2C(F)(F)F)c(SCc2ccc(OC)cc2)n1 ; CCOC(=O)c1cn(Cc2ccc(C(F)(F)F)cc2C(F)(F)F)c(S(=O)(=O)Cc2ccc(OC)cc2)n1 ; CCOC(=O)c1cn(Cc2ccc(C(F)(F)F)cc2C(F)(F)F)c(SCC)n1 ; CCOC(=O)c1cn(Cc2ccc(C(F)(F)F)cc2C(F)(F)F)c(S(=O)(=O)CC)n1]

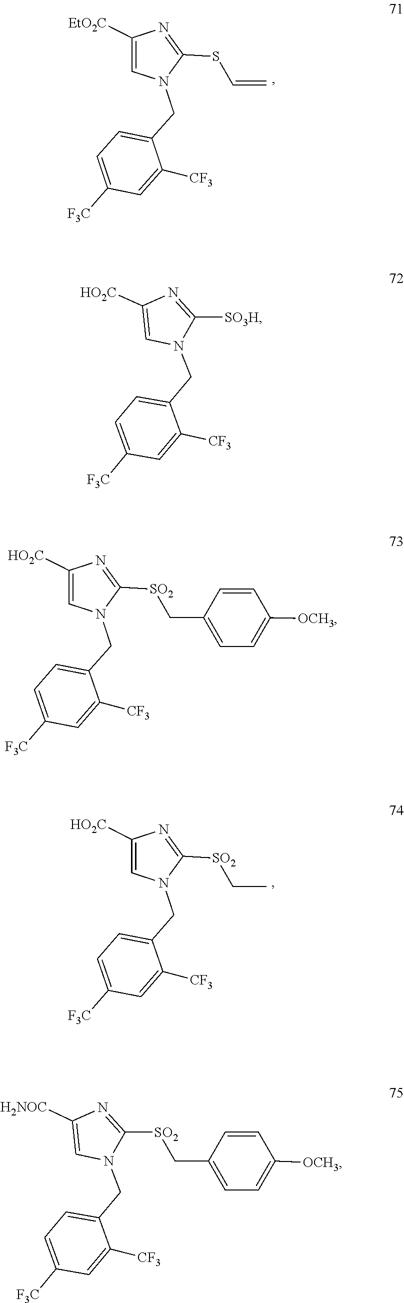 [SMILES: C=CSc1nc(C(=O)OCC)cn1Cc1ccc(C(F)(F)F)cc1C(F)(F)F ; O=C(O)c1cn(Cc2ccc(C(F)(F)F)cc2C(F)(F)F)c(S(=O)(=O)O)n1 ; COc1ccc(CS(=O)(=O)c2nc(C(=O)O)cn2Cc2ccc(C(F)(F)F)cc2C(F)(F)F)cc1 ; CCS(=O)(=O)c1nc(C(=O)O)cn1Cc1ccc(C(F)(F)F)cc1C(F)(F)F ; COc1ccc(CS(=O)(=O)c2nc(C(N)=O)cn2Cc2ccc(C(F)(F)F)cc2C(F)(F)F)cc1]

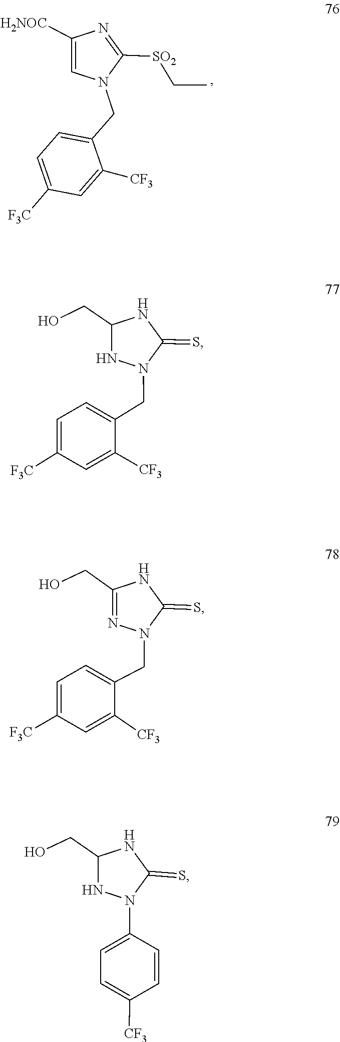 [SMILES: CCS(=O)(=O)c1nc(C(N)=O)cn1Cc1ccc(C(F)(F)F)cc1C(F)(F)F ; OCC1NC(=S)N(Cc2ccc(C(F)(F)F)cc2C(F)(F)F)N1 ; OCc1nn(Cc2ccc(C(F)(F)F)cc2C(F)(F)F)c(=S)[nH]1 ; OCC1NC(=S)N(c2ccc(C(F)(F)F)cc2)N1]

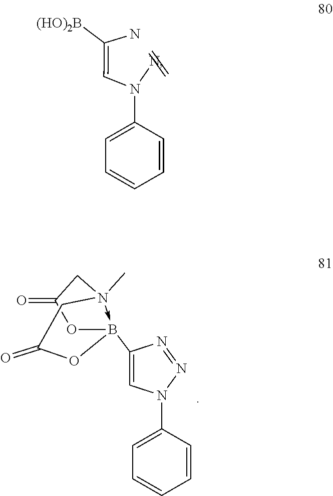

or a pharmaceutically acceptable salt and/or tautomers thereof.
18. A pharmaceutical composition comprising the compound of any one of statements 1-17, and a pharmaceutically acceptable diluent or carrier.
19. The pharmaceutical composition of statement 18, further comprising an antibiotic, wherein the antibiotic is optionally a β-lactam antibiotic.
20. A method for treating a bacterial infection, comprising administering a therapeutically effective amount of a compound of any one of statements 1-17 to a subject infected with bacteria, wherein the bacterial infection is thereby treated.
21. The method of statement 20, wherein the bacteria is Staphylococcus aureus, methicillin-sensitive Staphylococcus aureus (MSSA), or methicillin-resistant Staphylococcus aureus (MRSA).
22. A method for restoring the susceptibility of a beta-lactam antibiotic-resistant bacteria to a bacteria susceptible to a beta-lactam antibiotic comprising administering to a patient infected with a beta-lactam antibiotic-resistant bacteria an effective amount of a compound of any one of statements 1-17, wherein administration of the compound restores the susceptibility of the bacteria to a beta-lactam antibiotic.
23. The method of statement 22 comprising treating the beta-lactam antibiotic-resistant bacteria by further administering to the patient an effective amount of a beta-lactam antibiotic.
24. A method for treating a methicillin-resistant Staphylococcus aureus (MRSA) infection, comprising administering a therapeutically effective amount of an antibiotic and a compound of any one of statements 1-17 to a subject infected with MRSA, wherein the bacterial infection is thereby treated.
25. The method of statement 23 or 24, wherein the antibiotic is a penicillin, a cephalosporin, a carbapenem, or a monobactam.
26. The method of any one of statements 23-25, wherein the antibiotic is oxacillin, methicillin, flucloxacillin, cefazolin, cefoxitin, meropenem, or aztreonam.
27. The method of any one of statements 18-26, wherein the compound is (((1-(2,4-bis(trifluoro-methyl)benzyl)-6-cyano-1H-benzo[d]imidazol-2-yl)thio)methyl)boronic acid (compound 4).

### Results.

Virtual Screening for Inhibitors. The availability of X-ray structures for the sensor domain of BlaR enabled the virtual screening of 11 million compounds from the ZINC database. This library was evaluated using the Glide program. Successively more accurate screening advanced the top 10% scorers to the standard precision (SP) mode. The top one percent advanced from the SP mode to the extra-precision (XP) mode. The top one percent ranked molecules were rescored separately by the Goldscore, Chemscore, and ASP functions of Gold, which score based of distinct principles. The top one percent of compounds from each one of the four scoring functions were pooled together. After removing redundant molecules, these top-scoring compounds were further prioritized on the basis of their predicted fit within the binding site, the dynamic simulation of the complex, their availability for purchase, and their ease of synthesis. A collection of 100 compounds was assembled, by purchase and by synthesis, and tested for inhibition of BlaR.

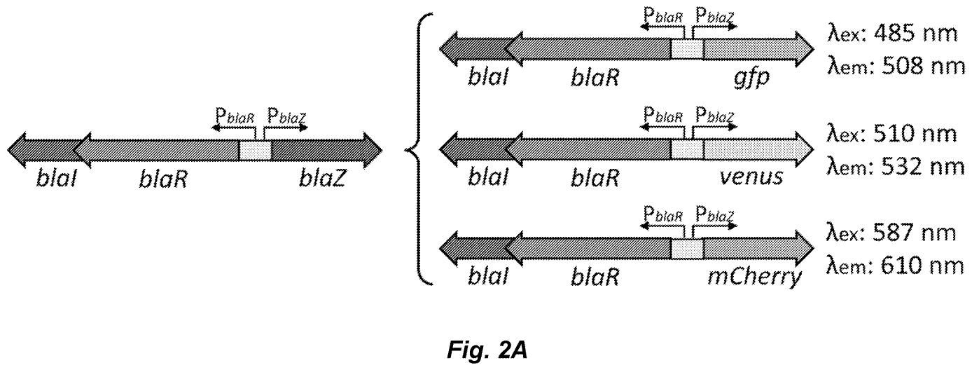

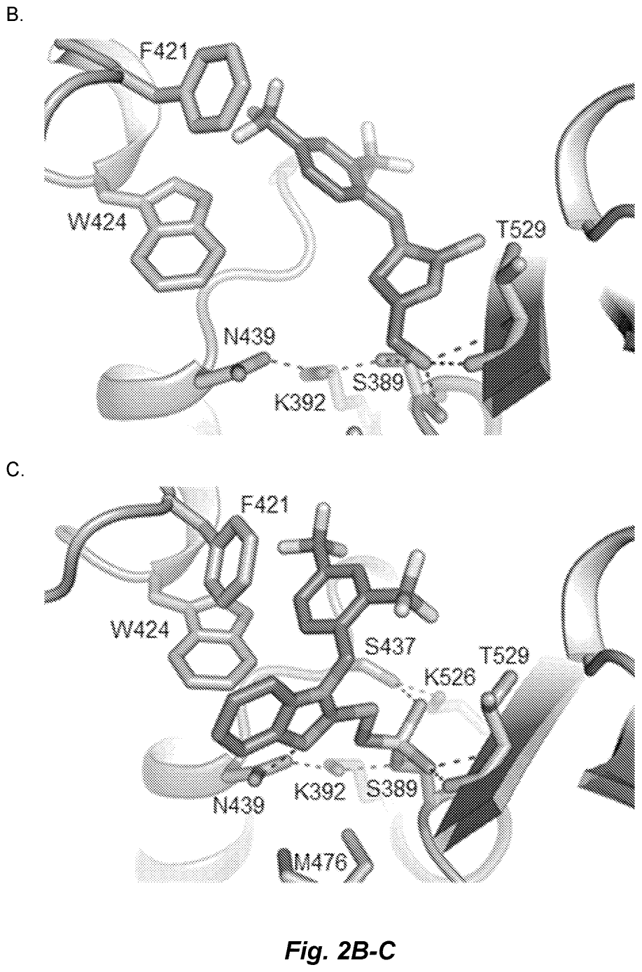

Live-Cell Fluorescent Assay in Discovery and Mechanism of Action. We conceived of fluorescent constructs in the bla operon, for the purpose of screening (FIG. 2a). We removed the gene for the β-lactamase (blaZ) and put in its place individually the genes for the Green Fluorescent Protein (GFP), Venus (a yellow-fluorescent protein) or mCherry (a red-fluorescent protein) in the template plasmid pCN52. The three plasmids were named pCN52RI, pCN52RI-Venus and pCN52RI-mCherry, respectively. The genes for the fluorescent proteins were transcribed under the promoter of the blaZ gene and were regulated by the proteins BlaR and BlaI, as is the case for the wild-type bla operon.

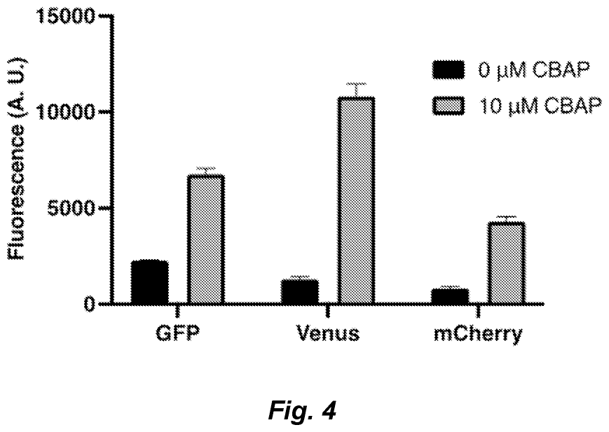

FIG. 2a shows the constructs and the excitation and emission wavelengths for each. We transformed separately S. aureus NRS144—a laboratory strain that accepts a foreign gene and harbors neither the bla nor the mec operons—with each of these plasmids. In the absence of induction of the system by β-lactam antibiotics, synthesis of the fluorescent protein does not occur. When induced, the fluorescent protein is produced and is detected (akin to expression of the resistance genes blaZ or mecA in typical MSSA and MRSA). We used 10 mg·L⁻¹ of the strong inducer penicillin 2-(2-carboxyphenyl)-benzoyl-6-aminopenicillanic acid (CBAP, minimal-inhibitory concentration (MIC) of 64 mg·L⁻¹ against the NRS144 strain). The transformed strains were grown in tryptic soy broth medium for overnight culture and strain maintenance and incubated in cation-adjusted Mueller-Hinton broth medium for inhibitor screening in a 96-well microplate format. The culture without CBAP addition was the negative control. In order to select a desirable reporter construct, we compared the intensity of the fluorescence produced from each construct at the wavelengths given in FIG. 2a using the Cytation-5 multi-mode microplate reader. Among the three constructs, pCN52RI-Venus produced the best fluorescence intensity and pCN52RI-mCherry was the second best (FIG. 4).

The collection of the 100 compounds from the in-silico search was screened against S. aureus NRS144_{pCN52RI-Venus}. The screening strategy was as follows. Bacteria grown to OD₆₀₀ of 0.25 were exposed concurrently to CBAP (10 mg·L⁻¹) and the test compound (100 mg·L⁻¹). Fluorescence was recorded after two hours of incubation. OD₆₀₀ values were also recorded to identify compounds with inherent antibacterial activity. Compound 1 (Chart 1) emerged—82% diminished fluorescence as an indication of the extent of BlaR inhibition—as a suitable template for structure elaboration. This effort led to the synthesis of several dozen benzimidazole and imidazole compounds. The progression of the iterative design is depicted in Chart 1 for four compounds, culminating in 4. The boronate inhibitors showed the best inhibitory activities. The X-ray structures for the complexes of compounds 2 (non-covalent) and 4 (covalent) with the BlaR sensor domain are shown respectively in FIG. 2b (2.0 Å resolution) and FIG. 2c (1.97 Å resolution). Compound 4 makes a covalent bond with the catalytic Ser389 and creates a network of hydrogen bonds with the BlaR active site (FIG. 2c).

Chart 1. Evolution of the structural template of the benzimidazole and imidazole classes of BlaR inhibitors into β-lactam potentiators. The percentages of BlaR and of bacterial growth inhibition at 100 mg·L⁻¹ are given below the structures; N=2 replicates. Data shows inhibition of a) BlaR induction and b) Bacterial growth.

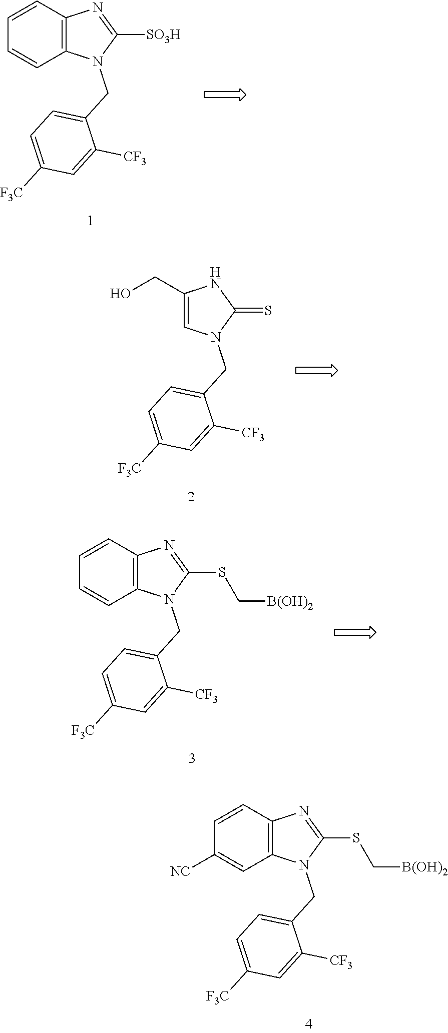 [SMILES: O=S(=O)(O)c1nc2ccccc2n1Cc1ccc(C(F)(F)F)cc1C(F)(F)F ; OCc1cn(Cc2ccc(C(F)(F)F)cc2C(F)(F)F)c(=S)[nH]1 ; OB(O)CSc1nc2ccccc2n1Cc1ccc(C(F)(F)F)cc1C(F)(F)F ; N#Cc1ccc2nc(SCB(O)O)n(Cc3ccc(C(F)(F)F)cc3C(F)(F)F)c2c1]

$$a ) 82 + 0.7 \%$$

$$92 \pm 1.4 \%$$

$$94 \pm 2.1 \%$$

$$99 \pm 0 \%$$

$$b ) none$$

$$none$$

$$11 \pm 1.4 \%$$

$$24 \pm 2.1 \%$$

Potentiation of Activities of/36-Lactam Antibiotics. Compound 4 was evaluated as an adjuvant for β-lactam antibiotics, per design principles. We assayed for potentiation of the activity of oxacillin (OXA), meropenem (MEM), cefoxitin (FOX), and ceftaroline (CPT; currently used clinically) against 40 MSSA and MRSA strains (Table 1 and Table 2 for comprehensive data). Potentiated activities of OXA and MEM against 20 selected MSSA and MRSA strains are presented in Table 1.

TABLE 1
MICs of OXA, OXA with 8 mg · L⁻¹ of 4, MEM, and MEM with 8 mg ·
L⁻¹ of 4 against selected 20 MRSA and MSSA strains.

| | | | | | MIC (mg · L⁻¹) | | | | | | |
|---|---|---|---|---|---|---|---|---|---|---|---|
| | | | | | | | 4 + | | | 4 + | |
| Entry | Strain name | designation | SCCmec^{a} | bla^{b} | 4 | OXA | OXA^{c} | | MEM | MEM^{c} | |
| 1 | NCTC8325 | MSSA | — | − | 16 | 0.25 | 0.25 | | 0.13 | 0.06 | (2) |
| 2 | COL (NRS100) | MRSA | I | − | 32 | 512 | 256 | (2) | 16 | 1 | (16) |
| 3^{e} | JE2 | MRSA | IV | − | 16 | 128 | 64 | (2) | 4 | 0.06 | (64) |
| 4 | ATCC29213 | MSSA | — | + | 16 | 2 | 0.5 | (4) | 0.13 | 0.06 | (2) |
| 5 | LIM2 (NRS36) | MRSA | I | + | 16 | 64 | 1 | (64) | 8 | 0.016 | (512) |
| 6 | HIP5836 | MRSA/ | II | + | 16 | 128 | 0.25 | (512) | 8 | 0.004 | (2048) |
| | (NRS4) | VISA | | | | | | | | | |
| 7 | HIP07930 | MRSA/ | II | + | 16 | 64 | 0.0625 | (1024) | 8 | 0.004 | (2048) |
| | (NRS22) | VISA | | | | | | | | | |
| 8 | HFH30495 | MRSA | II | + | 16 | 512 | 1 | (512) | 32 | 0.06 | (512) |
| 9^{f} | HIP13036 | MRSA/ | II | + | 8 | 256 | 1 | (256) | 8 | 0.004 | (2048) |
| | (NRS404) | VISA | | | | | | | | | |
| 10 | N315 | MRSA | II | + | 16 | 128 | 2 | (64) | 16 | 0.03 | (512) |
| | (NRS70) | | | | | | | | | | |
| 11 | 626 | MRSA | II | + | 16 | 512 | 4 | (128) | 32 | 0.5 | (64) |
| | (NRS382) | | | | | | | | | | |
| 12^{d} | NY-315 | MRSA | II | + | 16 | 256 | 8 | (32) | 32 | 0.13 | (256) |
| 13 | VRS5 | MRSA/ | II | + | 32 | 256 | 16 | (16) | 8 | 0.13 | (64) |
| | | VRSA | | | | | | | | | |
| 14^{f} | HIP07920 | MRSA/ | IV | + | 8 | 128 | 0.031 | (4096) | 8 | 0.016 | (512) |
| | (NRS21) | VISA | | | | | | | | | |
| 15^{d} | CA-127 | MRSA | IV | + | 16 | 64 | 1 | (64) | 4 | 0.13 | (32) |
| | (NRS643) | | | | | | | | | | |
| 16^{e} | HI049 | MRSA | IV | + | 16 | 128 | 2 | (64) | 8 | 0.06 | (128) |
| 17^{e} | FPR3757 | MRSA | IV | + | 16 | 64 | 2 | (32) | 4 | 0.004 | (1024) |
| 18^{d} | CT-19 | MRSA | IV | + | 16 | 256 | 4 | (32) | 4 | 0.13 | (32) |
| | (NRS676) | | | | | | | | | | |
| 19 | HIP133170 | MRSA/ | IV | + | 16 | 256 | 16 | (16) | 16 | 0.06 | (256) |
| | (VRS3a) | VRSA | | | | | | | | | |
| 20^{e} | HFH29568 | MRSA | IV | + | 16 | 64 | 4 | (16) | 16 | 0.06 | (256) |

^{a}SCC: Staphylococcus Chromosome Cassette. SCCmec types I, II, and IV produce PBP2a. SCCmec type II contains the intact mec operon producing MecR, MecI and PBP2a. SCCmec types I and IV contain only mecA expressing only PBP2a.
^{b}bla (+) strains carry the intact bla operon producing BlaR, BlaI and BlaZ (β-lactamase).
^{c}Fold reduction of MICs of OXA and MEM is shown in the parentheses.
^{d}USA600 clones.
^{e}USA300 clones.
^{f}Concentration of 4: 4 mg · L⁻¹.

The MICs of OXA and MEM were evaluated at a fixed concentration of 4 (8 mg·L⁻¹) for each strain, with the exception of NRS404 (Table 1, entry 9) and NRS21 strains (Table 1, entry 14) (4 mg·L⁻¹). Compound 4 had no effect on the OXA MIC and lowered MEM MIC by only 2-fold against strain NTCT8325, which does not harbor the bla nor mec operon (entry 1). The strains COL and JE2 are background strains of Staphylococcus Chromosome Cassette (SCC) mec type I and IV without the β-lactamase but produce PBP2a. However, they are not regulated by either MecR or BlaR. As expected, compound 4 showed minimal impact in reducing the MICs of OXA (2-fold) with these two strains (Table 1, entries 2 and 3). The MICs of MEM against these two strains were lowered by 16 and 64-fold (Table 1, entries 2-3), which is likely due to synergy of MEM with 4 (more on this later). Compound 4 lowered the MICs of MEM and OXA against MSSA strain ATCC29213 containing the bla operon by 2-4-fold, respectively (Table 1, entry 4). The tested MRSA strains were amongst three well-represented SCCmec types I, II, and IV that produce PBP2a. Compound 4 lowered the MICs of OXA and MEM by 64- and 512-fold, respectively, against strain NRS36, which belongs to SCCmec type I. Compound 4 reduced the MICs of OXA starting from 512-fold down to 16-fold (Table 1, entries 6-13) against SCCmec type II MRSA strains containing intact bla and mec operons. The MICs of MEM against SCCmec type II strains were lowered starting from 2048-fold down to 64-fold (Table 1, entries 6-13). Amongst SCCmec type IV strains that harbor only mecA and the intact bla operon, the MIC of OXA against strain NRS21 was reduced from 128 to 0.03 mg·L⁻¹ (4096-fold reduction: Table 1, entry 14). Against two MRSA strains that are resistant to vancomycin, compound 4 lowered the MIC of MEM by 64-fold (Table 1, entry 13) and 256-fold (Table 1, entry 19). Several MRSA strains of SCCmec type IV are clones of the notoriously virulent USA300 strains (Table 1, entries 3, 16, 17, and 20). Compound 4 lowered MICs of OXA and MEM against these USA300 strains starting from 256-fold down to 16-fold. Notably, compound 4 potentiated the activity of MEM across all 40 tested strains, lowering MICs to below resistant level, whereby MIC₉₀ values against MSSA are ≤0.12 mg-L.

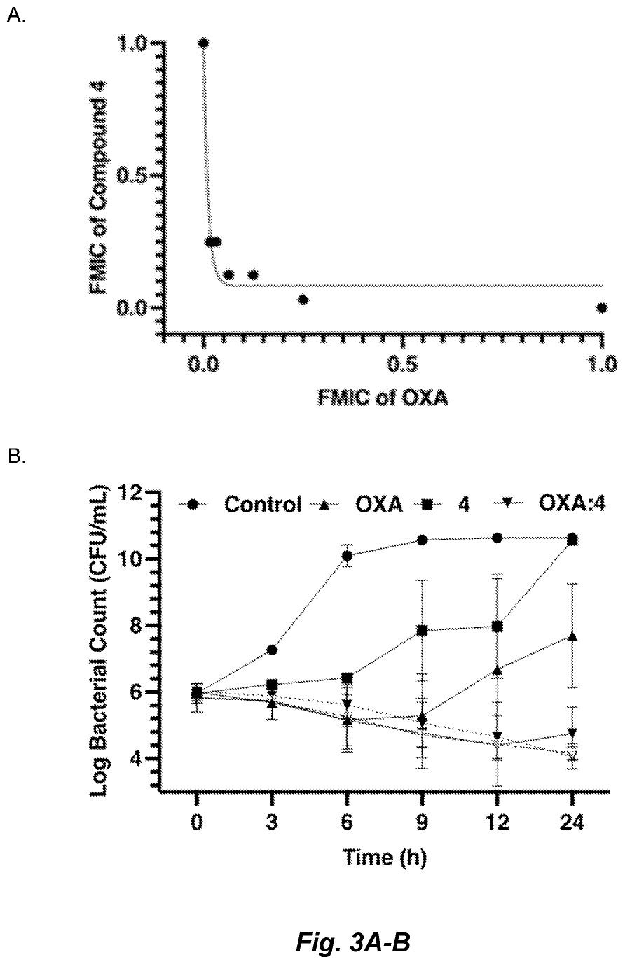

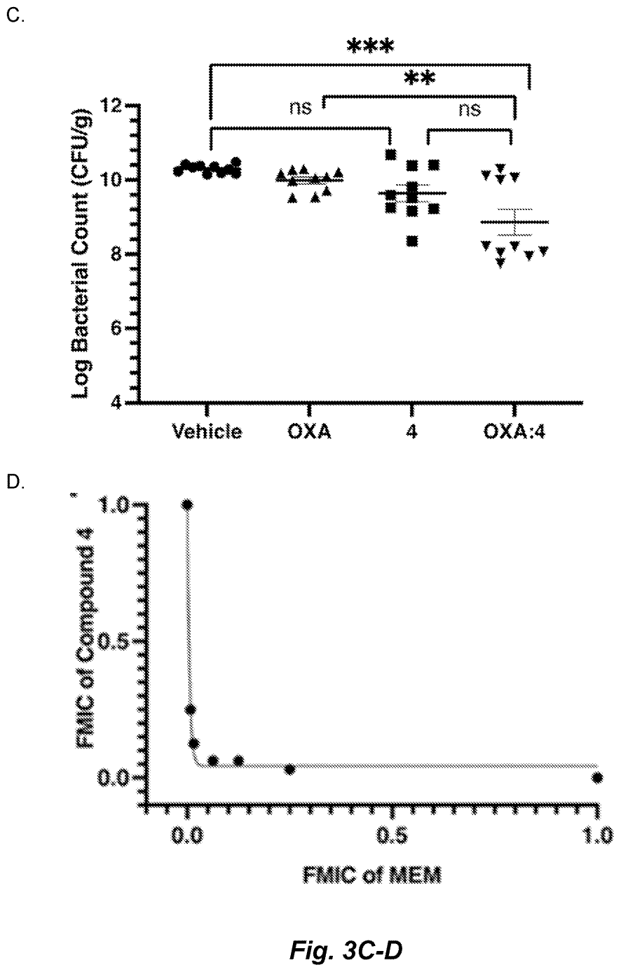

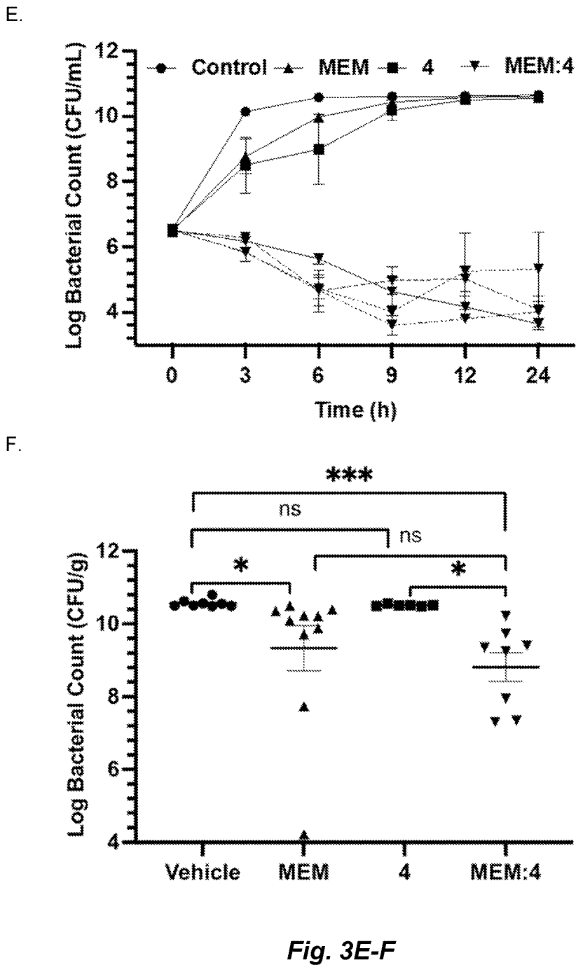

Synergy with β-Lactams and Time-Kill Assays. The highest potentiation for compound 4 was observed in the NRS21 strain with OXA and FOX, lowering MIC by 4096-fold and 2048-fold, respectively (Table 2, entry 25). The highest potentiation for compound 4 in a USA300 strain—the predominant MRSA strain that causes community-associated infections—was observed with MEM. Compound 4 was evaluated in the checkerboard assay with OXA in strain NRS21 (FIG. 3a), with MEM in strain FPR3757 (a USA300 strain, FIG. 3d), and with additional strains. In this assay, the antibiotics are compared individually and in combination. The fractional inhibitory concentration (FIC) index is calculated. Synergy is defined as an FIC value ≤0.5, indifference as FIC >0.5 to ≤4, and antagonism as FIC >4. With OXA in NRS21, the FIC index was 0.19, with FOX in NRS21 the FIC index was 0.25, and with MEM in FPR3757 the FIC index was 0.13, indicating that the combination of compound 4 with OXA or with MEM synergized strongly (decreased MIC). Compound 4 with OXA, MEM, FOX, or CPT also exhibit strong synergy against additional MRSA strains with FIC <0.25. With NRS21, we selected OXA over FOX because in the checkerboard assay, the FIC index was 0.19 with OXA, compared with 0.25 with FOX.

Time-kill assays, in which colony counts are obtained after individual and combination treatment as a function of time, confirmed synergy. In NRS21, the control in the absence of antibiotic resulted in 4.5-log increase in bacterial growth at 24 h, while compound 4 at 8 mg·L⁻¹ had a similar outcome (4.1-log increase, FIG. 3b). Compared to initial bacterial count at 0 h, OXA at 32 mg·L⁻¹ by itself had 1.9-log increase. The combination of OXA and 4 resulted in 1.7-log decrease for OXA:4 8:8 mg·L⁻¹, 1.7-log decrease for OXA:4 16:8 mg·L⁻¹, and 1.3-log decrease for OXA:4 32:8 mg·L⁻¹. At 24 h, the combination of OXA:4 decreased the bacterial growth by as much as 6.2-log (compared to the no-antibiotic control). The results indicated that a ratio of OXA:4 of 2:1 or 1:1 resulted in greater synergy (FIG. 3b).

Different combinations of MEM:4 (1:8, 2:4, and 2:8 mg·L⁻¹) with strain FPR3757 (USA300 clone) indicates a comparable 6.5-7.0-log decrease in bacterial growth in 24 h, when compared to the no-antibiotic control (FIG. 3e). Additional combinations of OXA:4 were studied in NRS21, NRS70, and NRS382, of MEM:4 in NRS21, NRS70, NRS404, HFH29568 (a USA300 strain), HFH30495 (a USA600 strain), of CPT:4 in NRS21 and NRS404.

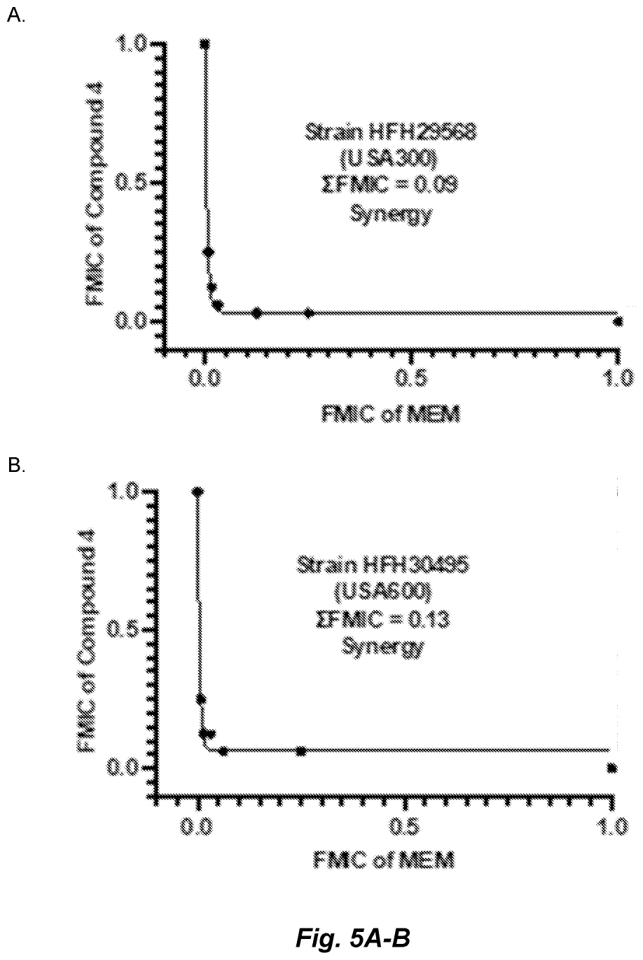

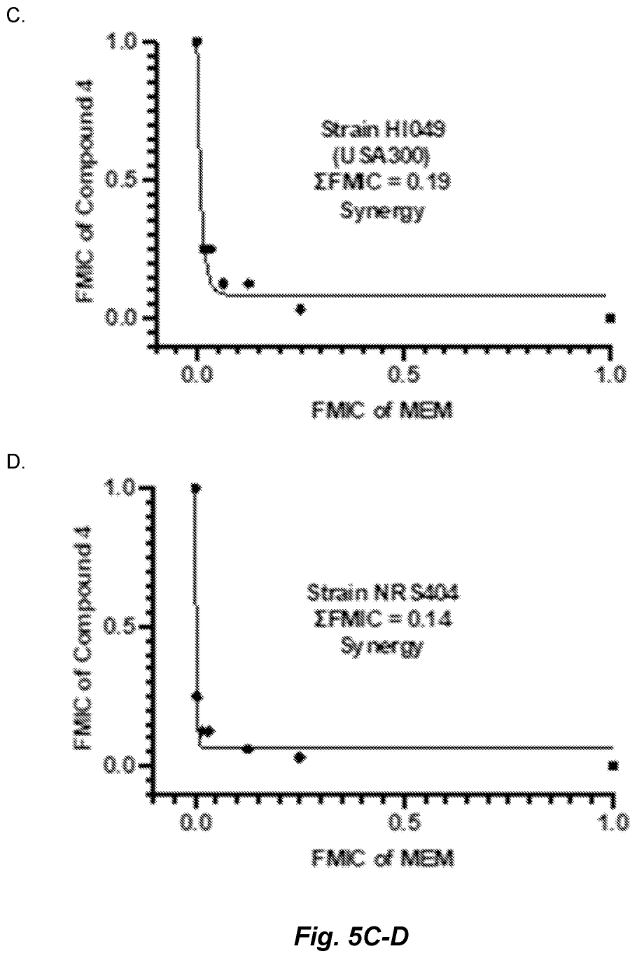

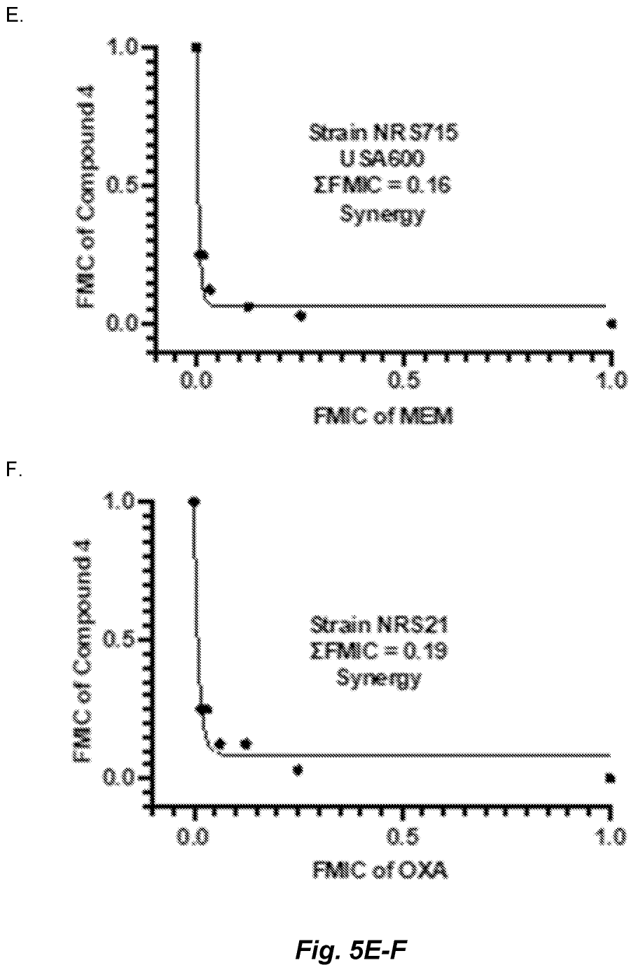

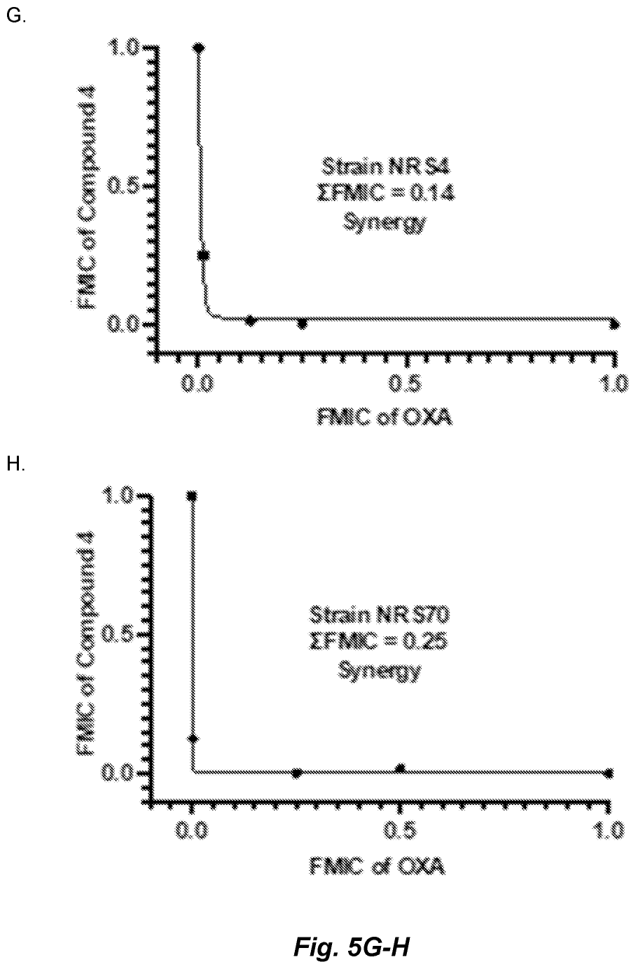

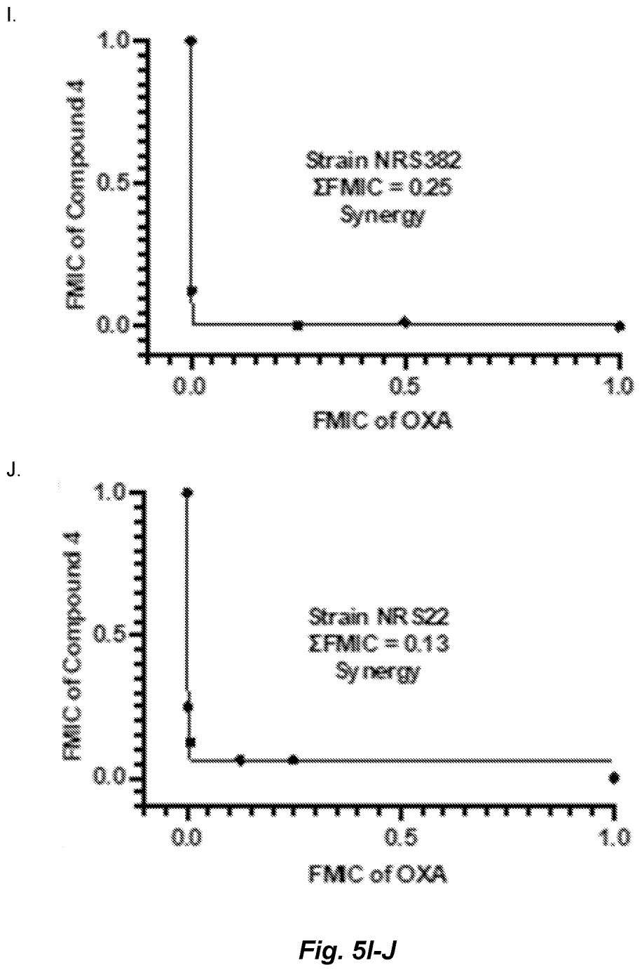

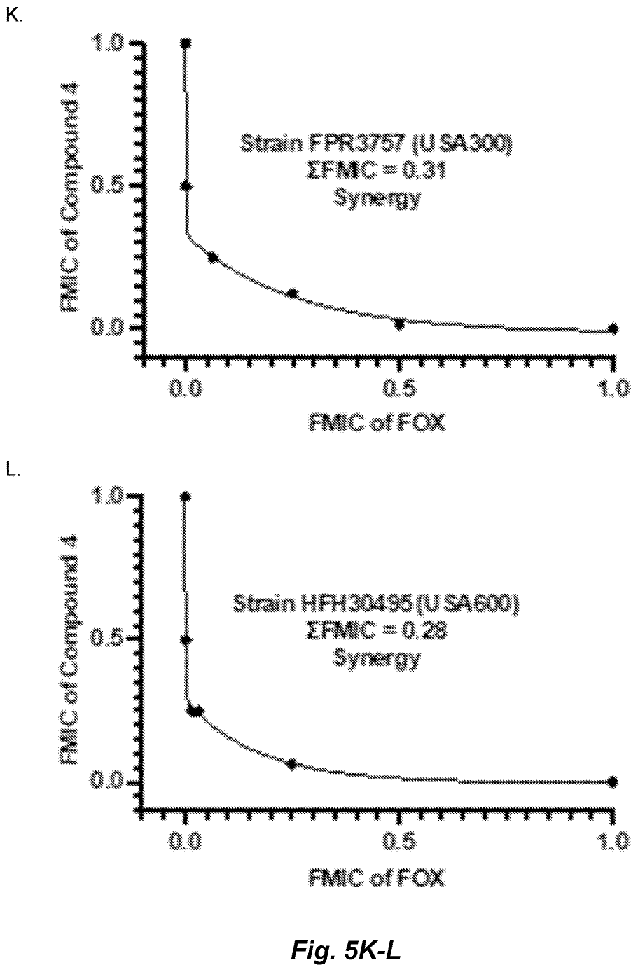

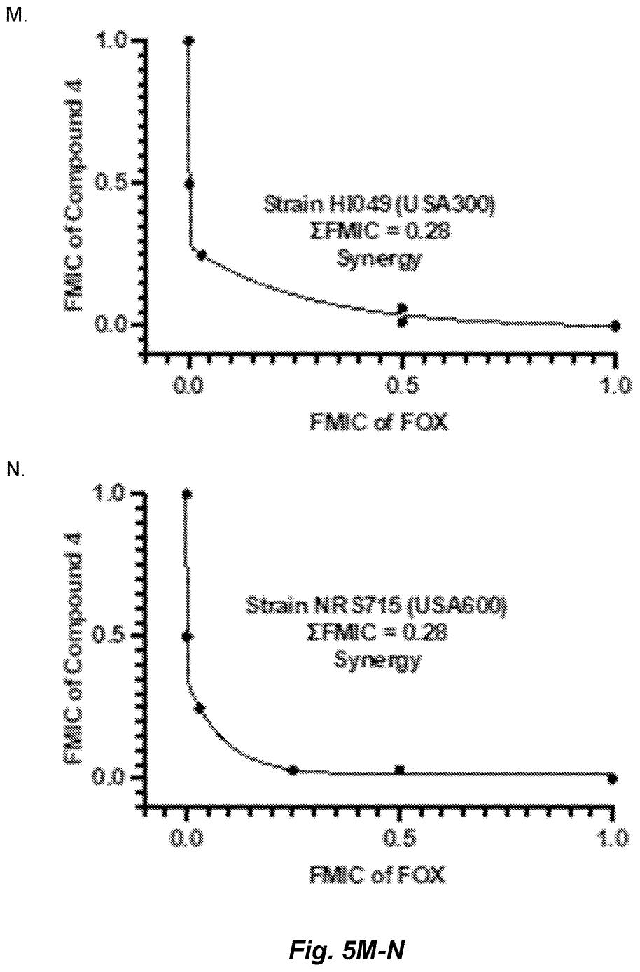

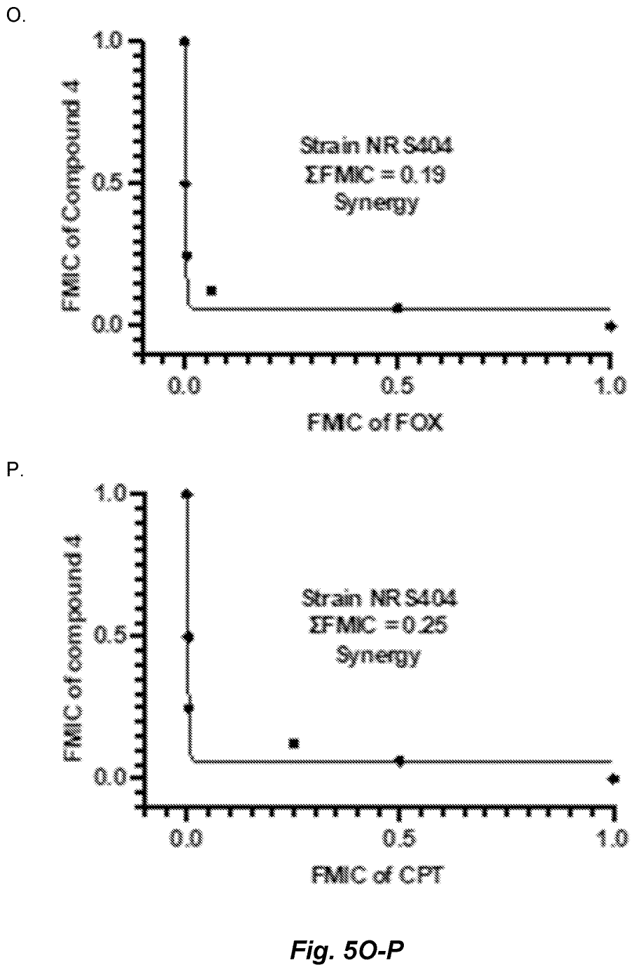

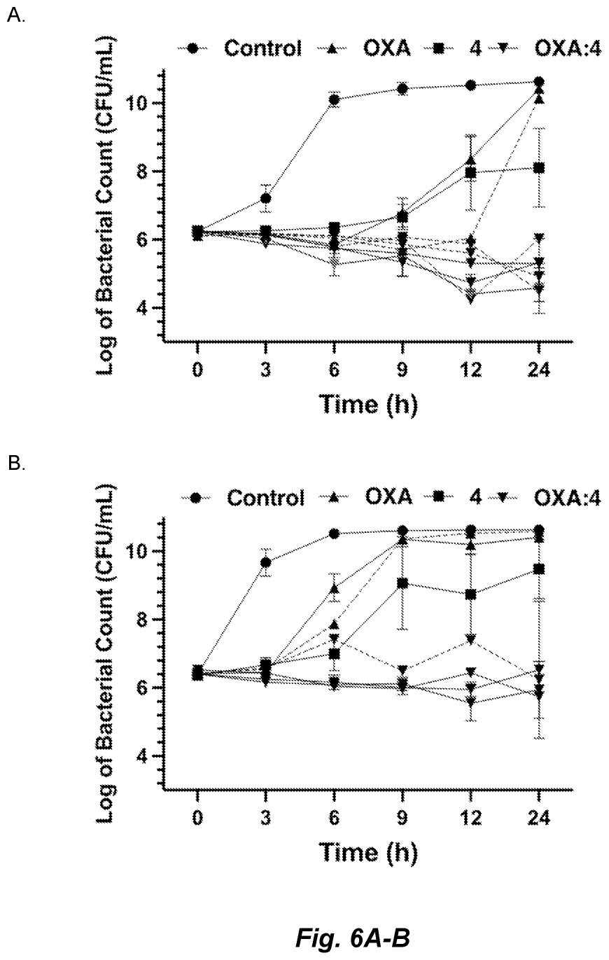

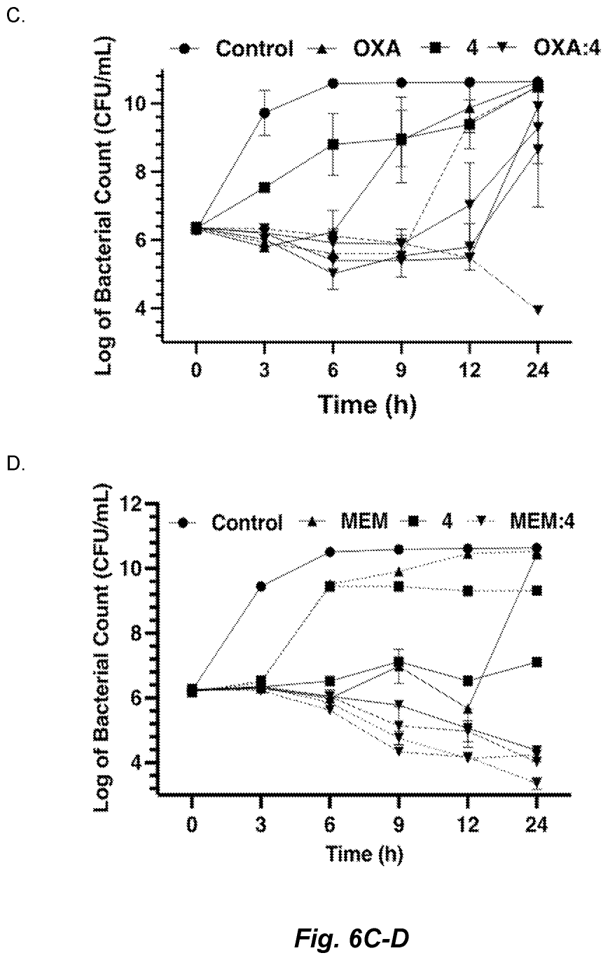

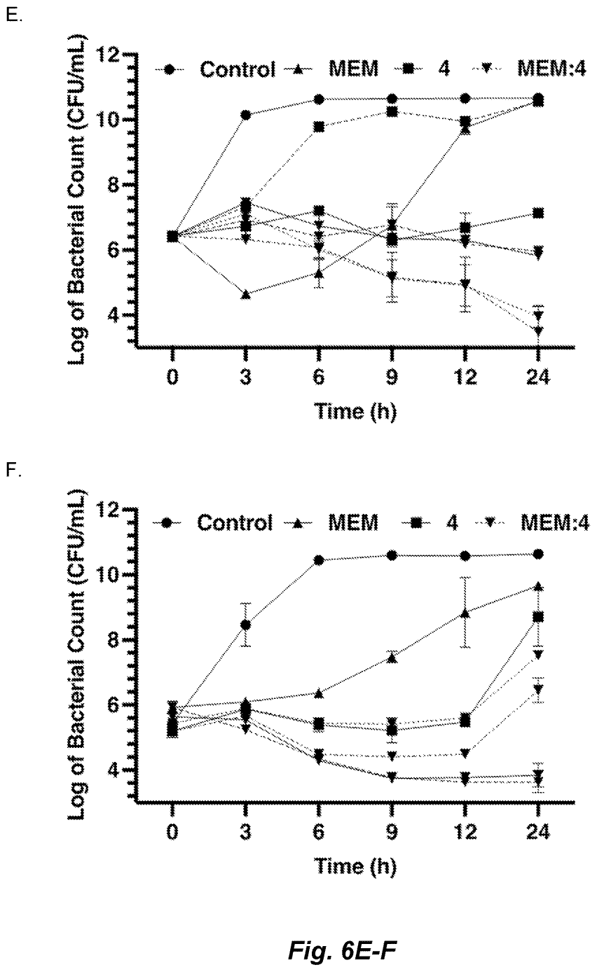

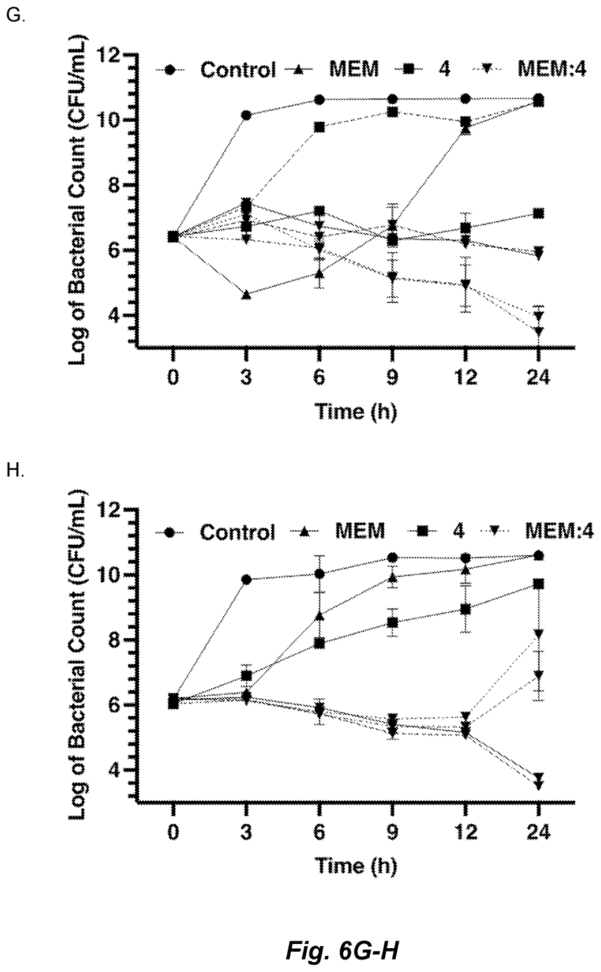

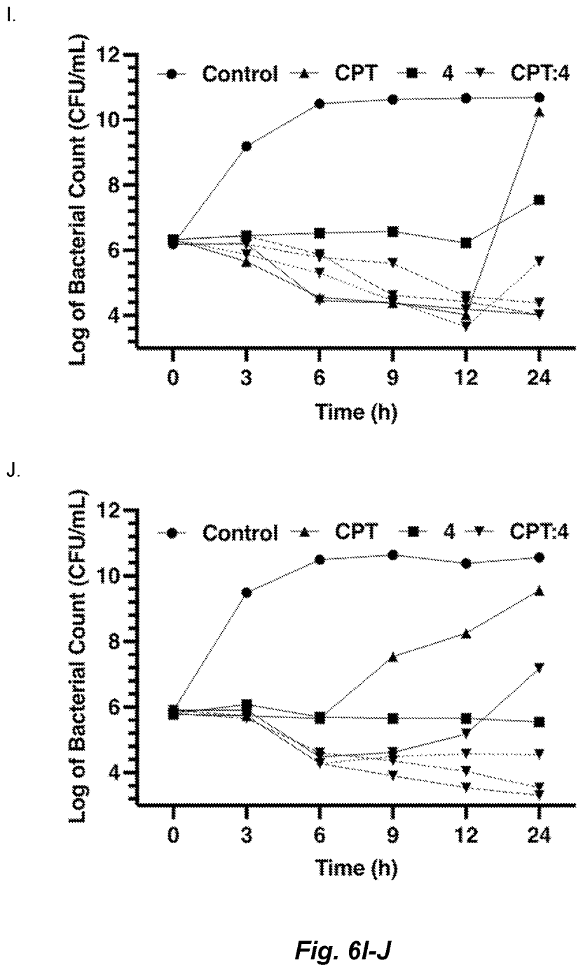

The best potentiation in a US300 strain (HI049, FPR3757, and HFH29568) was observed with MEM (Table 2, entries 27, 28, and 31). In the checkerboard, the FIC index was 0.19 for HI049 (FIG. 5c), 0.13 for FPR3757 (FIG. 3d), and 0.09 for HFH29568 (FIG. 5a). In the time-kill assay, MEM by itself had no activity in FPR3757 throughout the 24-h assay (FIG. 3e), while MEM by itself in HFH29568 had 2-log₁₀ decrease in bacterial growth at 6 h, then activity decreased thereafter and by 24 h there was no activity. We, thus, selected FPR3757 for animal studies.

We evaluated additionally the in vitro cytotoxicity of compound 4 using the XTT assay with HepG2 (liver) cells. Cell viability was 100% at 64 mg·L⁻¹. Compound 4 is not toxic to HepG2 cells.

TABLE 2
MICs of compound 4 and potentiation data of 4 with OXA, MEM, FOX and CPT against 40 MRSA and MSSA strains.

| | | | | MIC (mg · L⁻¹) | | | | | | | | | | | | |
|---|---|---|---|---|---|---|---|---|---|---|---|---|---|---|---|---|
| | | SCC | | | | 4 + | | | 4 + | | | 4 + | | | 4 + | |
| Entry | Strain | mec^{a} | bla^{b} | 4 | OXA | OXA^{c} | | MEM | MEM^{c} | | FOX | FOX | | CPT | CPT | |
| 1^{d} | NTCT | — | − | 16 | 0.25 | 0.25 | | 0.13 | 0.06 | (2) | 4 | 2 | (2) | 0.25 | 0.25 | |
| | 8325 | | | | | | | | | | | | | | | |
| | (NRS77) | | | | | | | | | | | | | | | |
| 2^{e} | COL | I | − | 32 | 512 | 256 | (2) | 16 | 1 | (16) | 256 | 32 | (4) | 1 | 1 | |
| | (NRS100) | | | | | | | | | | | | | | | |
| 3^{e} | JE2 | IV | − | 16 | 128 | 64 | (2) | 4 | 0.06 | (64) | 64 | 16 | (4) | 1 | 0.5 | (2) |
| 4^{d} | ATCC | — | + | 16 | 2 | 0.5 | (4) | 0.13 | 0.06 | (2) | 4 | 2 | (2) | 0.06 | 0.03 | (2) |
| | 29213 | | | | | | | | | | | | | | | |
| 5^{d} | MSSA476 | — | + | 32 | 0.5 | 0.13 | (4) | 0.13 | 0.03 | (4) | 4 | 2 | (2) | 0.13 | 0.06 | (2) |
| | (NRS72) | | | | | | | | | | | | | | | |
| 6^{d} | NRS128 | — | + | 16 | 0.5 | 0.13 | (4) | 0.13 | 0.06 | (2) | 4 | 2 | (2) | 0.06 | 0.03 | (2) |
| 7^{e} | LIM2 | I | + | 16 | 64 | 1 | (64) | 8 | <0.03 | (>256) | 128 | 8 | (16) | 1 | 0.5 | (2) |
| | (NRS36) | | | | | | | | | | | | | | | |
| 8^{e} | CM05 | I | + | 32 | 512 | 512 | | >32 | 2 | (16) | 512 | 8 | (64) | 2 | 2 | |
| 9^{e,f} | HIP5836 | II | + | 16 | 128 | 0.25 | (512) | 8 | <0.03 | (>256) | 64 | <0.5 | (>128) | 0.25 | 0.03 | (8) |
| | (NRS4) | | | | | | | | | | | | | | | |
| 10^{e,f} | HIP | II | + | 16 | 64 | <0.13 | (>512) | 8 | <0.03 | (>256) | 128 | <0.5 | (>256) | 0.25 | 0.25 | |
| | 07930 | | | | | | | | | | | | | | | |
| | (NRS22) | | | | | | | | | | | | | | | |
| 11^{e,h} | HFH | II | + | 16 | 512 | 1 | (512) | 32 | 0.06 | (512) | 256 | 1 | (256) | 1 | 0.5 | (2) |
| | 30495 | | | | | | | | | | | | | | | |
| 12^{e,f,j} | HIP13036 | II | + | 8 | 256 | 1 | (256) | 8 | <0.03 | (>256) | 256 | <0.5 | (>256) | 1 | 0.03 | (32) |
| | (NRS404) | | | | | | | | | | | | | | | |
| 13^{e} | N315 | II | + | 16 | 128 | 2 | (64) | 16 | <0.03 | (>512) | 128 | 8 | (16) | 1 | 0.5 | (2) |
| | (NRS70) | | | | | | | | | | | | | | | |
| 14^{e} | 626 | II | + | 16 | 512 | 4 | (128) | 32 | 0.5 | (64) | 512 | 64 | (8) | 1 | 0.5 | (2) |
| | (NRS382) | | | | | | | | | | | | | | | |
| 15^{e,h} | NY-315 | II | + | 16 | 256 | 8 | (32) | 32 | 0.13 | (256) | 256 | <0.5 | (>512) | 1 | 0.5 | (2) |
| | (NRS715) | | | | | | | | | | | | | | | |
| 16^{e,g} | HIP15178 | II | + | 32 | 256 | 16 | (16) | 8 | 0.13 | (64) | 128 | 16 | (8) | 1 | 0.5 | (2) |
| | (VRS5) | | | | | | | | | | | | | | | |
| 17^{e} | HIP06297 | II | + | 32 | 2 | <0.13 | (>16) | 4 | <0.03 | (>128) | 256 | 4 | (64) | 1 | 0.13 | (8) |
| | (NRS17) | | | | | | | | | | | | | | | |
| 18^{e} | M1712 | II | + | 16 | 256 | 16 | (16) | 16 | 0.13 | (128) | 128 | 16 | (8) | 1 | 0.5 | (2) |
| | (NRS127) | | | | | | | | | | | | | | | |
| 19^{e,h} | CA-347 | II | + | 16 | 256 | 4 | (64) | 16 | 0.25 | (64) | 128 | 2 | (64) | 1 | 0.5 | (2) |
| | (NRS648) | | | | | | | | | | | | | | | |
| 20^{e,h} | CO-23 | II | + | 32 | 256 | 128 | (2) | 32 | 0.5 | (64) | 128 | 8 | (16) | 1 | 1 | |
| | (NRS661) | | | | | | | | | | | | | | | |
| 21^{e,h} | MN-082 | II | + | 16 | 512 | 128 | (4) | 32 | 2 | (16) | 256 | 64 | (4) | 2 | 1 | (2) |
| | (NRS701) | | | | | | | | | | | | | | | |
| 22^{e,h} | TN-124 | II | + | 16 | 256 | 2 | (64) | 8 | 0.13 | (64) | 128 | 4 | (32) | 1 | 0.5 | (2) |
| | (NRS735) | | | | | | | | | | | | | | | |
| 23^{e,g} | AIS | II | + | 16 | 256 | 128 | (2) | 32 | 1 | (32) | 128 | 8 | (16) | 1 | 1 | |
| | 1000505 | | | | | | | | | | | | | | | |
| | (VRS10) | | | | | | | | | | | | | | | |
| 24^{e} | ATCC | III | + | 16 | 512 | 256 | (2) | 16 | 1 | (16) | 128 | 16 | (8) | 1 | 1 | |
| | 33591 | | | | | | | | | | | | | | | |
| 25^{e,f,j} | HIP07920 | IV | + | 8 | 128 | <0.13 | (>1024) | 8 | <0.03 | (>256) | 64 | <0.06 | (>1024) | 0.5 | <0.008 | (>64) |
| | (NRS21) | | | | | | | | | | | | | | | |
| 26^{e,h} | CA-127 | IV | + | 16 | 64 | 1 | (64) | 4 | 0.13 | (32) | 128 | 4 | (32) | 0.5 | 0.25 | (2) |
| | (NRS643) | | | | | | | | | | | | | | | |
| 27^{e,i} | HI049 | IV | + | 16 | 128 | 2 | (64) | 8 | 0.06 | (128) | 64 | 2 | (32) | 1 | 0.5 | (2) |
| 28^{e,i} | FPR3757 | IV | + | 16 | 64 | 2 | (32) | 4 | <0.03 | (>128) | 64 | 2 | (32) | 0.5 | 0.5 | |
| 29^{e,h} | CT-19 | IV | + | 16 | 256 | 4 | (32) | 4 | 0.13 | (32) | 64 | 4 | (16) | 1 | 0.5 | (2) |
| | (NRS676) | | | | | | | | | | | | | | | |
| 30^{e,g} | HIP | IV | + | 16 | 256 | 16 | (16) | 16 | 0.06 | (256) | 128 | 8 | (16) | 1 | 0.5 | (2) |
| | 133170 | | | | | | | | | | | | | | | |
| | (VRS3a) | | | | | | | | | | | | | | | |
| 31^{e,i} | HFH | IV | + | 16 | 64 | 4 | (16) | 16 | 0.06 | (256) | 32 | 2 | (16) | 1 | 1 | |
| | 29568 | | | | | | | | | | | | | | | |
| 32^{e} | LinR#12 | IV | + | 16 | 512 | 32 | (16) | 32 | 0.13 | (256) | 256 | 64 | (4) | 1 | 0.5 | (2) |
| | (NRS119) | | | | | | | | | | | | | | | |
| 33^{e} | MW2 | IV | + | 16 | 64 | 8 | (8) | 4 | 0.25 | (16) | 64 | 16 | (4) | 1 | 0.5 | (2) |
| | (NRS123) | | | | | | | | | | | | | | | |
| 34^{e,i} | USA300- | IV | + | 16 | 128 | 32 | (4) | 4 | 0.25 | (16) | 64 | 16 | (4) | 0.5 | 0.5 | |
| | 0114 | | | | | | | | | | | | | | | |
| | (NRS384) | | | | | | | | | | | | | | | |
| 35^{e} | HT | IV | + | 16 | 64 | 2 | (32) | 8 | 0.13 | (64) | 128 | 4 | (32) | 0.5 | 0.5 | |
| | 20020320 | | | | | | | | | | | | | | | |
| | (NRS245) | | | | | | | | | | | | | | | |
| 36^{e,h} | CA-263 | IV | + | 16 | 64 | 8 | (8) | 4 | 0.13 | (32) | 32 | 4 | (8) | 0.5 | 0.5 | |
| | (NRS647) | | | | | | | | | | | | | | | |
| 37^{e,h} | CO-34 | IV | + | 16 | 64 | 4 | (16) | 8 | 0.25 | (32) | 64 | 4 | (16) | 1 | 0.5 | (2) |
| | (NRS662) | | | | | | | | | | | | | | | |
| 38^{e,h} | SR4035 | IV | + | 16 | 128 | 16 | (8) | 16 | 0.25 | (64) | 128 | 8 | (16) | 1 | 0.5 | (2) |
| 39^{e} | 880 | IV | + | 16 | 256 | 32 | (8) | 16 | 0.25 | (64) | 64 | 2 | (32) | 1 | 0.5 | (2) |
| 40^{e} | H2138 | IV | + | 16 | 256 | 128 | (2) | 16 | 1 | (16) | 128 | 8 | (16) | 1 | 1 | |
| | (NRS271) | | | | | | | | | | | | | | | |

^{a}SCC: Staphylococcus Chromosome Casette. SCCmec types I, II, and IV produce PBP2a. SCCmec type II contains the intact mec operon producing MecR, MecI and PBP2a. SCCmec types I and IV contain only mecA expressing only PBP2a.
^{b}bla (+) strains carry the intact bla operon producing BlaR, BlaI and BlaZ (β-lactamase).
^{c}Fold reduction of MICs of OXA and MEM is shown in the parentheses.
^{d}MSSA strains.
^{e}MRSA strains.
^{f}Vancomycin-intermediate strain.
^{g}Vancomycin-resistant strain.
^{h}USA600 clones.
ⁱUSA300 clones.
^{j}Concentration of 4: 4 mg · L⁻¹.

Pharmacokinetics of Compound 4 and In Vivo Efficacy. We next evaluated the pharmacokinetics (PK) of compound 4 and its distribution to the thighs (sites of infection in the disease model) after a single subcutaneous dose at 25 mg·kg⁻¹ to mice. Maximum concentrations of 8.5 mg·L⁻¹ and 28 mg·L⁻¹ in plasma and thighs, respectively, were achieved at 4 h. The concentrations at 8 h were 3.9 mg·L⁻¹ in plasma and 20 mg·L⁻¹ in thighs, indicating that compound 4 distributed to the thighs. Based on these results, compound 4 can be given every 8 h.

The neutropenic thigh infection model in mice is commonly used to test efficacy of antibacterial agents. It is a highly standardized and reproducible assay that evaluates attenuation of bacterial load (and not animal survival) as the endpoint. All current clinical antibiotics are evaluated in this model. The direct injection of bacteria into the thighs of mice mimics a human soft-tissue infection. Prior to infection, mice are made neutropenic by injection of cyclophosphamide to cause the temporary depletion of neutrophils, dampening the immune response and making mice more susceptible to infection. Based on the PK properties of compound 4 and the highest bacterial count reduction in the time-kill assay of OXA and MEM in combinations with compound 4, the mouse neutropenic thigh infection model was performed with the NRS21 and FPR3757 strains. Infected mice treated with 4 or OXA did not show a statistically significant decrease in bacterial counts compared to vehicle-treated mice (FIG. 3c) at 24 h. In the OXA (40 mg·kg⁻¹):4 (20 mg·kg⁻¹) combination, 1.7-log reduction in bacterial growth compared to vehicle was observed (p<0.001). A 1.8-log reduction was observed in the MEM:4 combination MEM (50 mg·kg⁻¹):4 (20 mg·kg⁻¹) compared to vehicle, (p<0.001, FIG. 3f), 1.2-log reduction between vehicle and MEM (p<0.05), 0.06-log reduction between vehicle and 4 (p>0.05), and 1.7-log reduction between 4 and MEM:4 (p<0.001, FIG. 3f). Additional combinations of 4 and MEM or OXA were also tested. The potentiation of activity of β-lactam antibiotics by compound 4 observed in vitro, translates to in vivo activity.

### Discussion.

We have described in this report inhibition of the β-lactam sensor/signal transducer BlaR as a viable strategy to resurrect the use of β-lactam antibiotics from obsolescence in treatment of S. aureus infections. We have identified a class of inhibitors that binds to the sensor domain of BlaR, whereby they prevent the signal-transduction step that precipitates manifestation of the resistance phenotype. Since the resistance mechanism is no longer triggered by the inhibited BlaR, the organism experiences restoration of susceptibility to β-lactam antibiotics. Potentiation of the anti-S. aureus activity of several β-lactam antibiotics was investigated, which can be as much as 4096-fold. The initial phase of the discovery was in silico, followed by refinement of the hits by synthesis and further screening. The lead compound 4 synergizes well with β-lactam antibiotics, shows modest inhibitory properties against active-site serine penicillin-binding proteins and exhibits in vivo efficacy in a mouse thigh-infection model.

We note that two cyclic boronates, vaborbactam and taniborbactam, as β-lactamase inhibitors have advanced. The former is presently a clinical agent in combination with β-lactams and the latter is in clinical trials. These two compounds exhibited no activity in potentiation of the anti-S. aureus activities of MEM, OXA, FOX and CPT, underscoring the specificities of compound 4 for BlaR, per the design paradigms. The Centers for Disease Control and Prevention classify MRSA as a serious threat, which resulted in 323,700 hospitalizations in the United States in 2017 and 10,600 deaths. Compound 4 hold the promise of addressing the unmet clinical need in treatment of infections by difficult S. aureus strains.

### Pharmaceutical Formulations.

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard or soft shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, see U.S. Pat. No. 4,992,478 (Geria), U.S. Pat. No. 4,820,508 (Wortzman), U.S. Pat. No. 4,608,392 (Jacquet et al.), and U.S. Pat. No. 4,559,157 (Smith et al.). Such dermatological compositions can be used in combinations with the compounds described herein where an ingredient of such compositions can optionally be replaced by a compound described herein, or a compound described herein can be added to the composition.

Useful dosages of the compounds described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The invention provides therapeutic methods of treating an infection in a mammal, which involve administering to a mammal having cancer an effective amount of a compound or composition described herein. A mammal includes a primate, human, rodent, canine, feline, bovine, ovine, equine, swine, caprine, bovine and the like.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. General Methods

Antibiotics. MEM was purchased from US Pharmaceuticals (Pittsburgh, PA). FOX was purchased from Sigma-Aldrich (St. Louis, MO). OXA was purchased from TCI Chemicals (Portland, OR). Commercially available CPT fosamil was dephosphorylated using alkaline phosphatase, as previously described (Proc. Natl. Acad. Sci. U.S.A. 110, 16808-13 (2013)).

Bacterial strains. The 40 MSSA, MRSA, VRSA and VISA strains were collected from various sources over many years, including from BEI Resources (Manassas, VA). ATCC 29213 was purchased from American Type Culture Collection (Manassas, VA).

Construction of the fluorescent-reporter systems. In order to create a green-fluorescent reporter, the blaR/I segment including its promoter region was first synthesized by Genscript (Piscataway, NJ) based on the sequence of the bla operon in the staphylococcal plasmid pI258. The synthetic blaR/I DNA had two restriction enzyme sites: SphI at the 5′-end and EcoRI at the 3′-end. The SphI/EcoRI-digested blaR/I was ligated with the Escherichia coli/Staphylococcus aureus-shuttle vector pCN52, which harbors a gfp gene without its promoter sequence, at the same restriction-enzyme sites. The resulting plasmid pCN52RI was inserted into E. coli DH5α. The sequences of the blaR/I, the promoter region and the gfp were confirmed by DNA sequencing.

The gfp in pCN52RI was replaced by the venus or the mCherry genes to generate a yellow-fluorescent reporter and a red-fluorescent reporter, respectively, as follows. The venus and the mCherry genes were amplified by PCR using Q5 DNA polymerase (New England Biolabs, Ipswich, MA) from the plasmids pTH3 and pRN11, respectively, purchased from Addgene (Watertown, MA). Two sets of primers were used: CK393 (5′-GTTAACTAATTAATTTAAGAAGGAGATATAAATATGAGTAAAGGAGAAGAACTTTTCAC-3′) (SEQ ID NO: 1) and CK394 (5′-ATTTAGAATAGGCGCGCCCTTACTTGTACAGCTCGTCCATG-3′) (SEQ ID NO: 2) for the venus; CK389 (5′-GCATTTAGAATAGGCGCGCCCTTACTTGTACAGCTCGTCCATG-3′) and (SEQ ID NO: 3) CK392 (5′-GTTAACTAATTAATTTAAGAAGGAGATATAAATATGGTGAGCAAGGGCG-3′) (SEQ ID NO: 4) for the mCherry. The vector pCN52RI excluding the gfp was also amplified with the primers, CK386 (5′-GGCGCGCCTATTCTAAATGC-3′) (SEQ ID NO: 5) and CK387 (5′-ATCTCCTTCTTAAATTAATTAGTTAACGAATTCGATATTAC-3′) (SEQ ID NO: 6). Each PCR product of the venus and the mCherry genes was ligated with the PCR product of pCN52RI using an NEBuilder HiFi DNA Assembly kit (New England Biolabs, Ipswich, MA). E. coli DH5α was transformed by each of the resulting plasmids, pCN52RI-venus and pCN52RI-mCherry. The plasmid sequences were confirmed by sequencing (Plasmidsaurus, Eugene, OR). Each fluorescent-reporter plasmid was electroporated into S. aureus NRS144 using the Gene Pulser Xcell Electroporator (Bio-Rad, Hercules, CA) for evaluation of the expression of the fluorescent protein in the presence of a β-lactam inducer (FIG. 4).

Minimum-inhibitory concentration (MIC) determination and potentiation assay of compound 4 with OXA, MEM, FOX, and CPT. MICs were determined by the microdilution broth method following the Clinical & Laboratory Standards Institute (CLSI) guidelines. Briefly, determination of MICs was performed by two-fold serial dilutions of each compound in cation-adjusted Mueller-Hinton II broth (without or with 2% NaCl for Staphylococcus aureus when tested with OXA). Experiments were performed in triplicate in 96-well plates with 5×10⁵ CFU·mL⁻¹ of bacteria. The plates were incubated at 37° C. for 18 h or 24 h when tested with OXA. The MIC was the concentration for which bacterial growth was not observed.

Fluorescent assay for screening of BlaR potential inhibitors. A 3-mL portion of lysogeny broth (LB) medium was inoculated with NRS144pCN52RI Venus from frozen stocks and grown overnight at 37° C. The overnight culture was then diluted 1:150 fold with fresh LB containing 50 mg/mL of erythromycin and incubated at 37° C. to an OD₆₀₀ of 0.3, at which point fluorescence was induced by the addition of CBAP to a final concentration of 10 μg·mL⁻¹. The culture was aliquoted (99 L) into 96-well plate and 1 L of inhibitor solution (1 mg·mL⁻¹ in DMSO) was added to each well. Negative control (DMSO without CBAP) and positive control (CBAP without inhibitor) were included. The plate was incubated at 30° C. for 2 h with intermittent shaking. Fluorescence and OD₆₀₀ measurements were taken by a Cytation-5 multimode microplate reader. Percent inhibition of BlaR and bacterial growth were calculated using absorbance unit (AU) and OD₆₀₀ values of each sample entered into this formula:

$$\% BlaR inhibition = \frac{{AU}_{sample} - {AU}_{DMSO}}{{AU}_{CBAP} - {AU}_{DMSO}} \times 100 \%$$

$$\% bacterial growth inhibition = \frac{{OD}_{sample} - {OD}_{DMSO}}{{OD}_{CBAP} - {OD}_{DMSO}} \times 100 \%$$

Structural resolution: X-ray crystallography. A triple mutant (K369A, K370A, K372A) BlaR construction of the sensor domain (332-583) was used for the crystallographic studies. The surface mutations improved crystal growth. Crystals were obtained at 6-8 mg·mL⁻¹ using the sitting-drop vapor diffusion method at 18° C. and cryoprotected using 20% glycerol. High-throughput crystallographic screening identified 0.085 M sodium cacodylate pH 5.2-6.3, 21-31% PEG 8K, and 15% glycerol as the best conditions for obtaining crystals with both apo- and co-crystallized BlaR. The BlaR:2 complex was obtained by co-crystallization with an excess of the inhibitor and the cryo-protectant solution. The best data set for the BlaR:4 complex was obtained by soaking apo crystals with 4 mM of the inhibitor. The BlaR-cefepime structure was obtained by soaking apo-crystals with 1 mM of the antibiotic for 72 h.

Data collection, phasing, and model refinement. Diffraction data sets were collected at the ALBA synchrotron in XALOC beamline using a Pilatus 6M detector. The collected datasets were processed with XDS and Aimless. Crystals belonging to the P2₁ space group. The complexes diffract at 1.97 resolution for 4, 2.00 Å for 2, and 2.52 Å for cefepime. In all structures two protein molecules were found in the asymmetric unit showing a 48% solvent content. The structures were solved by molecular replacement using the apo-BlaR coordinates (PDB 1XA7) as the search model. The model was manually completed using Coot and refined by using PHENIX and REFMAC5.

Checkerboard assay. The synergistic interactions of compound 4 with OXA, MEM, FOX and CPT were tested on MRSA strains using the checkerboard assay in 96-well plates. The final inoculum in each well was 5×10⁵ CFU·mL⁻¹, and the result was read after 24-h incubation at 37° C. The fractional inhibitory concentration (FIC) index was calculated for each combination. An FIC index of ≤0.5 is considered synergistic. The experiment was done in triplicates.

Time-kill assay. MRSA strains (NRS21, NRS70, NRS382, NRS404, HFH 29568, and HFH 30495) were grown overnight on tryptic soy agar plate. Bacteria were diluted in Muller-Hinton broth containing 2% NaCl to 108 CFU·mL⁻¹. For the assays, 5 mL of cation-adjusted Muller-Hinton broth II supplemented with 2% NaCl containing 10⁶ CFU·mL⁻¹ of MRSA and different concentrations of compound 4 and either OXA, MEM or CPT were incubated at 37° C. For MEM and CPT, cation-adjusted Muller-Hinton broth without 2% NaCl was used. Aliquots were taken at 0, 3, 6, 9, 12, and 24 h and were diluted serially for bacterial counts. The assay was done in triplicate.

XTT assay. Compound 4 was evaluated in triplicate in the XTT assay (XTT cell proliferation assay, Canvax, Spain) with HepG2 cells, as described previously (Eur. J. Med. Chem. 253, 115329 (2023)). GraphPad Prism 5 (San Diego, CA) was used to calculate IC₅₀ values.

Animals. Outbred 8-week-old female ICR mice for PK and neutropenic thigh infection studies were purchased from Envigo, Indianapolis, IN. The average weight of the mice for the PK study was 30 g (range 26.1 to 33.1 g); mice in the neutropenic thigh infection weights ranged from 24.2 to 37.7 g and were on average 29 g at the initiation of the study. Mice were housed in polycarbonate shoebox cages (for the PK study) or in disposable cages (for the infection studies) with ¼-inch corncob (The Andersons Inc., Maumee, OH) and Alpha-Dri (Shepherd Specialty Papers, Inc., Richland, MI) bedding with a 12-h light/dark cycle at 72° F. The animals were given food (Teklad 2918 irradiated extruded rodent diet) and water ad libitum. The food and water were autoclaved for the infection studies. All procedures were performed in accordance with and approved by the University of Notre Dame Institutional Animal Care and Use Committee.

PK study. Mice (n=15) were given a single subcutaneous dose of compound 4 at 25 mg·kg⁻¹ (100 μL of 7.5 mg-mL-1 solution in 20% DMSO/20% Tween-80/60% sterilized water). At 0.5, 1, 2, 4, and 8 h, mice were euthanized. Terminal blood was collected by cardiac puncture into heparinized (sodium heparin, Sigma-Aldrich, St. Louis, MO) syringes, and centrifuged (20,000×g at 4° C. for 15 min) to obtain plasma. A 60-μL aliquot of plasma was mixed with 60 μL of a 10 μg·mL⁻¹ solution of internal standard in acetonitrile. After centrifugation (17,000×g for 10 min), a 60-μL of the supernatant was taken for ultraperformance liquid chromatography (UPLC) analysis with UV detection.

Thighs were excised, weighed, and mixed with 100 μL of 10 μg·mL⁻¹ of internal standard in acetonitrile. Acetonitrile (900 μL) was added, and the mixture was homogenized on a Bullet blender (Next Advance, Inc., Averill Park, NY). After centrifugation at 17,000×g for 10 min, the supernatant was collected. The extraction was repeated two additional times. The combined supernatants were concentrated on a speed vac. The residue was dissolved in 100 μL of acetonitrile, and the mixture was centrifuged. A 60-μL aliquot of the supernatant was placed in an autosampler vial.

Plasma and thigh samples were analyzed on a Waters Acquity UPLC (Waters Corp., Milford, MA) coupled with a diode array detector with UV detection at 320-330 nm. A C18 Atlantis™ T3 column 2.1 i.d.×100 mm length 3 μm-particle size column was used at a flow rate of 0.5 mL/min. The gradient consisted of 0.5-min from 80% A/20% B to 65% A/35% B, 4.5-min to 25% A/75% B, 0.5-min to 65% A/35% B, 2-min to 80% A/20% B, where A=0.1% formic acid in water and B=0.1% formic acid in acetonitrile. Internal standard and compound 4 eluted at 4.2 and 4.6 min, respectively.

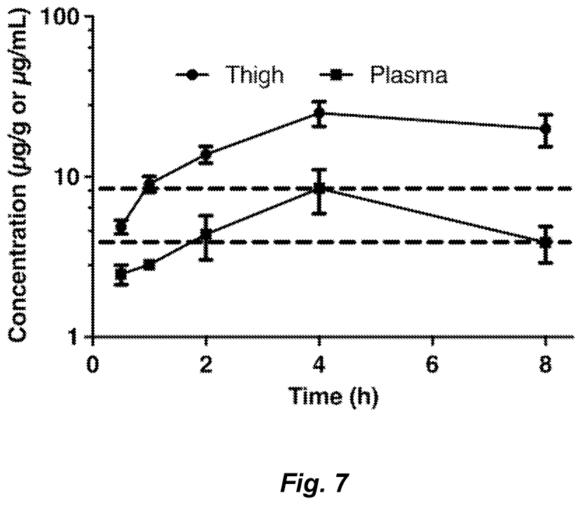

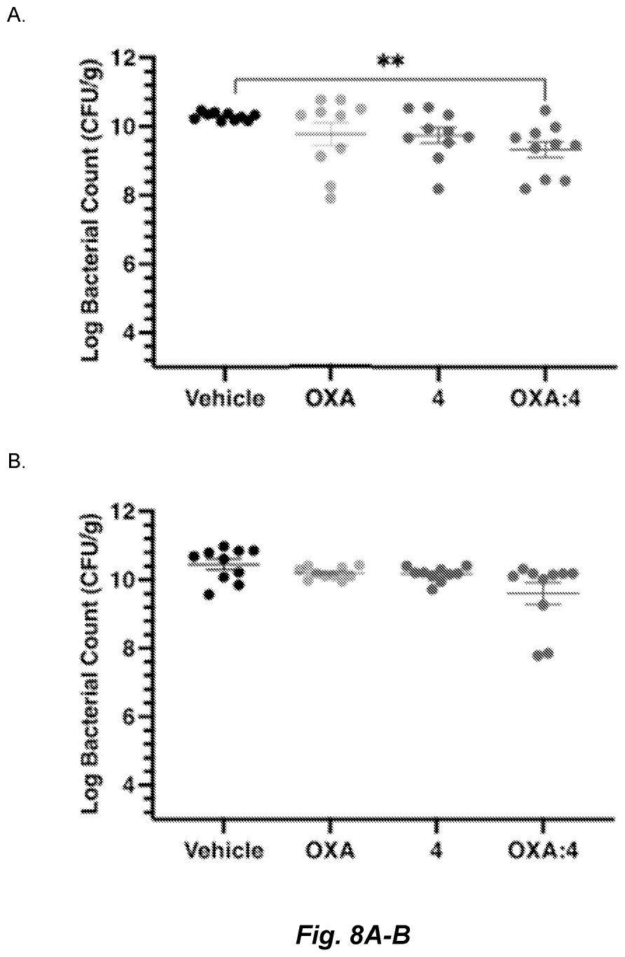

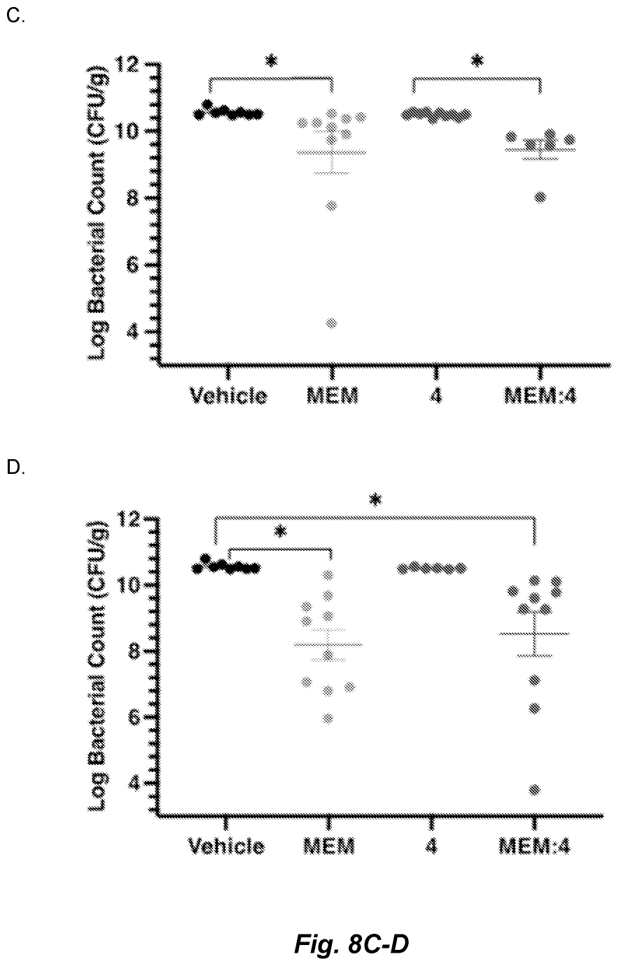

Calibration curves containing known concentrations of compound 4 (ranging from 0 to 200 μg·g⁻¹ tissue for thighs or 0 to 40 μg·mL⁻¹ for plasma) and internal standard in blank control thigh tissue or control plasma were prepared. Concentrations in the thighs and plasma were determined from the calibration curve regression parameters relative to internal standard. Results are shown in FIG. 7.

Neutropenic thigh infection studies. Mice (n=5 per group, total of 155 mice) were made neutropenic by injection of cyclophosphamide (Sigma-Aldrich, St. Louis, MO) intraperitoneally at 200 mg·kg⁻¹ (100 μL of a 60 mg·mL⁻¹ solution) on days −4 and −1. MRSA NRS21 and FPR3757 were grown in Tryptic Soy Agar and diluted to a final concentration of 10⁶ CFU·mL⁻¹ in Brain-heart Infusion. On day 0, 100 μL of bacterial inoculum was injected intramuscularly into each of the right and left thighs. At 1 h after infection, the mice received subcutaneous injections of compound 4 (at 10, 15, 20, or 30 mg·kg⁻¹), OXA (sodium oxacillin, TCI Chemicals, Portland, OR, at 20, 30, or 40 mg·kg⁻¹) or combinations of OXA:4. MEM was given intraperitoneally at 50 or 100 mg·kg⁻¹. OXA and compound 4 were given subcutaneously at 1 h, 9 h and 17 h after infection. MEM was given intraperitoneally at 50 or 100 mg·kg⁻¹ at 1 h, 5 h, 9 h, 13 h, 17 h, and 21 h after infection. Compound 4 and OXA were prepared in 20% DMSO/20% Tween-80/60% sterilized water. MEM was prepared in 10% DMSO/20% Tween80/70% saline (0.9% NaCl in sterilized water). A vehicle group was included. At 24 h after infection, the mice were euthanized. Both thighs were dissected aseptically, weighed, and homogenized with two volumes of 1×PBS in a Bullet blender (Next Advance, Inc., Averill Park, NY) at 4° C., followed by centrifugation. A 20-μL aliquot of the supernatant was transferred to 180 μL of 0.1% agar saline, and serially diluted up to 10-8, and plated in triplicate. The number of colonies were counted.

Statistical analysis. Statistical analysis for the neutropenic thigh infection studies was performed using GraphPad Prism Version 10 with a mixed model fitting a mixed model. The Tukey's multiple comparisons test was used for group comparison and to compute the adjusted p values. One-way and two-way ANOVA was used for the time-kill assays.

Abbreviations. CBAP, 2-(2-carboxyphenyl)-benzoyl-6-aminopenicillanic acid; CPT, ceftaroline; FIC, fractional inhibitory concentration; FIC, fractional inhibitory concentration; FOX, cefoxitin; GFP, green fluorescent protein; MEM, meropenem; MIC, minimal inhibitory concentration; MSSA, methicillin-sensitive Staphylococcus aureus; MRSA, methicillin-resistant Staphylococcus aureus; OD, optical density; OXA, oxacillin; PBP, penicillin-binding protein; SCC, Staphylococcus Chromosome Cassette; VISA, vancomycin-intermediate Staphylococcus aureus; VRSA, vancomycin-resistant Staphylococcus aureus.

### Example 2. Synthetic Methods

General. Commercially available chemicals and anhydrous solvents were purchased from Millipore-Sigma, TCI Chemicals, and VWR and were used without further purification. Column chromatography used a Teledyne ISCO CombiFlash Rf 200i. TLC used Sigma precoated aluminum-backed TLC plates (SIL G/UV254) visualized using a UV lamp, p-anisaldehyde staining, or KMnO₄ staining. The purity of all final compounds was ≥95% as assessed by LC/MS analysis using a Bruker microOTOF-QII instrument. NMR spectra were recorded at room temperature on a Bruker-400 spectrometer. ¹⁹F-NMR chemical shift was in reference to hexafluorobenzene signal in DMSO-d₆ and CD₃CN at −162.4 ppm and −164.4, respectively. Fluorescence and OD₆₀₀ values were recorded using a Cytation 5 multimode microplate reader at room temperature.

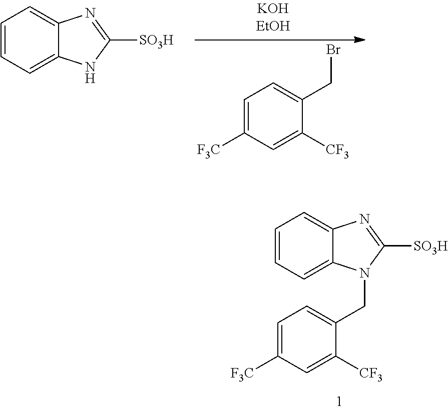 [SMILES: O=S(=O)(O)c1nc2ccccc2[nH]1 ; FC(F)(F)c1ccc(CBr)c(C(F)(F)F)c1 ; O=S(=O)(O)c1nc2ccccc2n1Cc1ccc(C(F)(F)F)cc1C(F)(F)F]

1-[[2,4-Bis(trifluoromethyl)phenyl]methyl]benzimidazole-2-sulfonic acid (1). Benzimidazole-2-sulfonic acid (0.50 g. 2.5 mmol) was dissolved into a mixture of 2 M KOH/ethanol (40 mL, 1:1 v/v ratio) at room temperature in a 100 mL round-bottom flask equipped with a magnetic stir bar. A portion of 2,4-bis(trifluoromethyl)benzylbromide (0.2 mL, 2.75 mL) was added slowly to the reaction mixture. The reaction was stirred overnight at room temperature. After 16 h, the reaction was acidified with 3 N HCl to pH 1.0. The white precipitate was filtered, washed with water (20 mL) and diethyl ether (20 mL×2), and then kept under high vacuum overnight to give the title compound (0.79 g, 75% yield): ¹H NMR (400 MHz, DMSO-d₆) δ ppm 9.5 (br s, 1H), 8.17 (1H, d, J=1.9 Hz), 7.92 (1H, dd, J=8.4 Hz, 1.9 Hz), 7.84 (1H, d, J=8.1 Hz), 7.61 (2H, m), 7.55 (1H, s), 6.93 (1H, d, J=8.4 Hz), 6.24 (2H, s); ¹³C NMR (101 MHz, DMSO-d₆) δ ppm 153.4, 138.4, 132.7, 131.1, 130.5, 129.3 (q, ²J_{CF}=33 Hz), 128.7, 127.4, 127.3 (q, ²J_{CF}=31 Hz), 127.2 (q, ³J_{CF}=2.2 Hz), 124.1 (q, ¹J_{CF}=274 Hz), 124.1 (q, ¹J_{CF}=274 Hz), 123.8 (dq, ³J_{CF}=2.2 Hz), 116.5, 113.7, 45.6; ¹⁹F NMR (376 MHz, DMSO-d₆) δ ppm −59.47, −61.21; MS (m/z) [M+H]⁺, calcd for C₁₆H₁₁F₆N₂O₃S, 425.0395, found 425.0398.

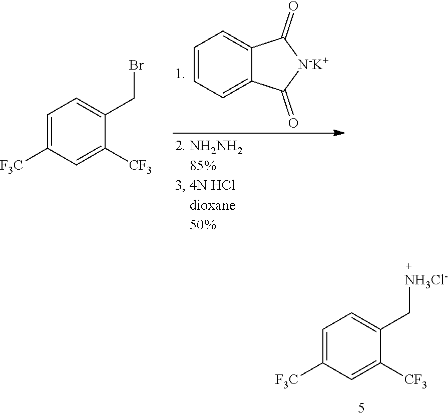

1-Aminomethyl-2,4-Bis(trifluoromethyl)benzene-HCl (5). Potassium phthalimide (4.0 g, 21.6 mmol, 1.05 equiv) was added into a 50 mL two-neck round-bottom flask equipped with a magnetic stir bar, followed by the addition of 15 mL anhydrous N,N-dimethylformamide (DMF). Subsequently, 2,4-bis(trifluoromethyl)benzylbromide (3.8 mL, 6.28 g, 20.5 mmol, 1.0 equiv) was added to the reaction mixture at room temperature. The mixture was stirred at 60° C. for 12 h. The reaction mixture was cooled to room temperature and poured into a 250 mL beaker containing 100 mL ice water to form a white precipitate. The precipitate was recovered by filtration, crushed into fine powder, resuspended in deionized water. The white powder was recovered by filtration, washed with deionized water (20 mL×2) and pentane (30 mL), and dried in vacuo overnight to give the N-[2,4-bis(trifluoromethyl)benzyl]phthalimide (7.33 g, 96%) as a white crystalline solid, which was used without further purification.

The N-[2,4-bis(trifluoromethyl)benzyl]phthalimide (3.7 g, 10 mmol) was dissolved in anhydrous ethanol (EtOH, 20 mL) in a 50 mL two-neck flask equipped with a magnetic stir bar. Hydrazine monohydrate (7 mL, excess) was added dropwise to the reaction mixture under a nitrogen atmosphere at room temperature. The reaction mixture was refluxed for 16 h, followed by cooling in an ice bath. The byproduct phthaloylhydrazide was removed, as a white precipitate, by filtration of the reaction mixture. This precipitate was washed with diethyl ether. The combined filtrates were concentrated under reduced pressure to induce further precipitation of the byproduct. This cycle of filtration and concentration to remove the byproduct was repeated five times until no byproduct was observed by TLC (ethyl acetate (EA)/hexanes (HX): 1/1) under UV. After the last filtration to remove the byproduct, the filtrate was concentrated to a yellow oil. This oil was dissolved into 4 N HCl in dioxane (20 mL) in a 50 mL round-bottom flask equipped with a magnetic stir bar. The reaction was stirred at room temperature for 2 h. The reaction mixture was concentrated under reduced pressure to a yellow oil, which was mixed with Et₂O to give a white solid. The solid was collected by filtration and washed with Et₂O (30 mL) to afford 5 as the HCl salt (1.5 g, 54%): ¹H NMR (400 MHz, DMSO-d₆) δ ppm 9.0 (br s, 3H, NH₂ and HCl), 8.26 (1H, dd, J=8.3 Hz, 2.0 Hz), 8.14 (1H, d, J=8.3 Hz), 8.10 (1H, d, J=2.0 Hz), 4.27 (2H, s); ¹³C NMR (101 MHz, DMSO-d₆) δ ppm 137.6, 132.3, 130.3 (q, ³J_{CF}=2.2 Hz), 129.8 (q, ²J_{CF}=33 Hz), 128.5 (q, ²J_{CF}=31 Hz), 123.7 (q, ¹J_{CF}=274 Hz), 123.7 (q, ¹J_{CF}=274 Hz), 123.4 (dq, ³J_{CF}=2.2 Hz), 38.4; ¹⁹F NMR (376 MHz, DMSO-d₆) δ ppm −58.8, −61.3; MS (m/z) [M+H]⁺, calcd for C₉H₈F₆N, 244.0561, found 244.0574.

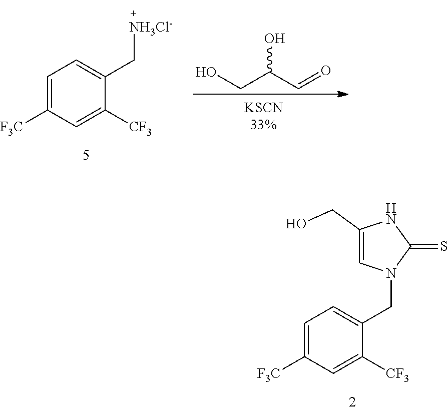

4-(Hydroxymethyl)-1-[[2,4-bis(trifluoromethyl)phenyl]methyl]-2H-imidazole-2-thione (2). A solution of glyceraldehyde in acetonitrile (0.33 mL, 1 μg·mL⁻¹, 3.6 mmol, 1 equiv) was added to a suspension of 5 (1 g, 3.6 mmol, 1 equiv) in acetonitrile (3.6 mL) in a 25 mL two-neck round-bottom flask equipped with a magnetic stir bar. After addition of potassium isothiocyanate (0.5 g, 5.1 mmol, 1.4 equiv) and glacial acetic acid (0.42 mL, 7.2 mmol, 2.2 equiv) the resulting suspension was heated to 60° C. for 16 h. The reaction mixture was cooled to room temperature and diluted with 30 mL of EtOAc to form a brown-yellow solution, which was decolorized by activated charcoal for 30 min. The charcoal was removed by filtration. The filtrate was concentrated to an oil, which was purified by silica-gel chromatography (gradient elution from 100:0 DCM/MeOH to 92:8 DCM/MeOH over 40 min). Fractions with the desired product (R_{f}=0.4 in 95:5 DCM/MeOH) were concentrated to dryness to afford compound 2 (422 mg, 33% yield) as a white solid: ¹H NMR (400 MHz, DMSO-d₆) δ ppm 12.5 (br s, NH), 8.10, (1H, dd, J=8.3 Hz, 2.0 Hz), 8.01 (1H, s), 7.10 (1H, d, J=8.3 Hz), 7.05 (1H, d, J=2.0 Hz), 5.53 (2H, s), 5.25 (br s, OH), 4.0 (2H, s); ¹³C NMR (101 MHz, DMSO-d₆) δ ppm 162.8, 141.0, 130.3 (q, ³J_{CF}=2.2 Hz), 130.0, 129.4, 128.9 (q, ²J_{CF}=33 Hz), 127.8 (q, ²J_{CF}=31 Hz), 123.9 (q, ¹J_{CF}=274 Hz), 123.8 (q, ¹J_{CF}=274 Hz), 123.6 (dq, ³J_{CF}=2.2 Hz), 124.1 (q, ¹J_{CF}=274 Hz), 123.8 (dq, ³J_{CF}=2.2 Hz), 116.5, 54.1, 46.1; MS (m/z): [M+H]⁺, calcd for C₁₃H₁₁F₆N₂OS, 357.0496, found 357.0489.

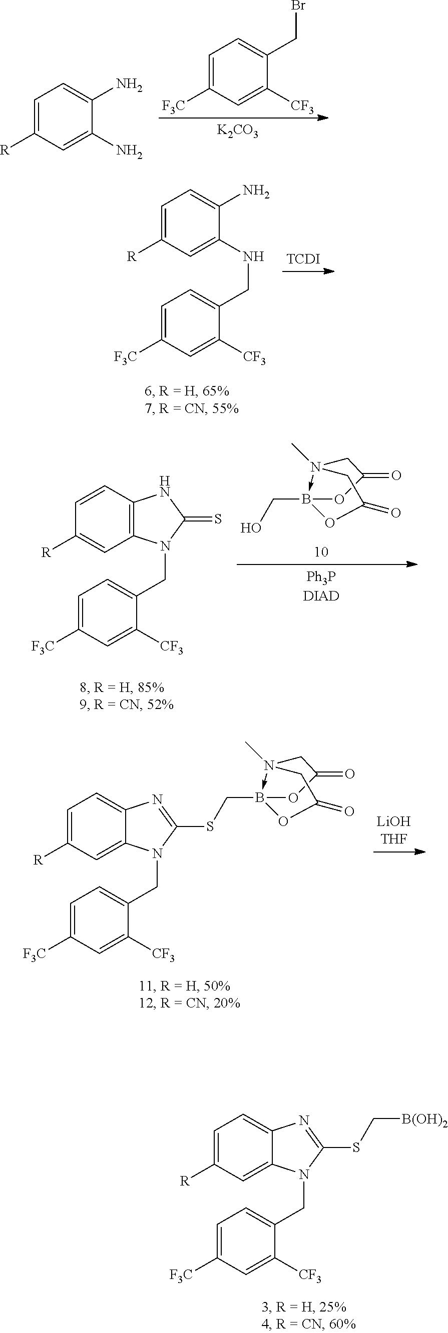

General synthesis for boronic acids 3 and 4. Compound 10 was synthesized by a reported procedure (Chem. Commun. 51, 3608-11 (2015)).

General procedure for mono N-alkylation of o-phenylenediamine derivatives with 2,4-bis(trifluoromethyl)benzyl bromide. In a 100 mL three-neck round-bottom flask equipped with a magnetic stir bar, o-phenylenediamine (45.0 mmol, 4.5 equiv) and potassium carbonate (K₂CO₃) (3.75 g, 20.0 mmol, 2.0 equiv) were added, followed by the addition of 40 mL of anhydrous DMF to give a red-colored suspension. A portion of 2,4-bis(trifluoromethyl)benzylbromide (1.90 mL, 3.10 g, 10.0 mmol, 1.0 equiv) was added dropwise over 1 h. The mixture was stirred at room temperature under an atmosphere of nitrogen for 24 h. The reaction mixture was diluted with EtOAc (0.1 L), and washed with water (3×0.2 L) and brine (0.2 L). The organic layer was dried (Na₂SO₄) and filtered. The filtrate was evaporated to give a red oil. This oil was purified by silica chromatography (gradient elution from 100:0 hexanes/EtOAc to 20:80 hexanes/EtOAc over 60 min) to afford the desired mono N-alkylation products.

N¹-[[2,4-Bis(trifluoromethyl)phenyl]methyl]-1,2-benzenediamine (6). Obtained as a colorless amorphous solid (2.17 g, 65%): ¹H NMR (400 MHz, DMSO-d₆) δ ppm 8.04 (2H, m), 7.82 (1H, d, J=8.1 Hz), 6.62 (1H, dd, J=7.6, 1.7), 6.41 (2H, ddd, J=16.2, 7.5, 1.6 Hz), 6.07 (1H, dd, J=7.7, 1.5), 5.49 (1H, t, J=5.8), 4.65 (2H, s), 4.56 (2H, d, J=5.7); ¹³C NMR (101 MHz, DMSO-d₆) δ ppm 144.7, 136.0, 135.0, 130.1, 128.4 (q, ²J_{CF}=33 Hz), 127.7 (q, ²J_{CF}=31 Hz), 127.2, 124.7 (q, ¹J_{CF}=274 Hz), 123.5 (q, ¹J_{CF}=274 Hz), 123.4 (dq, ³J_{CF}=2.2 Hz), 118.1, 118.0, 115.0, 110.3, 43.7; MS (m/z) [M+H]⁺, calcd for C₁₅H₁₃F₆N₂ 335.0983, found 335.0990.

4-Amino-3-[[[2,4-bis(trifluoromethyl)phenyl]methyl]amino]benzonitrile (7). Obtained as a colorless amorphous solid (1.98 g, 55%): This compound as isolated after column chromatography as a solid containing 15% (¹H-NMR) of its isomer,1-[2,4-bis(trifluoromethyl)benzyl]-4-cyano-1,2-phenylenediamine. Compound 7 was used in the next step without further purification. ¹H NMR (400 MHz, DMSO-d₆) δ ppm 8.07 (2H, dd, J=5.0), 7.78 (1H, d, J=8.1 Hz), 6.91 (1H, dd, J=8.1, 1.8), 6.65 (1H, d, J=8.1), 6.38 (1H, d, J=1.8 Hz), 5.81 (1H, d, J=5.7), 5.74 (2H, s), 4.58 (2H, d, J=5.7); ¹³C NMR (101 MHz, DMSO-d₆) δ ppm 143.6, 141.3, 139.3, 136.2, 134.5, 130.1, 128.7 (q, ²J_{CF}=33 Hz), 128.0 (q, ²J_{CF}=31 Hz), 124.2 (q, ¹J_{CF}=274 Hz), 123.9 (q, ¹J_{CF}=274 Hz), 123.7, 123.4 (dq, 3J_{CF}=2.2 Hz), 121.5, 113.5, 97.5, 43.7; ¹⁹F NMR (376 MHz, DMSO-d₆) δ ppm −59.5, −61.2; MS (m/z): [M+H]⁺, calcd for C₁₆H₁₂F₆N₃ 360.0935, found 360.0950.

General procedure for cyclization of N-alkyl phenylenediamines 6 and 7 with TCDI to benzimidazole-2-thione 8 and 9. In a 50 mL round-bottom flask equipped with a magnetic stir bar, N-alkylated phenylenediamine 6 or 7 (7.00 mmol, 1.0 equiv) and 1,1′-thiocarbonyldiimidazole (10.5 mmol, 1.5 equiv) were dissolved in anhydrous DMF (10.0 mL). The reaction mixture was refluxed for 3 h and then cooled to room temperature. The reaction mixture was poured into a beaker containing 100 mL of ice water and stirred for 30 min to give an off-white precipitate. The precipitate was filtered and washed with deionized water (50 mL) and n-pentane (50 mL). The solid product was collected and dried overnight in vacuo.

1-[[2,4-Bis(trifluoromethyl)phenyl]methyl]benzimidazole-2-thione (8). Obtained as a colorless amorphous solid (2.23 g, 85%), which was used without further purification: ¹H NMR (400 MHz, DMSO-d₆) δ ppm 13.15 (1H, brs), 8.14 (1H, d, J=2.0), 7.96 (1H, dd, J=8.3, 2.0), 7.30 (1H, m), 7.24 (1H, ddd, J=16.2, 7.5, 1.6 Hz), 7.18 (2H, m), 6.91 (1H, d, J=8.2), 5.76 (2H, s); ¹³C NMR (101 MHz, DMSO-d₆) δ ppm 169.8, 139.9, 132.8, 130.5, 129.00 (q, ²J_{CF}=33 Hz), 129.1, 128.8 (q, ²J_{CF}=31 Hz), 128.5, 124.0, 123.7 (q, ¹J_{CF}=274 Hz), 123.8 (dq, ³J_{CF}=2.2 Hz), 123.5 (q, ¹J_{CF}=274 Hz), 123.2, 110.6, 110.0, 43.7; ¹⁹F NMR (376 MHz, DMSO-d₆) δ ppm −59.6, −61.4; MS (m/z): [M+H]⁺, calcd for C₁₆H₁₁F₆N₂S 377.0547, found 377.0555.

1-[2,4-bis(trifluoromethyl)benzyl]-5-nitrile-benzimidazole-2-thione (9). The crude product was purified by silica-gel chromatography (gradient elution from 100:0 hexanes/EtOAc to 75:25 HX/EtOAc over 75 min). Fractions containing the product were concentrated and dried in vacuo overnight to obtain the title compound as a white solid (1.55 g, 55%): ¹H NMR (400 MHz, DMSO-d₆) δ ppm 13.58 (1H, s), 8.14 (1H, d, J=2.0), 7.92 (2H, d, J=1.6), 7.68 (1H, d, J=1.6), 7.43 (1H, d, J=8.3), 6.89 (1H, d, J=8.3), 5.76 (2H, s); ¹³C NMR (101 MHz, DMSO-d₆) δ ppm 171.8, 139.5, 134.9, 133.4, 130.4, 128.9 (q, ²J_{CF}=33 Hz), 128.4, 128.3, 127.8 (q, ²J_{CF}=31 Hz), 123.7 (q, ¹J_{CF}=274 Hz), 123.8 (dq, ³J_{CF}=2.2 Hz), 123.5 (q, ¹J_{CF}=274 Hz), 119.6, 114.1, 111.3, 105.1, 44.2; ¹⁹F NMR (376 MHz, DMSO-d₆) δ ppm −58.6, −61.1; MS (m/z): [M+H]⁺, calcd for C₁₇H₁₀F₆N₃S 402.0500, found 402.0504.

General procedure for syntheses of MIDA boronate esters 11 and 12. Diisopropyl azodicarboxylate (64.9 mg, 0.321 mmol) was added slowly to an anhydrous THF solution (4.5 mL) of 10 (50.0 mg, 0.27 mmol), polymer-bound triphenylphosphine PPh₃ (0.40 mmol, 0.25 g), and benzimidazole (0.40 mmol) in a two-dram vial equipped with a magnetic stir bar at room temperature. The reaction mixture was stirred for 6 h. The triphenylphosphine resin was filtered and washed with EtOAc (5 mL×6). The filtrate was concentrated under reduced pressure to a yellow oil. The oil was suspended in t-BuOMe (10 mL), with the appearance of a white precipitate. The product precipitate was collected by filtration, and the filtrate was discarded. The product precipitate was resuspended in t-BuOMe. This cycle of resuspension and decantation was repeated three times. The crude product was purified by silica chromatography (0-5% MeOH/DCM over 30 min) to afford the MIDA-boronate ester as a white amorphous solid.

(T-4)-[N-[(Carboxy-κO)methyl]-N-methylglycinato(2-)-κN,κO][[N¹-[[2,4-bis(trifluoromethyl)phenyl]methyl]-1H-benzimidazole-1-yl]thiomethyl]boron (11). Obtained as a colorless amorphous solid (73 mg, 50%): ¹H NMR (400 MHz, CH₃CN-d₃) δ ppm 8.52 (2H, m), 8.15 (1H, s), 7.82 (1H, dd, J=8.3, 2.0), 7.46 (1H, d, J=8.0), 7.33 (1H, ddd, J=8.2, 6.2, 2.0 Hz), 7.24 (2H, m), 6.77 (1H, d, J=8.2), 5.82 (2H, s), 4.22 (2H, s), 4.11 (2H, s), 4.03 (2H, s), 3.34 (3H, s); ¹³C NMR (101 MHz, CH₃CN-d₃) δ ppm 169.4, 169.0, 139.8, 139.8, 133.0, 131.7, 130.3, 128.5 (q, ²J_{CF}=33 Hz), 128.8 (q, ²J_{CF}=31 Hz), 128.5, 123.7, 123.5, 123.5 (q, ¹J_{CF}=274 Hz), 123.4 (dq, ³J_{CF}=2.2 Hz), 123.3 (q, ¹J_{CF}=274 Hz), 111.3, 109.9, 62.5, 51.8, 46.9, 44.6, 21.8; ¹⁹F NMR (376 MHz, DMSO-d₆) δ ppm −59.8, −61.4; MS (m/z): [M+H]⁺, calcd for C₂₂H₁₉BF₆N₃O₄S 546.1094, found 546.1090.

(T-4)-[N-[(Carboxy-κO)methyl]-N-methylglycinato(2-)-κN,κO][[N¹-[[6-cyano-2,4-bis(trifluoromethyl)phenyl]methyl]-1H-benzimidazole-1-yl]thiomethyl]boron (12). Obtained as a colorless amorphous solid (38 mg, 25%): ¹H NMR (400 MHz, CH₃CN-d₃) δ ppm 8.12 (1H, s), 7.71 (2H, m), 7.82 (2H, m), 7.58 (1H, d, J=8.4 Hz), 7.51 (1H, d, J=1.6 Hz) 6.73 (1H, d, J=8.2 Hz), 5.83 (2H, s), 4.13 (2H, s), 4.04 (4H), 4.05 (2H, s), 3.21 (3H, s); ¹³C NMR (101 MHz, CH₃CN-d₃) 6 ppm 172.8, 168.9, 168.2, 139.5, 138.9, 136.6, 132.2, 130.2, 128.7 (q, ²J_{CF}=35 Hz), 128.3, 128.2, 127.2 (q, ²J_{CF}=35 Hz), 124.3 (q, ¹J_{CF}=274 Hz), 124.3 (dq, ³J_{CF}=2.2 Hz), 124.1 (q, ¹J_{CF}=274 Hz), 119.3, 113.6, 112.0, 106.3, 62.8, 46.9, 44.6, 21.8; ¹⁹F NMR (376 MHz, DMSO-d₆) δ ppm −58.8, −61.2; MS (m/z): [M+H]⁺, calcd for C₂₃H₁₈BF₆N₃O₄S 571.1046, found 571.1050.

General procedure for deprotection of boronate esters 11 and 12 to boronic acids 3 and 4. Boronate ester (0.21 mmol) and lithium hydroxide monohydrate (34 mg, 0.84 mmol, 4 equiv.) were dissolved into THF/H₂O (95:5 v/v, 5 mL) in a 25 mL flask equipped with a magnetic stir bar at room temperature. The reaction was stirred for 6 h and followed by TLC until starting material was consumed. The reaction mixture was then concentrated under reduced pressure to a colorless oil. The colorless oil was dissolved into t-BuOMe (10 mL) and washed with sat. NaHCO₃ solution (5 mL). The aqueous layer was washed with t-BuOMe (10 mL×3). The organic layer was collected and dried over Na₂SO₄. The desired boronic acids were obtained as white amorphous solid by concentrating the organic layer and dried in vacuo overnight.

(T-4)-[N-[(Carboxy-κO)methyl]-N-methylglycinato(2-)-κN,κO][[N′-[[2,4-bis(trifluoromethyl)phenyl]methyl]-1H-benzimidazole-1-yl]thiomethyl]boronic acid (3). Compound 3 is unstable on TLC, obtained as colorless amorphous solid as a 1:1 complex of 3:MTBE by NMR, (23 mg, 25%), 95% purity by LC-MS: ¹H NMR (400 MHz, DMSO-d₆) δ ppm 8.50 (2H, brs), 8.13 (1H, s), 7.75 (1H, dd, J=8.3, 2.0), 7.30 (1H, d, J=8.0), 7.26 (1H, ddd, J=8.2, 6.2, 2.0 Hz), 7.20 (2H, m), 6.6 (1H, d, J=8.2), 5.81 (2H, s), 3.9 (2H, s); ¹³C NMR (101 MHz, DMSO-d₆) δ ppm 162.0, 139.8, 139.8, 133.0, 131.7, 130.3, 128.5 (q, ²J_{CF}=33 Hz), 128.8 (q, ²J_{CF}=31 Hz), 128.5, 123.7, 123.5, 123.5 (q, ¹J_{CF}=274 Hz), 123.4 (dq, ³J_{CF}=2.2 Hz), 123.3 (q, ¹J_{CF}=274 Hz), 109.9, 51.8, 44.6. MS (m/z): [M+H]⁺, calcd for C₁₇H₁₃BF₆N₂O₂S 435.0773, found 435.0636.

(T-4)-[N-[(Carboxy-κO)methyl]-N-methylglycinato(2-)-κN,κO][[N]-[[2,4-bis(trifluoromethyl)phenyl]methyl]-1H-benzimidazole-1-yl]thiomethyl]boronic acid (4). Compound 4 is unstable on TLC, obtained as a colorless amorphous solid as a 1:1 complex of 4:MTBE (58 mg, 60%), 95% purity by LC-MS. ¹H NMR (400 MHz, DMSO-d₆) δ ppm 8.13 (2H, brs), 7.97 (1H, s), 7.91 (1H, dd, J=8.3, 2.0), 7.74 (1H, d, J=8.0), 7.66 (1H, ddd, J=8.2, 6.2, 2.0 Hz), 6.92 (2H, m), 5.80 (2H, s), 3.88 (2H, s); ¹³C NMR (101 MHz, DMSO-d₆) δ ppm 165.9, 139.5, 138.9, 136.6, 132.2, 130.2, 128.7 (q, ²J_{CF}=35 Hz), 128.3, 128.2, 127.2 (q, ²J_{CF}=35 Hz), 124.3 (q, ¹J_{CF}=274 Hz), 124.3 (dq, 3J_{CF}=2.2 Hz), 124.1 (q, ¹J_{CF}=274 Hz), 119.3, 113.6, 112.0, 106.3, 62.8, 46.9, 44.6, 21.8; ¹⁹F NMR (376 MHz, DMSO-d₆) δ ppm −58.8, −61.2; MS (m/z): [M+H]⁺, calcd for C₁₈H₁₂BF₆N₂O₂S 460.0647, found 460.0745.

### Example 3. General Synthetic Schemes for Inhibitors, and Compound Data

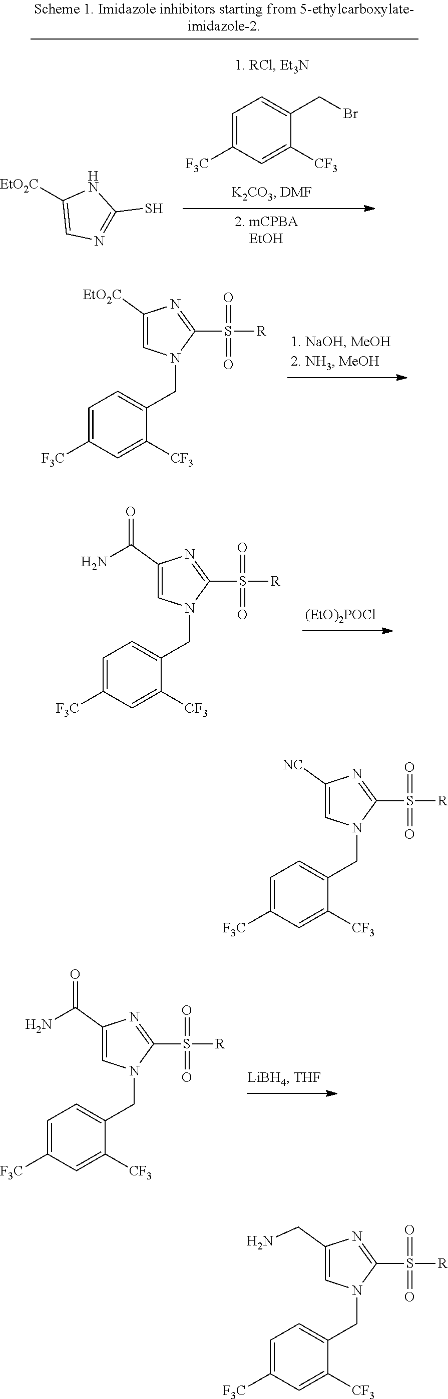

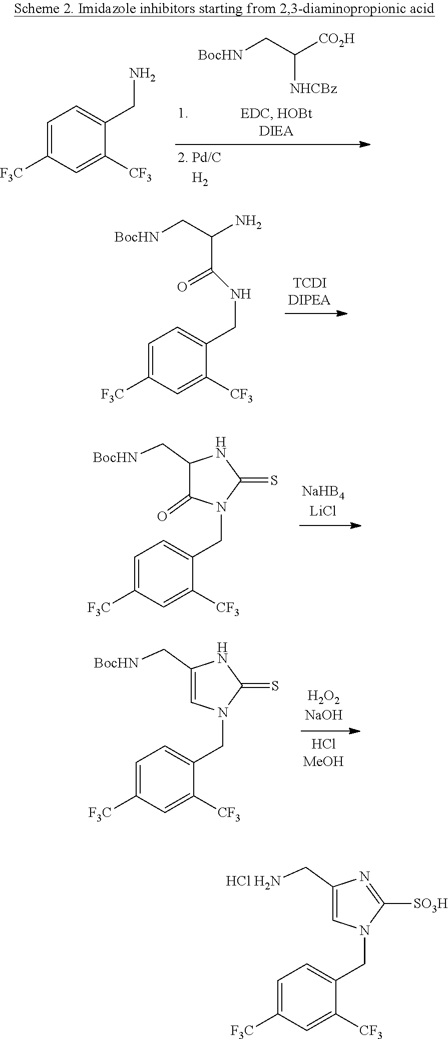

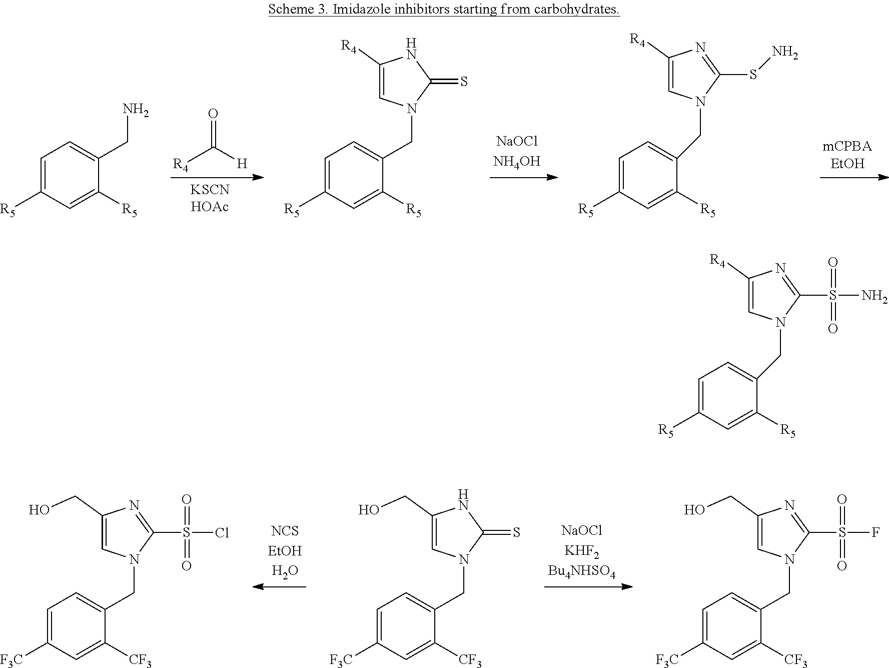

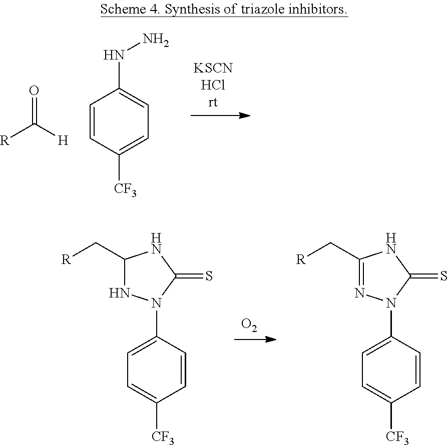

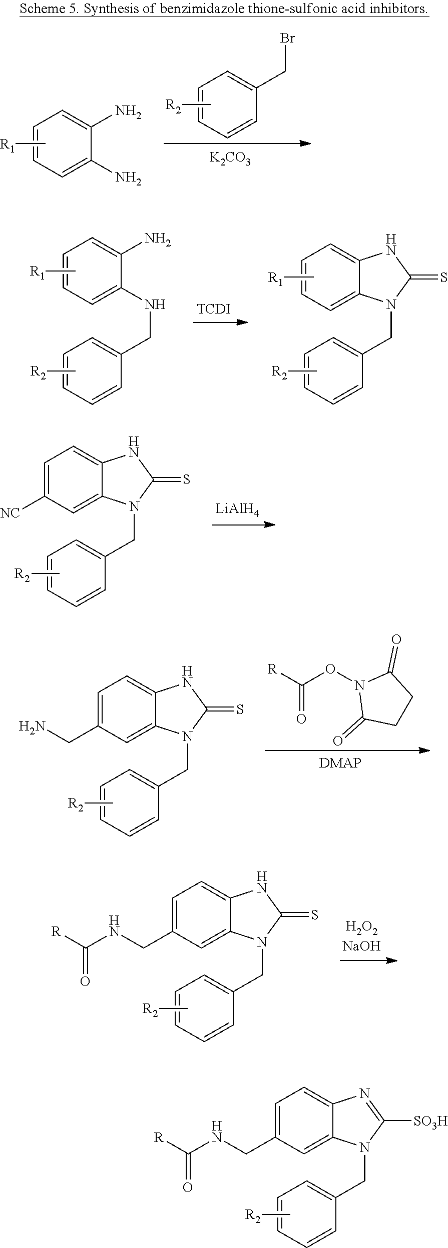

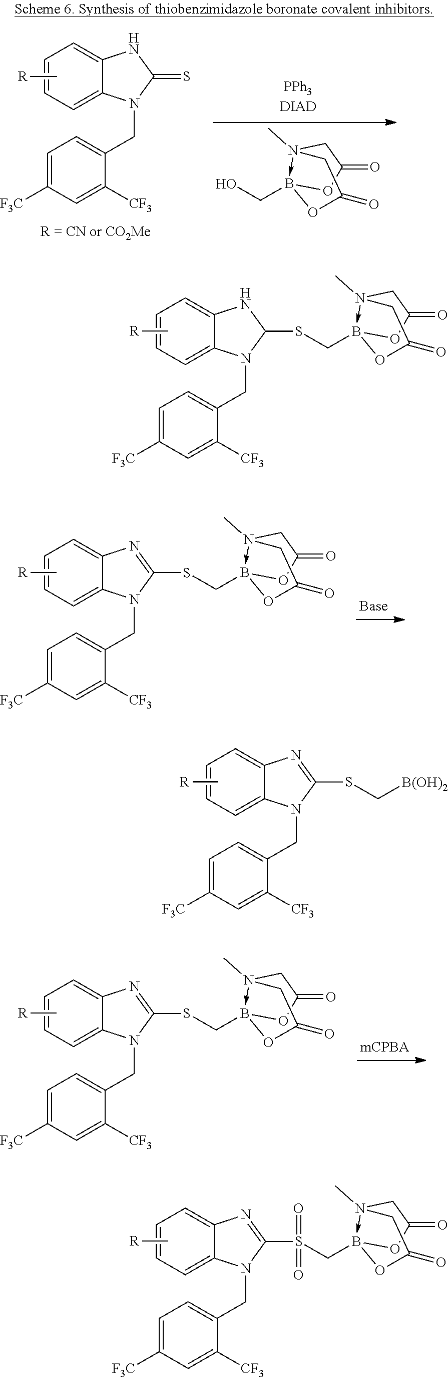

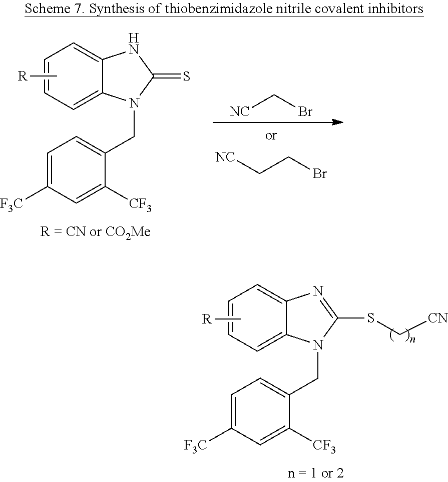

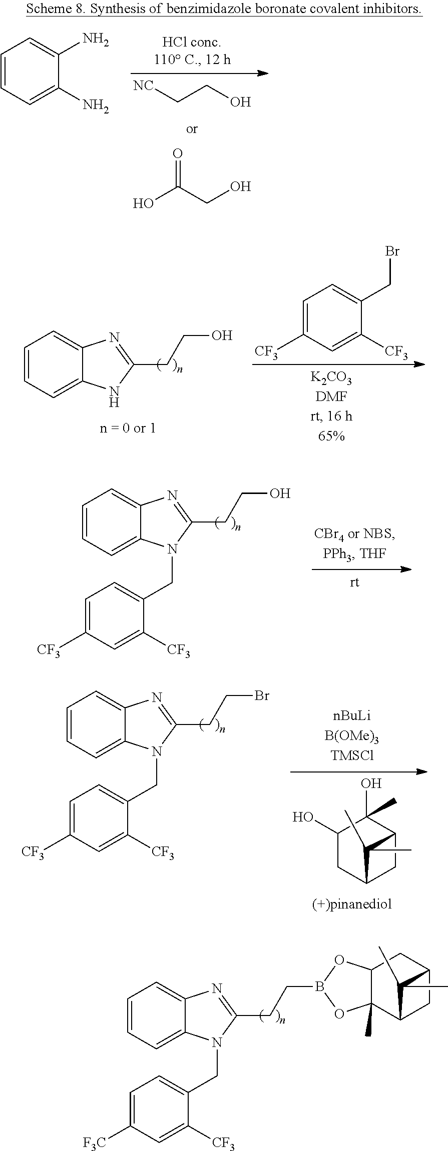

Benzimidazole Inhibitors (% Inhibition at 100 mg/L⁻¹ shown for BlaR, bacterial growth).

| | |
|---|---|
| 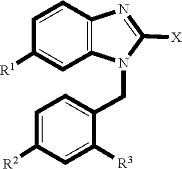 8, R¹ = H, R₂ = R₃ = CF₃, X = SH, 65; 10 11, R¹ = H, R₂ = F, R₃ = CF₃, X = SO₃H, 78; 0 12, R¹ = H, R₂ = Cl, R₃ = H, X = SO₃H, 72; 0 13, R¹ = H, R₂ = H, R₃ = CF₃, X = SO₃H, 69; 0 R² = R³ = CF₃ | 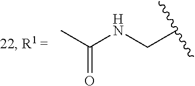 [SMILES: *CNC(C)=O] X = SH, 70; 0 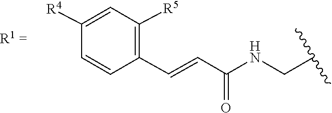 |
| 1, R¹ = H, X = SO₃H, 82; 0 9, R¹ = CN, X = SH, 100; 97 | R² = R³ = CF₃ 23, R⁴ = F, R⁵ = H, X = SH, 76; 70 |
| 14, R¹ = CH₂NH₂, X = SH, 60; 0 | 24, R⁴ = F, R⁵ = H, X = SO₃H, 100; 100 |
| 15, R¹ = CO₂Me, X = SH, 10; 0 | 25, R⁴ = CI, R⁵ = Cl, X = SH, 75; 50 |
| 16, R¹ = CO₂Me, X = SO₃H, 20; 0 | 26, R⁴ = F, R⁵ = H, X = SO₃H, 99; 100 |
| 17, R¹ = CH₂OH, X = SH, 0; 99 | 27, R⁴ = CF₃, R⁵ = F, X = SH, 79; 55 |
| 18, R¹ = CH₂OH, X = SO₃H, 97; 0 | 28, R⁴ = CF 3, R⁵ = F, X = SO₃H, 92; 100 |
| 19, R¹ = CN, X = NH₂, 55; 0 | |
| 20, R¹ = CO₂Me, X = NH₂, 100; 0 21, R¹ = CH₂OH, X = NH₂, 89; 0 R² = R³ = CF₃ 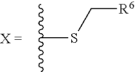 | 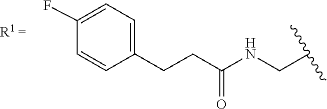 [SMILES: *CNC(=O)CCc1ccc(F)cc1] R² = R³ = CF₃ 29, X = SH, 59; 0 |
| 3, R1 = H, R6 = B(OH)2, 98; 20 | 30, X = SO₃H, 86; 0 |
| 4, R1 = CN, R6 = B(OH)2, 98; 25 | R¹ = H |
| 31, R¹ = H, R⁶ = B(MIDA), 100, 13 | R² = R³ = CF₃ |
| 32, R¹ = CN, R⁶ = B(MIDA), 93, 20 33, R¹ = CO₂Me, R⁶ = B(OH)₂, 95; 13 34, R¹ = CO₂H, R⁶ = B(OH)₂, 92; 18 35, R¹ = CO₂Me, R⁶ = B(MIDA), 98, 33 36, R¹ = CN, R⁶ = CH₂CN, 90; 40 37, R¹ = CN, R⁶ = CH₂CH₂CN, 92, 25 38, R¹ = CO₂Me, R⁶ = CH₂CN, 89; 16 | 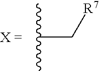 |
| 39, R¹ = CO₂Me, R⁶ = CH₂CH₂CN, 92, 25 | 40, R⁷ = OH, 67; 25 |
| | 41, R⁷ = Br, 78; 36 |
| 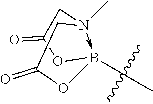 [SMILES: *[B-]12OC(=O)C[N+]1(C)CC(=O)O2] | 42, R⁷ = B(OH)₂. 99; 82 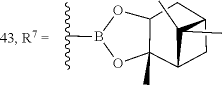 [SMILES: *B1OC2C[C@@H]3C[C@@H](C3(C)C)[C@]2(C)O1] B((+)pinanediol) 99; 84 44, R⁷ = CH₂OH, 100; 40 45, R⁷ = CH₂B((+)pinanediol) 0; 30 46, R⁷ = CH₂B(OH)₂. 0, 49 |

Imidazole and Triazole Inhibitors (% Inhibition at 100 mg/L⁻¹ shown for BlaR, bacterial growth).

| | | |
|---|---|---|
| 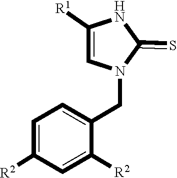 2, R¹ = CH₂OH, R² = CF 3, 92; 0 47, R¹ = CH₂NHBoc, R² = CF₃, 100; 0 48, R¹ = CH₂NH₂, R² = CF₃, 50; 90 | 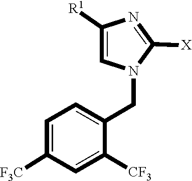 54, R¹ = H, X = SO₃H, 18; 0 R¹ = CN 55, X = H, 0; 0 | R¹ = CH₂NHR 63, R = H, X = SO₃H, 50;18 64, R = Boc, X = SO₃H, 75; 40 65, R = Cbz, 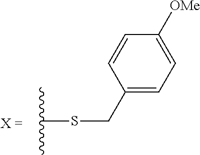 [SMILES: *SCc1ccc(OC)cc1] 19; 0 |
| 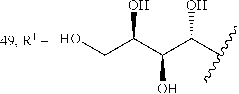 [SMILES: *[C@@H](O)[C@@H](O)[C@H](O)CO] R² = CF₃ 7;0 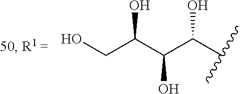 [SMILES: *[C@@H](O)[C@@H](O)[C@H](O)CO] R² = H 13;0 | 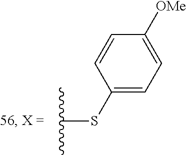 [SMILES: *Sc1ccc(OC)cc1] 57; 0 R¹ = CH₂OH 57, X = SO₂Cl, 83; 0 58, X = Cl, 43; 0 59, X = SO₂F, 100; 83 60, X = SO₃H, 0; 0 | 21, R = H, 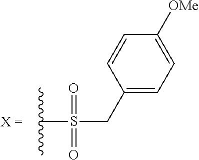 [SMILES: *S(=O)(=O)Cc1ccc(OC)cc1] 100; 0 |
| 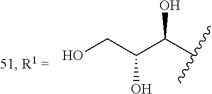 [SMILES: *[C@H](O)[C@H](O)CO] R² = H 11;0 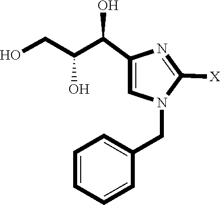 53, X = SO₂NH₂, 100; 49 52, X = SNH₂, 33; 0 R¹ = CO₂Et 66, X = SO₃H, 88; 0 | 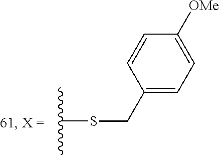 [SMILES: *SCc1ccc(OC)cc1] 23; 0 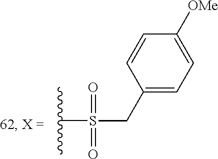 [SMILES: *S(=O)(=O)Cc1ccc(OC)cc1] 75; 0 R¹ = CO₂H 72, X = SO₃H, 11; 0 | 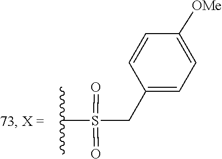 [SMILES: *S(=O)(=O)Cc1ccc(OC)cc1] 77, X = NH, 71; 30 78, X = N, 70; 56 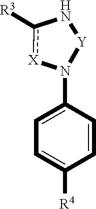 |
| 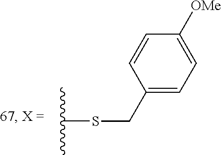 [SMILES: *SCc1ccc(OC)cc1] | 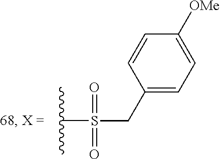 [SMILES: *S(=O)(=O)Cc1ccc(OC)cc1] | 79, R³ = CH₂OH, R⁴ = CF₃, X = NH, Y = CS, 75; 65 80, R³ = B(OH)₂, R⁴ = H X = CH, Y = N, 8; 0 81, R³ = B(MIDA) R⁴ = H X = CH, Y = N, 50; 11 |
| 75; 0 | 62; 0 | |
| 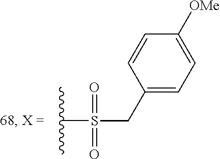 [SMILES: *S(=O)(=O)Cc1ccc(OC)cc1] 70; 0 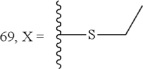 [SMILES: *SCC] 18; 0 | 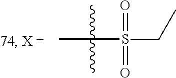 [SMILES: *S(=O)(=O)CC] 61; 18 R¹ = CONH₂ 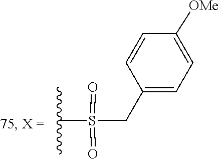 [SMILES: *S(=O)(=O)Cc1ccc(OC)cc1] 83; 0 | |
| 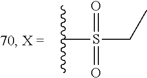 [SMILES: *S(=O)(=O)CC] 62; 0 | 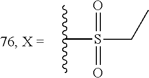 [SMILES: *S(=O)(=O)CC] | |
| 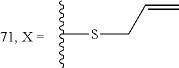 [SMILES: *SCC=C] | 59; 0 | |
| 18; 0 | | |

### Example 4. Pharmaceutical Dosage Forms

The following formulations illustrate representative pharmaceutical dosage forms that may be used for the therapeutic or prophylactic administration of a compound of a formula described herein, a compound specifically disclosed herein, or a pharmaceutically acceptable salt or solvate thereof (hereinafter referred to as ‘Compound X’):

| (i) Tablet 1 | mg/tablet |
|---|---|
| ‘Compound X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

TABLE t

| (ii) Tablet 2 | mg/tablet |
|---|---|
| ‘Compound X’ | 20.0 |
| Microcrystalline cellulose | 410.0 |
| Starch | 50.0 |
| Sodium starch glycolate | 15.0 |
| Magnesium stearate | 5.0 |
| | 500.0 |
| (iii) Capsule | mg/capsule |
| ‘Compound X’ | 10.0 |
| Colloidal silicon dioxide | 1.5 |
| Lactose | 465.5 |
| Pregelatinized starch | 120.0 |
| Magnesium stearate | 3.0 |
| | 600.0 |
| (iv) Injection 1 (1 mg/mL) | mg/mL |
| ‘Compound X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |
| (v) Injection 2 (10 mg/mL) | mg/mL |
| ‘Compound X’ (free acid form) | 10.0 |
| Monobasic sodium phosphate | 0.3 |
| Dibasic sodium phosphate | 1.1 |
| Polyethylene glycol 400 | 200.0 |
| 0.1 N Sodium hydroxide solution | q.s. |
| (pH adjustment to 7.0-7.5) | |
| Water for injection | q.s. ad 1 mL |
| (vi) Aerosol | mg/can |
| ‘Compound X’ | 20 |
| Oleic acid | 10 |
| Trichloromonofluoromethane | 5,000 |
| Dichlorodifluoromethane | 10,000 |
| Dichlorotetrafluoroethane | 5,000 |
| (vii) Topical Gel 1 | wt. % |
| ‘Compound X’ | 5% |
| Carbomer 934 | 1.25% |
| Triethanolamine | q.s. |
| (pH adjustment to 5-7) | |
| Methyl paraben | 0.2% |
| Purified water | q.s. to 100 g |
| (viii) Topical Gel 2 | wt. % |
| ‘Compound X’ | 5% |
| Methylcellulose | 2% |
| Methyl paraben | 0.2% |
| Propyl paraben | 0.02% |
| Purified water | q.s. to 100 g |
| (ix) Topical Ointment | wt. % |
| ‘Compound X’ | 5% |
| Propylene glycol | 1% |
| Anhydrous ointment base | 40% |
| Polysorbate 80 | 2% |
| Methyl paraben | 0.2% |
| Purified water | q.s. to 100 g |
| (x) Topical Cream 1 | wt. % |
| ‘Compound X’ | 5% |
| White bees wax | 10% |
| Liquid paraffin | 30% |
| Benzyl alcohol | 5% |
| Purified water | q.s. to 100 g |
| (xi) Topical Cream 2 | wt. % |
| ‘Compound X’ | 5% |
| Stearic acid | 10% |
| Glyceryl monostearate | 3% |
| Polyoxyethylene stearyl ether | 3% |
| Sorbitol | 5% |
| Isopropyl palmitate | 2 % |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Compound X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

1. A compound of formula I: 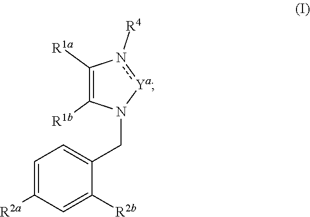
wherein, [symbol]
is a double bond or a single bond;
Y^{a} is 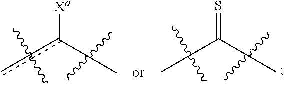
wherein X^{a} is SG³, H, halo, N(G¹)₂, SO₂G², or —CH₂R⁶;
G¹ is each independently H, —(C₁-C₆)alkyl, Boc, or Cbz;
G² is halo, OH, N(G¹)₂, —(C₁-C₆)alkyl, or —CH₂Ar;
G³ is —CH₂R⁷, H, —(C₁-C₆)alkyl, —(C₁-C₆)alkenyl, N(G¹)₂, or Ar;
G⁴ is OH, N(G¹)₂, or —O(C₁-C₆)alkyl;
Ar is a phenyl substituted with —O(C₁-C₆)alkyl;
R^{1a} and R^{1b} taken together with the carbon atoms to which they are attached in formula I form a benzo ring substituted with R^{1c}; or
R^{1a} is H, CN, —C(═O)G⁴, —CH₂N(G¹)₂, or —(C₁-C₆)alkyl substituted with one or more OR^{2c}; and
R^{1b} is H, halo, or —(C₁-C₆)alkyl;
R^{1c} is CN, H, —C(═O)G⁴, —CH₂OR^{2c}, —CH₂N(G¹)₂, —CH₂N(G¹)COCH₃, or —CH₂N(G¹)CO(Y^{b})Ph, wherein Ph is substituted with R^{5a} and/or R^{5b}, and Y^{b} is —CH═CH— or —CH₂CH₂—;
R^{2a} and R^{2b} are each independently CF₃, H, halo, SF₅, or —(C₁-C₆)alkyl optionally substituted with one or more halo;
R^{2c} is each independently H or —(C₁-C₆)alkyl;
R⁴ is a lone pair when Y^{a} is 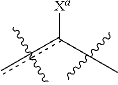
and [symbol] is a double bond; or
R⁴ is H or —(C₁-C₆)alkyl when Y^{a} is 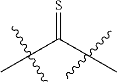 [SMILES: *C(*)=S]
and [symbol] is a single bond;
R^{5a} and R^{5b} are each independently H, halo, CF₃, SF₅, or —(C₁-C₆)alkyl optionally substituted with one or more halo;
R⁶ is halo, OR^{2c}, —CH₂OR^{2c}, B(OR^{2c})₂, —CH₂B(OR^{2c})₂, B(pinanediol), —CH₂B(pinanediol), or 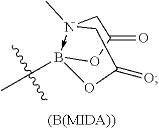 [SMILES: *[B-]12OC(=O)C[N+]1(C)CC(=O)O2]
and
R⁷ is B(OR^{2c})₂, B(MIDA), Ar, or —(CH₂)ₙCN wherein n is 1 to 6,
or a pharmaceutically acceptable salt thereof.

2. The compound of claim 1, wherein X^{a} is SCH₂R⁷, H, Cl, NH₂, SO₃H, SO₂F, SO₂Cl, SO₂NH₂, SO₂Et, SO₂CH₂Ar, SH, SNH₂, SEt, S(allyl), SAr, SCH₂Ar, or CH₂R⁶, wherein Ar is an anisole moiety.

3. The compound of claim 1, wherein Ar is para-anisole.

4. The compound of claim 1, wherein R^{1a} is H, CN, CO₂H, CONH₂, CO₂Et, CH₂NH₂, CH₂NHBoc, CH₂NHCbz, CH₂OH, CH(OH)CH(OH)CH₂OH, or CH(OH)CH(OH)CH(OH)CH₂OH.

5. The compound of claim 1, wherein R^{1a} is

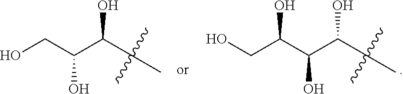 [SMILES: *[C@H](O)[C@H](O)CO ; *[C@@H](O)[C@@H](O)[C@H](O)CO]

6. The compound of claim 1, wherein R^{2a} and R^{2b} are each CF₃.

7. The compound of claim 1, wherein R^{1c} is CN, H, CO₂H, CO₂Me, CH₂OH, CH₂NH₂, CH₂NHCOCH₃, CH₂NHCOCH₂CH₂Ph, or CH₂NHCOCH═CHPh, wherein Ph is substituted with R^{5a} and R^{5b}.

8. The compound of claim 1, wherein R^{1c} is —CH₂NHCOCH₂CH₂Ph or —CH₂NHCOCH═CHPh wherein the double bond is trans, wherein Ph is

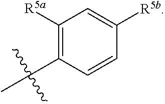

9. The compound of claim 1, wherein R^{5a} and R^{5b} are each independently H, Cl, F, or CF₃.

10. The compound of claim 1, wherein R⁷ is CN, CH₂CN, CH₂CH₂CN, B(OH)₂, or B(MIDA).

11. The compound of claim 1, wherein R⁶ is Br, OH, CH₂OH, B(OH)₂, CH₂B(OH)₂, B((+)pinanediol), CH₂B((+)pinanediol), or B(MIDA).

12. The compound of claim 1, as represented by formula II, III, or IV: 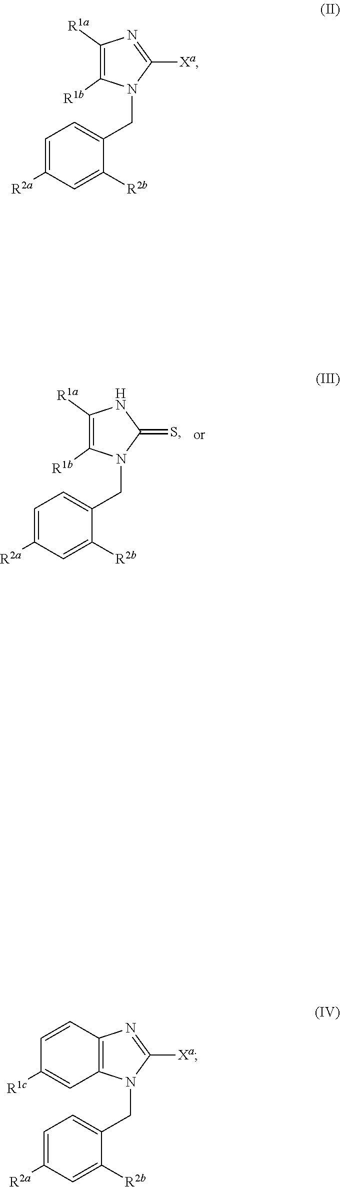
or a pharmaceutically acceptable salt thereof,
wherein for formula I, X^{a} is SG³, H, halo, N(G¹)₂, SO₂G², or —CH₂R⁶.

13. The compound of claim 1, as represented by formula V: 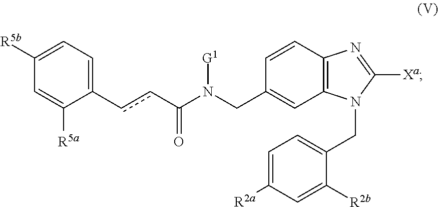
or a pharmaceutically acceptable salt thereof.

14. The compound of claim 1, wherein the compound is compound 4 or 59: 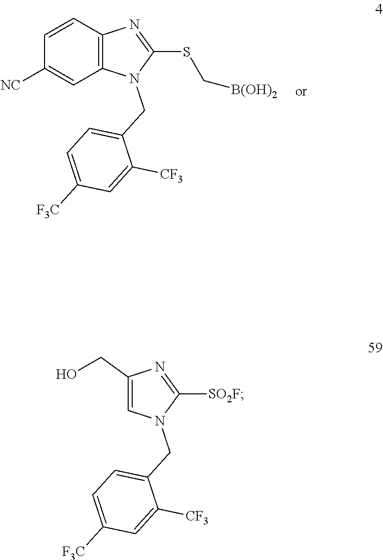 [SMILES: N#Cc1ccc2nc(SCB(O)O)n(Cc3ccc(C(F)(F)F)cc3C(F)(F)F)c2c1 ; O=S(=O)(F)c1nc(CO)cn1Cc1ccc(C(F)(F)F)cc1C(F)(F)F]
or a pharmaceutically acceptable salt thereof.

15. The compound of claim 1, wherein the compound is: 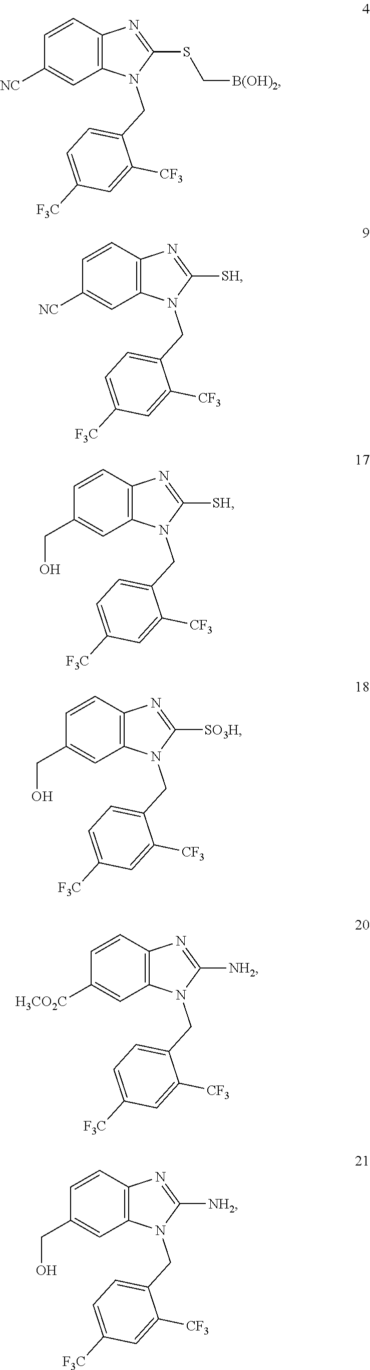 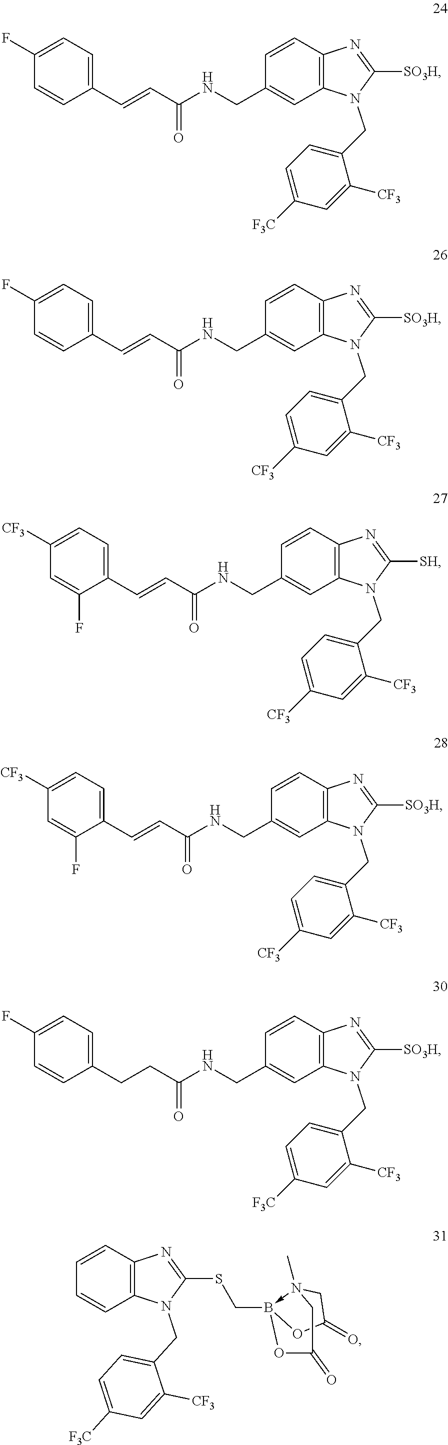 [SMILES: O=C(/C=C/c1ccc(F)cc1)NCc1ccc2nc(S(=O)(=O)O)n(Cc3ccc(C(F)(F)F)cc3C(F)(F)F)c2c1 ; O=C(/C=C/c1ccc(F)cc1)NCc1ccc2nc(S(=O)(=O)O)n(Cc3ccc(C(F)(F)F)cc3C(F)(F)F)c2c1 ; O=C(/C=C/c1ccc(C(F)(F)F)cc1F)NCc1ccc2nc(S)n(Cc3ccc(C(F)(F)F)cc3C(F)(F)F)c2c1 ; O=C(/C=C/c1ccc(C(F)(F)F)cc1F)NCc1ccc2nc(S(=O)(=O)O)n(Cc3ccc(C(F)(F)F)cc3C(F)(F)F)c2c1 ; O=C(CCc1ccc(F)cc1)NCc1ccc2nc(S(=O)(=O)O)n(Cc3ccc(C(F)(F)F)cc3C(F)(F)F)c2c1 ; C[N+]12CC(=O)O[B-]1(CSc1nc3ccccc3n1Cc1ccc(C(F)(F)F)cc1C(F)(F)F)OC(=O)C2] 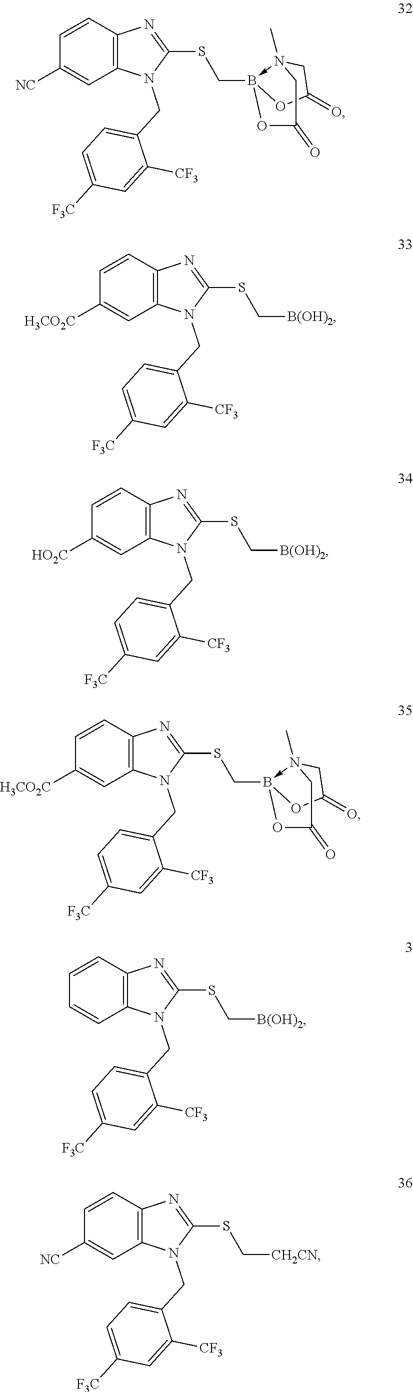 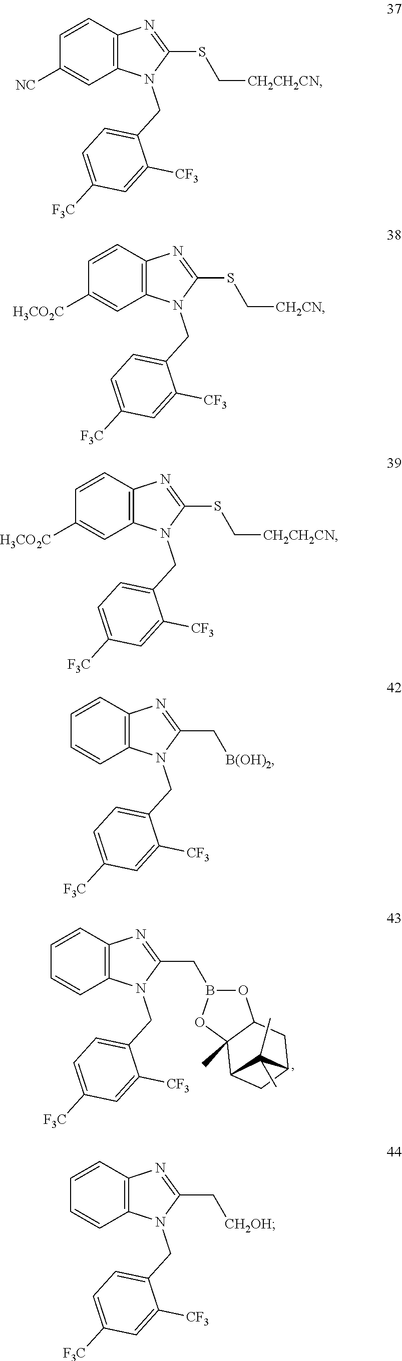
or a pharmaceutically acceptable salt thereof.

16. The compound of claim 1, wherein the compound is: 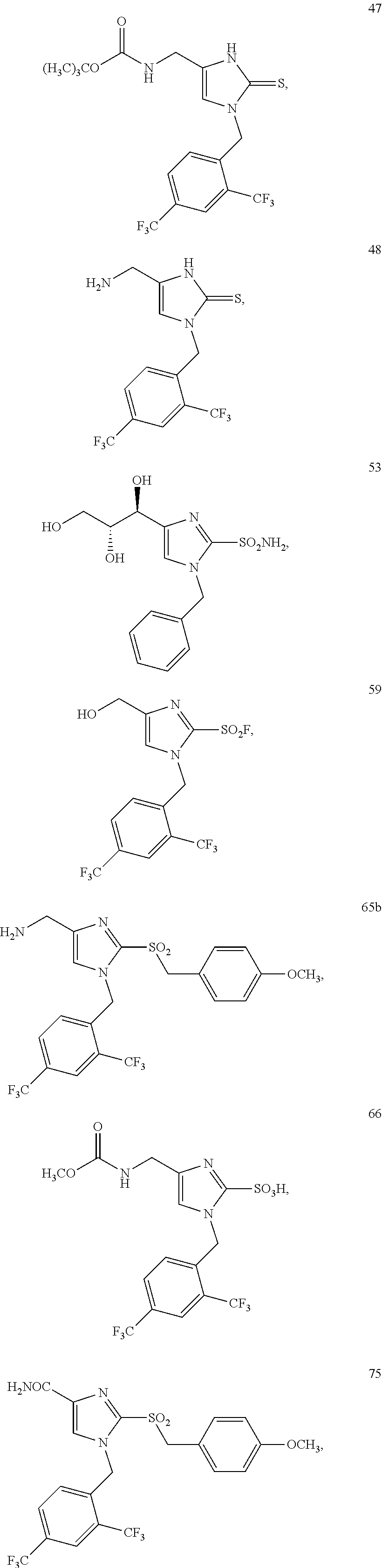
or a pharmaceutically acceptable salt thereof.

17. A pharmaceutical composition comprising a compound of claim 1 and a pharmaceutically acceptable diluent or carrier.

18. The pharmaceutical composition of claim 17, further comprising an antibiotic, wherein the antibiotic is optionally a β-lactam antibiotic.

19. A method for treating a bacterial infection, comprising administering a therapeutically effective amount of a compound of claim 1 to a subject infected with bacteria, wherein the bacterial infection is thereby treated.

20. (canceled)

21. A method for treating a methicillin-resistant Staphylococcus aureus (MRSA) infection, comprising administering a therapeutically effective amount of an antibiotic and a compound of claim 1 to a subject infected with MRSA, wherein the bacterial infection is thereby treated.

22-25. (canceled)
